(19) United States
(12) Patent Application Publication
(10) Pub. No.: US 2023/0174590 A1
(43) Pub. Date: Jun. 8, 2023

(54) SARS-COV-2 VIRAL PROTEINS AND USE THEREOF

(71) Applicants: Versitech Limited, Hong Kong (CN); Institut Pasteur, Paris (FR)

(72) Inventors: Asmaa Hachim, Hong Kong (CN); Niloufar Kavian, Hong Kong (CN); Leo Lit Man Poon, Hong Kong (CN); Malik Peiris, Hong Kong (CN); Sophie A. Valkenburg, Hong Kong (CN)

(21) Appl. No.: 17/997,434

(22) PCT Filed: Apr. 28, 2021
(86) PCT No.: PCT/CN2021/090684

§ 371 Date: Oct. 28, 2022

Related U.S. Application Data
(60) Provisional application No. 63/016,898, filed on Apr. 28, 2020.

(51) Int. Cl. C07K 14/005; G01N 33/569

(52) U.S. Cl. CPC C07K 14/005; G01N 33/56983; C07K 2319/60; C07K 2319/61; C12N 2770/20022; C12N 2770/20034; G01N 2333/165; G01N 2469/20

(57) ABSTRACT

Compositions and immunoassays for detecting SARS-CoV-2 antigen-specific antibodies in a biological sample are provided. The compositions are antigens from SARS-CoV-2 such as the proteins N, M, S, S1, S2′, NSP1, ORF3a, ORF3b, ORF7a, ORF7b and ORF8, fused to a light emitting protein. The compositions are useful in immunoassays for detecting antibodies to the SARS-CoV-2 antigens. A preferred immunoassay is the luciferase immunoprecipitation systems (LIPS) to detect SARS-CoV-2 antigen-specific antibodies with high sensitivity and specificity. The current or present exposure or infection with SARS-CoV-2 can be detected and/or diagnosed using the disclosed compositions and methods. Typically, the presence and/or elevated amount of SARS-CoV-2 antibodies in the individual’s biological sample compared to a control is indicative of current or past exposure or infection with SARS-CoV-2 as discussed herein.

### FIELD

The invention is generally in the field of immunogenic compositions of SARS-CoV-2 antigens and serological tests for COVID 19.

### BACKGROUND

The acute pandemic respiratory disease COVID-19 is caused by a novel coronavirus that belongs to the species Severe acute respiratory syndrome-related coronaviruses (SARS-CoV-2) (1, 2). Since December 2019, SARS-CoV-2 has become a pandemic virus resulting in 3 millions of deaths and 147 million cases of COVID-19 reported world-wide (as of 27^{th} of April 2021), along with deep socioeconomic implications.

The urgent need to control the pandemic by accelerating the development and mass production of efficacious vaccines against SARS-CoV-2 has led to unprecedented efforts in vaccinology with more than 300 vaccine candidates in various stages of development and human clinical trials (5). Though, the urgency in vaccine development and deployment to mitigate the pandemic has left some fundamental immunological research questions outstanding, especially regarding the range of virus immunogens that elicit antibody responses and protection, their kinetics, specificity, breadth, longevity and impact for long-term protection or immune-pathology. Yet, in recent months, efforts have been directed towards a better understanding of human humoral responses to SARS-CoV-2 infection. SARS-CoV-2 belongs to the β-coronavirus genus which also includes SARS-CoV emerged in 2002, and Middle Eastern Respiratory Syndrome Coronavirus (MERS-CoV) emerged in 2012, which are severe human diseases of zoonotic origin all three leading to acute respiratory syndrome (4). Ongoing research has reported the full genome sequence of all new variants (GISAID) (1), transcriptome (https://doi.org/10.1038/s41392-020-00237-0)(6) and immune response to infection (7). It has become critical to develop robust and reliable serological assays to characterize the abundance and duration of antibodies in virus-exposed individuals, along with differentiating current/past infection from vaccination. Serological testing is major for the diagnosis but also for the sero-epidemiology of SARS-CoV-2 virus infection and vaccines.

Several serology tests are currently in use which primarily assess Spike (S), and on occasion Nucleocapsid (N) antibodies (Yong, S.E.F. et al. Connecting clusters of COVID-19: an epidemiological and serological investigation. Lancet Infect Dis 20, 809-815 (2020)). Seroconversion to S and N generally occurs in the second or third week of illness and are not suitable for diagnosis of acute disease, and these assays have sub-optimal sensitivity and specificity. Moreover, most vaccines in development against COVID-19 target the Spike protein to elicit neutralizing antibodies to block infection, (Paules, C.I., Marston, H.D. & Fauci, A.S. Coronavirus Infections-More Than Just the Common Cold. JAMA 323, 707-708 (2020)) as the S protein contains the receptor binding domain (RBD) which is critical for viral entry (Yuan, M. et al. A highly conserved cryptic epitope in the receptor-binding domains of SARS-CoV-2 and SARS-CoV. Science 368, 630-633 (2020). The presence of anti-S antibodies in individuals could therefore be the result of both infection and vaccination. However, it is unclear whether neutralizing antibodies to S protein are the major contributor to a protective immune response (Wu, F. et al. Neutralizing antibody responses to SARS-CoV-2 in a COVID-19 recovered patient cohort and their implications medRxiv (2020)). Neutralizing antibodies develop in most COVID-19 patients, however, it is reported by some studies that a proportion of RT-PCR confirmed COVID-19 patients do not develop robust antibody responses measured by neutralization tests.

Also, many cases of reinfection in previously infected and vaccinated people, termed “breakthrough infection”, have been reported over the last months, with 5,700 cases already identified in the USA alone (https://www.cdc. gov/vaccines/covid-19/health-departments/breakthrough-cases. html), raising the issue of low antibody responses from the primary infection (Paul K.S. Chan, Serologic Responses in Healthy Adult with SARS-CoV-2 Reinfection, Hong Kong, August 2020. Emerg Infect Dis. 2020 Dec).

The possibility of low or no antibody responses detection by traditional serological approaches may lead to an underestimation of past asymptomatic and mild infection.

Therefore, a broader landscape of antibody responses to a range of viral proteins needs to be assessed to better detect the immunogenicity of SARS-CoV-2 infection, discriminate past-infection from vaccination, and understand pathogenesis and immunity.

### SUMMARY

Compositions and immunoassays for detecting SARS-CoV-2 antigen-specific antibodies in a biological sample are provided. The compositions are immunogenic and include an antigen from SARS-CoV-2. In a preferred embodiment the immunogenic compositions include SARS-CoV-2 antigens ORF8, ORF3b and/or N proteins or fragments thereof, as antigens. In some forms, the immunogenic compositions included are fusion proteins including SARS-CoV-2 antigens proteins N, M, S, S1, S2′, NSP1, ORF3a, ORF3b, ORF7a, ORF7b and ORF8, fused to a light emitting protein. In some forms, the immunogenic compositions included are fusion proteins including non-structural proteins ORF8, ORF3b and/or the structural protein N, which are fused to a light emitting protein. In some forms, the immunogenic compositions included are fusion proteins including non-structural proteins ORF8 and/or ORF3b, which are fused to a light emitting protein. The disclosed compositions also include expression vectors which include nucleic acid sequences encoding SARS-CoV-2 N, M, S, S1, S2′, NSP1, ORF3a, ORF3b, ORF7a, ORF7b and ORF8 to be expressed as a fusion with a light emitting protein.

In one preferred embodiment, the immunoassays utilize the luciferase immunoprecipitation systems (LIPS) to detect SARS-CoV-2 antigen -specific antibodies with high sensitivity and specificity. The method includes providing a fusion protein comprising an antigen fused to a light-emitting protein; contacting the biological fluid sample with the fusion protein. If antigen specific antibodies are present in the biological fluid sample, a complex formation of the SARS-CoV-2 antigen fused to light emitting protein with the specific antibody is formed and pulled down using beads. The level of antibodies can then be detected by substrate addition which is proportional to the number of antibodies bound. The immunoglobulin-binding protein can be Protein A, Protein G, Protein A/G, Protein L or a secondary immunoglobulin molecule. In a preferred embodiment, the immunoglobulin-binding protein is the protein A/G. The light-emitting protein is preferably a luciferase, such as a Renilla luciferase. The LIPS assay in some embodiments, includes ORF8 as the only SARS-CoV-2 antigen, and a subject is identified as COVID-19 positive on the basis of a LIPS and ELISA assay including ORF8 as the only SARS-CoV-2 antigen. In some other embodiments, SARS-CoV-2 antigens ORF8, ORF3b and/or N. In some embodiments, detecting SARS-CoV-2 antigen-specific antibodies is at early timepoints, prior to day 14 since exposure/infection. In some embodiments, the biological fluid sample is from children.

In other embodiments, the immunoassays include, but are not limited to, radioimmunoassays, ELISAs, immunoprecipitation assays, Western blot, and fluorescent immunoassays. The disclosed immunogenic compositions can be used to raise antibodies against ORF8, ORF3b, which can be applied in immunoassays such as ELISAs and dipstick assays. In these embodiments, the immunoassay preferably detects SARS-CoV-2 antigens ORF8, ORF3b and/or N proteins.

For example, in preferred embodiments, the ELISA is an “indirect ELISA.” Indirect ELISAs can include one or more of the following steps. A SARS-CoV-2 protein(s), or fragment(s) or fusion(s) thereof (i.e., the antigen) is added, linked, or otherwise bound to a surface. The biological sample (which may contain SARS-CoV-2 antibodies) can be added and any antibodies present allowed to specifically bind to the antigen. A secondary antibody that can detect the presence of the SARS-CoV-2 antibodies (e.g., an antibody specific for a constant region of antibodies produced by subject from which the sample was obtain (i.e., human Ig constant region) with an attached (conjugated) enzyme or other detectable label is added. If required for detection, a substrate for the detectable label (e.g., enzyme) is then added. The assays can include incubation and washing steps.

In some embodiments, the assay is in a dip stick format. The dipstick can contain SARS-CoV-2 antigen. The dipstick is contacted with biological sample to allow antibodies present in the sample, e.g. from saliva or blood (as a dry rapid blot test) as the biological sample, to bind to the antigen, and can be processed similar to an ELISA. In the dipstick format, the assay can be processed using cuvettes. For example, to detect immunoglobulin G (IgG) the dipstick can be processed through reaction cuvettes containing biological sample, enzyme-conjugated secondary (e.g., anti-human IgG and IgM antibody, etc.), and optionally substrate, etc.

The current or present exposure or infection with SARS-CoV-2 can be detected and/or diagnosed and/or treated using the disclosed compositions and methods. Typically the presence and/or elevated amount of SARS-CoV-2 antibodies the individual’s biological sample compared to a control is indicative of past, current or present exposure or infection with SARS-CoV-2 as discussed herein. For example, the method for assisting in the detection or diagnosis of current or present exposure or infection with SARS-CoV-2 in a subject can include determining the presence or level of antibodies against one or more SARS-CoV-2 proteins, fragments, or fusion proteins in a biological sample from the subject, wherein the presence of, or an elevated level of, the antibodies in the biological sample relative to the level of antibodies in a control is indicative of past, current or present exposure or infection with SARS-CoV-2.

In some embodiments, the method is combined with a method of detecting SARS-CoV-2. If virus is detected, the subject may have a current exposure or infection. If virus is not detected, the subject may not have a current exposure or infection, and the presence of antibodies may be due to a previous exposure or infection.

Any of the methods can be combined with a method of treatment. In preferred embodiments, the method of treatment includes administering the subject an effective amount of an anti-viral therapy, analgesic therapy, fever reducers, cough suppressants, and/or respiratory assistance (e.g., ventilator treatment).

Assays and kits that include reagents for the detection and qualitative or quantitative measurement of SARS-CoV-2 antibodies in a subject’s biological sample are also provided. For example, if detection of the SARS-CoV-2 antibodies is by means of ELISA, components of the assay or kit can include SARS-CoV-2 protein(s), or fragment(s) or fusion(s) thereof, optional immobilized on a surface such as beads or assay dish or wells thereof, and optionally a detection means such as an antibody that can bind to the SARS-CoV-2 antibody (e.g., an antibody specific for a constant region of antibodies produced by subject from which the sample was obtain (i.e., human IgG constant region) optionally, with an attached (conjugated) enzyme or other detectable label e.g., fluorescent dye or radioactive label.

### DESCRIPTION OF THE FIGURES

FIGS. 1A-1E show detection of SARS-CoV-2 structural proteins antibodies by LIPS. FIG. 1A illustrates the SARS-CoV-2 genome ORF organisation showing the AAAAA at the 3′ end (not to scale). FIGS. 1B-1E are bar graphs showing the results of antibodies against the four SARS-CoV-2 structural proteins Spike (S), Nucleocapsid (N), Membrane (M), Envelope (E) measured by LIPS from COVID-19 patients, and age matched healthy negative controls. Data represents the mean +/- stdev, and individual responses (n=15). Background values were subtracted. Experiments were repeated twice. P values were calculated using the Mann-Whitney U test. * shows statistical significance between COVID-19 patients versus healthy controls. ns p=0.0591 *p<0.05, **p<0.01, ***p<0.005. Experiments were repeated twice.

FIGS. 2A-2C show LIPS detection of antibody levels to the SARS-CoV-2 S protein subunits. FIG. 2A. Antibodies against the S subunits S1, S2, and S2′ by LIPS from COVID-19 patients and healthy controls. FIG. 2B. Full S, S1, S2, S2′ Abs LIPS titers in COVID-19 patients with low microneutralization (MN) titers (<160) versus high MN titers (≥160). FIG. 2C. Full S Abs LIPS titers in COVID-19 patients with low ELISA S IgG titers (<1) versus high ELISA IgG titers (≥1). Data represents the mean +/- stdev, and individual responses (n=15). Background values were subtracted. Experiments were repeated twice. P values were calculated using the Mann-Whitney U test. * shows statistical significance between COVID -19 patients versus healthy negative controls. *p<0.05, **p<0.01, ***p<0.005.

FIGS. 3A-3J show detection of SARS-CoV-2 non-structural proteins antibodies by LIPS. FIGS. 3A-3H. Antibodies against NSP1 (in ORF1ab), and other ORFs (ORF3a, ORF 3b, ORF 6, ORF 7a, ORF 7b, ORF 8 and ORF 10) were measured by LIPS to cover all the ORFs of the virus. Data represents the mean +/- stdev, and individual responses (n=15). FIG. 3I. shows sensitivity and specificity performances of the 11 LIPS tests. FIG. 3J. differences of the antibody titer means between COVID-19 and negative control populations.

FIGS. 4A-4I show combining LIPS tests as a diagnostic tool for COVID-19. Cumulative antibody LIPS levels to the SARS-CoV-2 antigens in COVID-19 patients and healthy controls, for (FIG. 4A) 11 relevant antigens (N, M, S, S1, S2′, NSP1, ORF3a, ORF3b, ORF7a, ORF7b, ORF10) (FIG. 4B) three most sensitive antigens (N, ORF3b, and ORF8), (FIG. 4C) selective antigens (ORF3b, and ORF8), and (FIG. 4D) 3 S relevant antigens (S, S1, S2′). (FIGS. 4E-4F) Detection of early time-points patients only with the Sum of (FIG. 4E) 11 relevant Antigens and (FIG. 4F) three most sensitive antigens (N+ORF3b+ORF8). Data represents the mean +/- stdev, and individual responses (n=15). The cut-off value of the sums is shown by the dotted line and was based on the mean + three stdev of the negative control group. Blue dots represents COVID-19 patients data points prior to day 14. (FIGS. 4H-4I) as (FIG. 4H) scatter plot including the 11 relevant antigens, and as (FIG. 4I) pie chart excluding N. Each symbol represents the mean +/- stdev of the differences between COVID-19 and healthy negative groups, for the 11 relevant antibody levels in light units (LU). **** p<0.0001 versus all other antigens, otherwise indicated.

FIGS. 5A-5D show S and N IgG responses by ELISA and LIPS. FIG. 5A shows full S IgG ELISA for COVID-19 patients and healthy negative controls. FIG. 5B shows Pearson Spearman correlation of full S ELISA and LIPS of COVID-19 patients. FIG. 5C shows N IgG ELISA for COVID-19 patients and healthy controls. Data represents the mean +/- stdev, and individual responses (n=15). Experiments were repeated twice. P values were calculated using the Mann-Whitney U test. * shows statistical significance between COVID-19 patients versus healthy controls. *p<0.05, **p<0.01, ***p<0.005. FIG. 5D shows Pearson Spearman correlation of N ELISA and LIPS of COVID-19 patients. FIG. 5E Pre- and post-adsorption LIPS antibody results of COVID-19 patients (n=3). Experiments were repeated twice.

FIGS. 6A and 6B show absence of sex- and age-related effect on the production of SARS-CoV-2 antibodies by LIPS. Antibodies against SARS-CoV-2 antigens were measured by LIPS in COVID-19 patients and stratified by gender (FIG. 6A) and age (<60, and >60 years of age) (FIG. 6B). Data represents the mean +/stdev, and individual COVID-19 responses (n=15). Experiments were repeated twice. P values were calculated using the Mann-Whitney U test. * shows statistical significance between COVID-19 patients versus healthy controls. *p<0.05, **p<0.01, ***p<0.005.

FIGS. 7A-G. Combining LIPS tests as a diagnostic tool for COVID-19. (7F) Cluster of points representing each COVID-19 patient in red and negative control in grey in the plane (ORF3b, ORF8). The equation of the line 0.2187x + 0.5927y=4643.19. (7G) Sensitivity and specificity performances of the N, ORF3b, ORF8 and their sums (from 7A-E), and cluster analysis (from 7F). The contour red line shows the highest sensitivity and specificity. Two-sided P values were calculated using the Mann-Whitney U test. * shows statistical significance between COVID-19 patients versus negative controls. *p<0.05, **p<0.01, ***p<0.005.

FIGS. 8A-8F. SARS-CoV-2 antibody responses over time. (8A) Sensitivity performances of the N, ORF3b, ORF8 and their sums for early time-points (prior to day 14). The contour red line shows the highest sensitivity and specificity. Detection of early time-points patients only (≤ day 14) with the sum of three most sensitive antigens (N+ORF3b+ORF8) (8B) and ORF3b+ORF8 (8C). (8D) Longitudinal time-point patients (n=14) responses for N (red), ORF3b (pink), ORF8 (green) and Spike (blue). The x axis represents time-point (to scale) after onset of symptoms in days for each patient. On the y axis each unit represents 10³ for ORF3b, ORF8 and Spike, and 10⁶ for N (origin shifted for the sake of clarity). (8E) Linear regression of full S LU versus ORF8 LU of COVID-19 patients (from 8D) (R²=0.66902, p<0.0001, two-sided P value). (8F) Fold changes from earliest available acute time point sample (from 8D) to convalescence (14-30 days post symptom onset) and long term memory (>31 days post symptom onset) of S, N, ORF8 and ORF3b LU. * shows statistical significance by one sample t test versus a hypothetical value of 1-fold change *p<0.05

FIG. 9. Detection of early time-points patients only (≤ day 14) for N, ORF3b and ORF8. LIPS results from with N (9A), ORF3b (9B), ORF8 LIPS tests (9C) and N+ORF8 (9D). Data represents individual responses (n=51). The cut-off value of the sums is shown by the dotted line and was based on the mean plus three std-dev of the negative control group. Experiments were repeated twice.

FIG. 10. α-and β-HCoV positive samples do not cross-react with N, ORF3b and ORF8 SARS-CoV-2 LIPS assays. OC43 , 229E, and NL63 Spike ELISAs were performed on n=176 COVID-19 negative control cohort samples (Negative pre-pandemic controls used in this study). Graphs (10a-c) represent the LIPS results for SARS-CoV-2 N, ORF3b and ORF8 according to the positivity/negativity for OC43 Spike by ELISA in the COVID-19 negative sample cohort. Graphs (10d-f) represent the LIPS results for N, ORF3b and ORF8 according positivity/negativity for 229E Spike by ELISA. Graphs (10g-i) represent the LIPS results for SARS-CoV-2 N, ORF3b and ORF8 according to the positivity/negativity for NL63 Spike by ELISA in the COVID-19 negative sample cohort. The dotted-line represents the cut-offs used in the FIG. 7. (10j) Percentages of OC43, 229E, and NL63 Spike IgG in the negative cohort. ELISAs were performed on n=176 COVID-19 negative control cohort samples. Positivity in HCoV ELISAs was defined as O.D. >1 and negativity as O.D. <1. Two-sided P values were calculated using the Mann-Whitney U test. Ns means no significant difference.

FIG. 11. Comparison of antibody responses to SARS-CoV-2 structural proteins in children and in adults with COVID-19. Antibodies against the SARS-CoV-2 structural proteins Spike S1 subunit (S1) (a), Spike S2 subunit (b), Spike S2′ subunit (c), Nucleocapsid (N) (d), Membrane (M), and Envelope (E) (f) were measured by LIPS from samples from pediatrics COVID-19 (n=254) or adult patients (n=36), and negative controls (n=33) (cohort described in Table 6). Background no plasma values were subtracted. Experiments were repeated twice. All data represents individual responses, and the mean +/- stdev. Two-sided P values were calculated using the Mann-Whitney U test. * shows statistical significance between COVID-19 patients versus negative controls. **p<0.01, ***p<0.001, **** p<0.0001.

FIG. 12. Antibody responses to SARS-CoV-2 non-structural proteins and ORFs are lower in magnitude in children than in adults with COVID-19 but represent globally a higher proportion of the SARS-CoV-2 humoral response. Antibodies against NSP1 (a) (in ORF1ab), and other ORFs (ORF3a (b), ORF3b (c), ORF6 (d), ORF7a (e), ORF7b (f), ORF8 (g) and ORF10 (h)) were measured in pediatric (n=254) and adult (n=36) COVID-19 cases and negative controls (n=33) by LIPS to cover all the ORFs of the virus. (i) A heatmap comparing the mean titres (LU) for structural (N, S, S1, S2′, S2, M, E) and accessory proteins (NSP1, ORF3a, ORF3b, ORF6, ORF7a, ORF7b, ORF10) responses in the COVID-19 pediatric and adult populations and Negatives. (j) Percentages of single antibody levels to SARS-CoV-2 antigens of the cumulative SARS-COV-2 antibody response in COVID-19 children and adults for the 14 antigens. Experiments were repeated twice. Two-sided P values were calculated using the Mann-Whitney U test. * shows statistical significance between COVID-19 patients versus negative controls. *p<0.05, **p<0.01, ***p<0.005, **** p<0.0001. Data in (a-h) represents the individual responses and mean +/stdev, data in (i) represents mean values (LU), data in (j) represents percentages.

FIG. 13. Representation of the pediatric COVID-19 population as a cluster of points for relevant antibody combinations and Principal Component Analysis (PCA). (a-b). Cluster representation of S1, S2′, S2 antibodies combination. (a) shows the pediatric COVID-19 population versus the adult COVID-19 population, (b) shows the pediatric COVID-19 population versus the negative population. (c) Cluster representation of N, ORF3b, ORF8 antibodies combination, for the pediatric COVID-19 population versus the adult COVID-19 population and the negative population. Patients are presented according to their values of SARS-CoV-2 individual LIPS antibodies as (x, y, z) in the space. Pediatric COVID-19 patients (n=144) are represented as red dots. COVID-19 adult patients (n=36 in (a-b) and n=24 in (c)) are represented in blue. The negative population (n=28) is represented in gray. (d-f) PCA of 14 antibodies analyzed in COVID-19 pediatric patients. Dim1 explains 21% of the variation, while Dim2 explains 15% of the variation. (d) Correlation circle and contributions. The scale of contributions is indicated on the right). (e) Contribution of variables on dimensions 1 and 2. The red dashed line on the graph above indicates the expected average contribution. (f) Factorial plot of PCA on dimension 1 and 2. The plot is colored by sample types, the largest point in shape in each group is the group mean point (circle is for Adult positives, triangle for Negatives and squares for Pediatric positives).

FIG. 14. Asymptomatic and mildly symptomatic children do not display different antibody landscapes. Pediatric and adult samples were stratified according to the symptom score of the patients (asymptomatic «asympto» (pediatric COVID-19 n=98, adults COVID-19 n=9) versus symptomatic «sympto» (pediatric COVID-19 n=156, adults COVID n=27)), data from FIGS. 1 and 2 were analyzed according to “asympto” and “sympto”. (a) Antibodies against the SARS-CoV-2 structural proteins S1, S2, S2′, N, E, and M by LIPS. (b) Antibodies against SARS-COV-2 NSP1 (in ORF1ab), and all other ORFs (ORF3a, ORF3b, ORF6, ORF7a, ORF7b, ORF8 and ORF10). Two-sided P values were calculated using the Mann-Whitney U test. * shows statistical significance between COVID-19 patients versus negative controls. *p<0.05, **p<0.01, * * *p<0.005, **** p<0.0001. All data represent individual responses and the mean +/- stdev.

FIG. 15. A unique antibody landscape is specific of early time-point samples (< day 14). Pediatric samples were stratified according to the time-point of collection, and data from FIGS. 1 and 2 were analyzed according to acute (<day 14, n=119) and later time-points (≥day 14, n=135). (a) Antibodies against the SARS-CoV-2 structural proteins S1, S2, S2′, N, E, and M by LIPS. (b) Antibodies against NSP1 (in ORF1ab), and all other ORFs (ORF3a, ORF3b, ORF6, ORF7a, ORF7b, ORF8 and ORF10). P values were calculated using the student t test. * shows statistical significance between acute time-point pediatric COVID-19 patients versus late time-point pediatric COVID-19 patients. *p<0.05, **p<0.01, * * *p<0.005, **** p<0.0001. All data represent individual responses and the mean +/- stdev.

FIG. 16. Longitudinal stability of antibody responses for structural and non-structural SARS-CoV-2 proteins in COVID-19 children. (a) Number of longitudinal patients with either 2, 3 or 4 blood draws from 58 pediatric COVID-19 cases. (b) Sample collection time-line (days post infection). (c) A linear trend on log₁₀ LIPS values was fitted for longitudinal samples for S1, S2′, N, M E, NSP1, ORF3a, ORF3b, ORF7a, ORF7b, ORF8 (n=58 pediatric COVID-19 patients).

FIG. 17. IFN-α producing pediatric patients display a different landscape of antibody response to SARS-CoV-2 accessory proteins. (a) Cluster representation of antibodies to the accessory proteins ORF3b, ORF7a, ORF6 (x, y, z) for the COVID-19 pediatric samples (red, n= 144) versus the COVID-19 adult samples (blue, n= 27). (b) Plasma IFN-α concentrations (pg/ml) in pediatric (n=48) and adult COVID-19 cases (n=18) early timepoint samples (< day 7). Data represents individual responses and the mean +/- stdev. (c) Pie charts of the cumulative antibody responses to the relevant SARS-CoV-2 structural and non-structural protein antigens (excluding N) in COVID-19 pediatric cohort stratified (positive/negative) by their IFN-α responses. (d) Individual data for IFN-α⁺ pediatric cases viral loads by RT-PCR, ORF3b, ORF6, and ORF7b LIPS LU. P values were calculated using Chi-squared test between the mean of IFNα-pediatric COVID-19 patients (N=45) and the IFNα⁺ (N=3). ns, p=0.0591 *p<0.05, **p<0.01, ***p<0.001, **** p<0.0001.

FIG. 18. Examples of commercially available SARS-CoV-2 antibody detection kits rely on S and/or N and their sensitivity and specificity.

FIG. 19. ORF8 and ORF3b are more stable than S/N, mutations in those regions are mostly synonymous, aside from variants of concern B1.1.7 and B1.351.

FIG. 20. Common Cold Coronaviruses share high homology with S and N (which can lead to cross-reactivity), whilst ORF8 and ORF3b are specific to SARS-CoV-2. (A-D) Rate of spike mutations in SARS-CoV-2 across different clusters and domains. (E) % amino acid sequence homology of SARS-CoV-2 proteins versus other coronaviruses.

FIG. 21. ORF8 and ORF3b showed higher sensitivity in diagnosing re-infection by SARS-CoV-2 variant. (A) Schematic of 64E stop mutation in ORF8 of 2^{nd} infection and 1st infection. (B-D) Antibodies against S, N, ORF8+3b and ORF8 from d10, d43 and d5 of secondary infection in serum were measured by LIPS. The dotted line represents the cut-off calculated by the mean of negatives plus three standard deviations.

FIG. 22. ORF3b and ORF8 are stable overtime in adults (day 0- day 100) and children (day 0- day 204) making them effective serological markers at all time-points of infection. (a) Number of longitudinal patients with either 2, 3 or 4 blood draws from 58 pediatric COVID-19 cases. (b) Sample collection time-line (days post infection). (c) A linear trend on log₁₀ LIPS values was fitted for longitudinal samples for ORF3b and ORF8 (n=58 pediatric COVID-19 patients). (d) Fold changes from earliest available acute time point sample to convalescence (14-30 days post symptom onset) and long-term memory (>31 days post symptom onset) of S, N, ORF8 and ORF3b LU in adult COVID-19 plasma. * shows statistical significance by one sample t test versus a hypothetical value of 1-fold change *p<0.05

FIG. 23. ORF8 and ORF3b are sensitive and specific markers of SARS-CoV-2 infection (a-d)Antibodies against the three relevant SARS-CoV-2 Nucleocapsid (N) (a), ORF3b (b), ORF8 (c), were measured by LIPS from COVID-19 samples (n=84) and negative controls (n=176). Sum of antibody LIPS levels to N+ORF3b+ORF8 (d) and ORF3b+ORF8 (e) SARS-CoV-2 antigens in COVID-19 patients and negative controls. Data represents the mean +/- stdev, and individual responses. Background values were subtracted. Experiments were repeated twice. (f) Sensitivity and specificity performances of the N, ORF3b, ORF8 and their sums and cluster analysis. The contour red line shows the highest sensitivity and specificity. (g) Antibodies against ORF8 protein (from Masashi Mori, Ishiwaka University, Japan) in plasma of COVID-19 patients (n=550) and negatives (n=184) in ELISA. The dotted line represents the cut-off calculated as the mean of negatives plus three standard deviations.(h) Correlation of anti-ORF8 antibodies in COVID-19 patients with time in days (R²=0.05944, ns)

FIG. 24. ORF8 ELISA is an accurate diagnostic tools for COVID-19 children and at early all time-points of infection. (a-c)Antibodies against ORF8 in plasma of COVID-19 children (n=243) and negatives (n=184). The data is further stratified (b) from to asymptomatic (n=63) and symptomatic patients (n=180) and (c) to early (n=115) and late (n=128). (d-f) Antibodies against ORF8 in plasma of COVID-19 adults (n=304). The data is further stratified (e) from to asymptomatic (n=40) and symptomatic patients (n=227) and (c) to early (n=178)and late (n=90). The dotted line represents the cut-off calculated as the mean of negatives plus three standard deviations.

FIG. 25. In house ELISA of Spike and Nucleocapsid using SinoBiological proteins for comparison as a diagnostic. (a-b) Antibodies against full Spike and Nucleocapsid (from Sinobiological) in COVID-19 adults (n=84) and negatives (n=100). The dotted line represents the cut-off calculated as the mean of negatives plus three standard deviations.

FIG. 26. ORF8 is detected in infected SARS-CoV-2 cells and co-localises with M and ERGIC-53 highlighting its potential as an antigen test. (a) Linear regression of full S LU versus ORF8 LU of COVID-19 patients (from 8D) (R2=0.66902, p<0.0001, two-sided P value). (b) Immunofluorescence staining of ORF8 and structural protein M of SARS-CoV-2 reveals a colocalization of the proteins (MOI2, 24 h p.i. 40x). (c) Immunofluorescence staining of ORF8 and structural protein Spike and ERGIC-53 (MOI2, 24 h p.i. 40x) reveals co-localisation with ERGIC-53 and abundance of ORF8 protein in infected cells. Experiments were repeated three times.

### 5. DETAILED DESCRIPTION

SARS-CoV-2 antibody testing is an important component of the options for diagnosis of recent and past COVID-19 infection. Antibody tests are important for determining infection attack rates in the population, population immunity and informing vaccine development. Reported here, for the first time, is the detection of antibody responses directed against an extensive spectrum of the SARS-CoV-2 antigens. Several approaches have been developed to measure SARS-CoV-2-specific antibodies, including micro neutralization assays (virus or pseudo virus-based (25)), ELISA assays (11, 26), immunofluorescence (7), colloidal gold-based immunochromatographic assays (27), and peptide/protein microarray (28, 29). Most commercially available or published serological tests use only the S antigen, with a few using both the S and N antigens (11, 36, 37). Using extensive testing of the virus antigens, the present studies showed that additional targets are important for early detection of antibody responses and identification of COVID-19 patients. Therefore, the approach of combining several relevant antigens that are unique to SARS-CoV-2, and also immunogenic boosting specific antibodies would overcome issues of cross-reactivity and increase the sensitivity of serological assays.

### I. Definitions

As used herein, a “vector” is a replicon, such as a plasmid, phage, or cosmid, into which another DNA segment may be inserted so as to bring about the replication of the inserted segment. The vectors described herein can be expression vectors. As used herein, an “expression vector” is a vector that includes one or more expression control sequences.

As used herein, an “expression control sequence” is a DNA sequence that controls and regulates the transcription and/or translation of another DNA sequence.

“Operably linked” refers to a juxtaposition wherein the components are configured so as to perform their usual function. For example, control sequences or promoters operably linked to a coding sequence are capable of effecting the expression of the coding sequence, and an organelle localization sequence operably linked to protein will direct the linked protein to be localized at the specific organelle.

As used herein, the term “host cell” refers to a cell into which a recombinant vector can be introduced.

As used herein, “transformed” and “transfected” encompass the introduction of a nucleic acid (e.g. a vector) into a cell by a number of techniques known in the art. As used herein, a “label” or a “detectable moiety” is a composition detectable by spectroscopic, photochemical, biochemical, radiographic, immunochemical, chemical, or other physical means. For example, useful labels include ³²P, fluorescent dyes, electron-dense reagents, enzymes (e.g., as commonly used in an ELISA), biotin, digoxigenin, or haptens and proteins or other entities which can be made detectable, e.g., by incorporating a radiolabel into the peptide or used to detect antibodies specifically reactive with the peptide. The labels may be incorporated into nucleic acids, proteins and antibodies at any position. Any method known in the art for conjugating the antibody to the label may be employed, e.g., using methods described in Hermanson, Bioconjugate Techniques 1996, Academic Press, Inc., San Diego.

### II. Compositions

The disclosed compositions include SARS-CoV-2 antigens that can be used alone, or in combination in a diagnostic for COVID 19 and/or detect the presence of SARS-CoV-2-specific antibodies in a subject.

SARS-CoV-2 has 12 putative functional open reading frames (ORF) and shares 82% nucleotide homology with SARS-CoV (8). There are at least four structural proteins in SARS-CoV-2: Spike (S), Envelope (E), Membrane (M), Nucleocapsid (N). The trimer S protein is cleaved into S1, containing the receptor binding domain, and S2 subunits (8, 9), and S2 is further cleaved into S2′ to form the viral fusion peptide (6). The S1 subunit of SARS-CoV-2 shares about 70% identity with the SARS-CoV, whereas the identity of the S2 subunit is up to 99% with some evidence of cross-reactivity between the viruses (11). Apart from these structural proteins, the SARS-CoV-2 genome encodes for around 20 putative non-structural proteins (8). ORF1a/b encodes for a large polyprotein that is proteolytically cleaved into 16 non-structural proteins (NSP1-16). Extra ORFs, such as ORF3a, 3b, 6, 7a, 7b, 8 and 10 may encode for proteins but their functions are unknown.

### A. Fusion Proteins

The disclosed compositions include fusion proteins of the structural proteins N, M, S, S1, S2′, the large NSP1, or the amino acids encoded by ORF3a, ORF3b, ORF7a, ORF7b and ORF8 SARS-CoV-2, with light-emitting protein. The light-emitting protein comprises a fluorescent protein or a luciferase, such as a Renilla luciferase, a Gaussia luciferase, a modified (optimized) Oplophorus gracilirostris luciferase (for example, NANOLUC™ (is commercially available), firefly luciferase and bacterial luciferase), a firefly luciferase or a bacterial luciferase. In a preferred embodiment, light-emitting protein is a Renilla luciferase.

### B. Vectors and Cells Including SARS-CoV-2 Antigen Fusion Protein Encoding Domain

Vectors including nucleic acids encoding a fusion of SARS-CoV-2 antigen and light emitting proteins as well are cells carrying these vectors also provided. Nucleic acids, encoding the structural proteins N (for example by SEQ ID NO:7), M (for example by SEQ ID NO:5), S (for example by SEQ ID NO:8), S1 (for example by SEQ ID NO:9), S2′(for example by SEQ ID NO: 11), NSP1 (for example by SEQ ID NO:12), ORF3a (for example, SEQ ID NO:2), ORF3b (for example, SEQ ID NO:3), ORF7a (for example, SEQ ID NO:14), ORF7b (for example, SEQ ID NO:15) and ORF8 (for example, SEQ ID NO:6) of SARS-CoV-2, can be inserted into vectors for expression in cells. Preferred cells include in Cos1 or other cells (e.g., HEK 293). The vectors include any of SEQ ID NOS: 2, 3, 5-8, 9, 11-12 and 14-15, operably linked to a promoter. A preferred vector is a plasmid such as pREN2. Antigen fusions to the C-terminal of Ruc are made by cloning into the pREN2 vector. While the pREN3S vector generates N-terminal fusions.

Nucleic acids in vectors can be operably linked to one or more expression control sequences. For example, the control sequence can be incorporated into a genetic construct so that expression control sequences effectively control expression of a coding sequence of interest. Examples of expression control sequences include promoters, enhancers, and transcription terminating regions. A promoter is an expression control sequence composed of a region of a DNA molecule, typically within 100 nucleotides upstream of the point at which transcription starts (generally near the initiation site for RNA polymerase II). To bring a coding sequence under the control of a promoter, it is necessary to position the translation initiation site of the translational reading frame of the polypeptide between one and about fifty nucleotides downstream of the promoter. Enhancers provide expression specificity in terms of time, location, and level. Unlike promoters, enhancers can function when located at various distances from the transcription site. An enhancer also can be located downstream from the transcription initiation site. A coding sequence is “operably linked” and “under the control” of expression control sequences in a cell when RNA polymerase is able to transcribe the coding sequence into mRNA, which then can be translated into the protein encoded by the coding sequence.

Suitable expression vectors include, without limitation, plasmids and viral vectors derived from, for example, bacteriophage, baculoviruses, tobacco mosaic virus, herpes viruses, cytomegalo virus, retroviruses, vaccinia viruses, adenoviruses, and adeno-associated viruses. Numerous vectors and expression systems are commercially available from such corporations as Novagen (Madison, WI), Clontech (Palo Alto, CA), Stratagene (La Jolla, CA), and Invitrogen Life Technologies (Carlsbad, CA). Recent transfection studies have investigated minicircle DNA (mcDNA), nucleic acids that are derived from pDNA by recombination that removes bacterial sequences. L1 RNA can be introduced into host cells using mcDNA using methods known in the art (Mun et al. Biomaterials, 2016; 101: 310-320).

The vectors and cell including the vectors are used in a Luciferase immunoprecipitation system.

In general, the Luciferase immunoprecipitation systems harness light-emitting recombinant antigen-fusion proteins to quantitatively measure patient antibody titers. Renilla luciferase (Ruc), derived from the sea pansy, is preferably used for generating antigen fusions. Ruc is an ideal reporter owing to its small size (i.e., molecular weight: 30 kDa, approximately the same size as green fluorescent protein), wide linear detection range (100-107 light units [LUs]) and a complete lack of antigenicity with human and other animal sera. Other luciferases, including the 60-kDa firefly luciferase, can be used. Once light emitting protein-antigen mammalian expression plasmids are generated, these constructs are transiently transfected into a suitable mammalian cell such as Cos1 or other cells (e.g., HEK 293) to produce the Ruc antigens. After 48 h of transfection, the cells are scraped in cold lysis buffer containing glycerol, cleared by centrifugation and used directly in the LIPS assay.

### III. Methods of Using

The disclosed SARS-CoV-2 proteins, fragments, and fusions can be used in assays designed to identify the presence of antibodies against one or more SARS-CoV-2 proteins, fragments, or fusion proteins (“anti-SARS-CoV-2 antibodies”, “SARS-CoV-2 antibodies”, etc.) in a biological sample obtained from a subject to determine if the subject has been exposed to, or infected with, SARS-CoV-2.

A biological sample that may contain SARS-CoV-2 antibodies can be obtained from an individual. If the biological sample is of tissue or cellular origin, the sample is solubilized in a lysis buffer optionally containing a chaotropic agent, detergent, reducing agent, buffer, and salts. The sample is preferably a biological fluid sample taken from a subject. Examples of biological samples include urine, barbotage, blood, serum, plasma, tears, saliva, cerebrospinal fluid, tissue, lymph, synovial fluid, or sputum etc. In a preferred embodiment, the biological fluid is whole blood, or more preferably serum or plasma. Serum is the component of whole blood that is neither a blood cell (serum does not contain white or red blood cells) nor a clotting factor. It is the blood plasma with the fibrinogens removed. Accordingly, serum includes all proteins not used in blood clotting (coagulation) and all the electrolytes, antibodies, antigens, hormones, and any exogenous substances (e.g., drugs and microorganisms). The sample can be diluted with a suitable diluent before contacting the sample to the antibody. Generally, a sample obtained from a subject can be contacted with one or more SARS-CoV-2 proteins, and/or fragments and/or fusions thereof such as those provided herein, e.g., N, M, S, S1, S2′, NSP1, or the amino acids encoded by ORF3a, ORF3b, ORF7a, ORF7b and ORF8 of SARS-CoV-2. Optionally, but preferably, the SARS-CoV-2 protein(s), and/or fragment(s) and/or fusion(s) thereof are fixed to a solid support to facilitate washing and subsequent isolation of the complex, prior to contacting with the biological sample. Examples of solid supports include glass or plastic in the form of, e.g., a microtiter plate, a stick, a bead, or a microbead. SARS-CoV-2 protein(s), and/or fragment(s) and/or fusion(s) thereof can also be attached to a probe substrate or ProteinChip® array and can be analyzed by gas phase ion spectrometry.

Immuoassays for SARS-CoV-2 antibodies can include contacting a biological sample with one or more SARS-CoV-2 proteins, and/or fragments and/or fusions thereof such as those provided herein, e.g., N, M, S, S1, S2′, NSP1, or the amino acids encoded by ORF3a, ORF3b, ORF7a, ORF7b and ORF8 of SARS-CoV-2 under conditions such that an immunospecific antigen-antibody interaction may occur, followed by the detection or measurement of this interaction. The binding of the antibodies to SARS-CoV-2 proteins, or fragments or fusions thereof may be used to detect the presence of SARS-CoV-2 antibodies in the subject from whom the sample was obtained.

An immunoassay can include the steps of detecting and analyzing antibodies in a sample. For example, a method can include the steps of contacting a biological sample with SARS-CoV-2 proteins and/or fragments and/or fusions thereof that can be bound by antibodies, preferably serological antibodies, produced by a subject exposed to or otherwise infected with SARS-CoV-2; and detecting the presence of a complex of the antibodies in the sample bound to the SARS-CoV-2 proteins and/or fragments and/or fusions.

The antibodies can be detected and/or quantified using any of suitable immunological binding assays known in the art. Useful assays include, for example, Luciferase Immunoprecipitation System (LIPS) assay, an enzyme immune assay (EIA) such as enzyme-linked immunosorbent assay (ELISA), a radioimmune assay (RIA), a Western blot assay, or a slot blot assay. See, e.g., Methods in Cell Biology. Antibodies in Cell Biology, volume 37 (Asai, ed. 1993); Basic and Clinical Immunology (Stites & Terr, eds., 7th ed. 1991); and Harlow & Lane, supra.

After incubating the sample with SARS-CoV-2 protein(s), or fragment(s) or fusion(s), the mixture is washed and the antibody-marker complex formed can be detected. This can be accomplished by incubating the washed mixture with a detection reagent. This detection reagent may be, e.g., a second antibody which is labeled with a detectable label. Exemplary detectable labels include magnetic beads (e.g., DYNABEADS™), fluorescent dyes, radiolabels, enzymes (e.g., horse radish peroxide, alkaline phosphatase and others commonly used in an ELISA), and calorimetric labels such as colloidal gold or colored glass or plastic beads. In some embodiments, the antibodies in the sample are detected using an indirect assay, wherein, for example, a second, labeled antibody is used to detect bound SARS-CoV-2 specific antibody, and/or in a competition or inhibition assay wherein, for example, another antibody which binds to a distinct epitope of SARS-CoV-2 proteins, and/or fragments and/or fusions thereof is incubated simultaneously with the mixture.

Exemplary SARS-CoV-2 antibodies are discussed in more detail below.

### A. Immunoprecipitation System (LIPS) Assay

The disclosed compositions can be used in a Luciferase Immunoprecipitation system (LIPS) assay, to detect the presence of the antibody in a sample, and subsequently diagnose a subject as having had exposure to SARS-CoV-2. In one embodiment, a subject is identified as presenting with COVID 19, in an Immunoprecipitation system (LIPS) assay including ORF8, only, as the SARS-CoV-2 antigen. In other embodiments the LIPS assay includes antigens ORF3b and ORF8. In still other embodiments, the LIPS assay includes the sum of N, M, S, S1, S2′, NSP1, ORF3a, ORF3b, ORF7a, ORF7b and ORF8 of SARS-CoV-2

The first step of LIPS involves incubating the serum containing the antibodies and Ruc-antigen lysate together, at room temperature, for 1 h. Although rarely necessary, increased antibody binding can also be achieved by incubation at 4° C. After incubation with sera, the mixture is then transferred to microtiter filter plates containing protein A/G beads for an additional hour to capture both the free immunoglobulins and Ruc-antigen-antibody complexes. For capturing antibodies, high-binding-capacity protein A/G beads (>20 mg of immunoglobulin binding per milliliter of beads) in a microtiter filter plate are used. After incubation of the antigen-antibody complex with protein A/G beads, the filter plate is then extensively rinsed with wash buffer to remove unbound Ruc-tagged antigens. Although manual washing and suctioning using a vacuum manifold can be performed, washing is more easily accomplished with the aid of a robotic workstation or a plate washer with vacuum filtration. After filtration, the filter plate is loaded into a plate luminometer equipped with a substrate injector. Using LIPS, highly quantitative antibody titer values, reported as LUs, can be assigned to clinical and experimental serum samples. From these LIPS tests, a low LU reflects the presence of few or no antibodies, while an elevated LU reflects high antibody titers. Unlike most other immunoassays, the LIPS titer values are often compared using geometric mean titers (GMTs) because of the wide dynamic range and over-dispersed nature of the LIPS data, which are typically represented on a linear to log10 scale depending on the antigen (20).

Thus, a method for detecting antigen-specific antibodies in a biological fluid sample that uses LIPS is provided. In some embodiments, the method includes providing a fusion protein comprising an antigen fused to a light-emitting protein; contacting the biological fluid sample with the fusion protein, If antigen specific antibodies are present in the biological fluid sample, a complex formation of the SARS-CoV-2 antigen fused to light emitting protein with the specific antibody is formed and pulled down using beads. The level of antibodies can then be detected by substrate addition which is proportional to the number of antibodies bound. The beads are preferably non-magnetic, and the method does not include neodymium magnetic sticks.

The light-emitting protein comprises a fluorescent protein or a luciferase, such as a Renilla luciferase, a Gaussia luciferase, a modified (optimized) Oplophorus gracilirostris luciferase (for example, NANOLUC™ (is commercially available), firefly luciferase and bacterial luciferase), a firefly luciferase or a bacterial luciferase. When a luciferase is used as the light-emitting protein, luciferase activity is used as a measure of the quantity of antigen-specific antibody present in the sample. For example, if a Renilla luciferase-antigen fusion protein is used as the light-emitting protein, the antibody is quantified by placing adding coelenterazine and the luciferase activity can be measured, for example, in a luminometer.

In other embodiments, the light-emitting protein comprises a fluorescent protein, such as a green fluorescent protein, a blue fluorescent protein, a cyan fluorescent protein, a yellow fluorescent protein, an orange fluorescent protein, a red fluorescent protein, or a modified version thereof, or a phycobiliprotein, such as B-phycoerythrin (B-PE), R- phycoerythrin (R-PE) or allophycocyanin (APC). When a fluorescent protein is used as the light-emitting protein, fluorescence intensity is used as a measure of the quantity of antigen-specific antibody present in the sample. Fluorescence intensity is measured exposing the bead-bound immune complexes with an appropriate wavelength of light and measuring light emission.

In a preferred embodiment, the light-emitting protein is Renilla luciferase and the substrate is the coelenterazine.

The immunoglobulin-binding protein can be Protein A, Protein G, Protein A/G, Protein L or a secondary immunoglobulin molecule. In a preferred embodiment, the immunoglobulin-binding protein is the protein A/G. In other embodiments, the immunoglobulin-binding protein comprises a secondary antibody, such as anti-IgG antibody, anti-IgM antibody, anti-IgA antibody, anti-IgE antibody, anti-IgD antibody, or any combination or two or more thereof. In particular examples, the secondary antibody comprises anti-IgG antibody. One of skill in the art can select an appropriate immunoglobulin-binding protein based, for example, on the particular immunoglobulin binding properties of each protein antibody.

The biological fluid sample can be any biological fluid in which antibodies can be present. The biological fluid sample can be a serum, plasma, blood, urine, saliva or bronchoalveolar lavage fluid sample, and is preferably, a serum sample.

### B. Other Immunological Assays

Exemplary immunoassays that can be used for the detection of SARS-CoV-2 proteins include, but are not limited to, radioimmunoassays, ELISAs, immunoprecipitation assays, Western blot, fluorescent immunoassays, and immunohistochemistry. In some embodiments, the assay utilize antigens of ORF8 alone, or in combination with ORF3b and/or one or more of M, S, S1, S2′, NSP1, ORF3a, ORF3b, ORF7a, ORF7b and ORF8 of SARS-CoV-2.

A common enzyme immunoassay is the “Enzyme-Linked Immunosorbent Assay (ELISA)”. ELISA is a technique for detecting and measuring the concentration of SARS-CoV-2 antibodies using a labeled (e.g., enzyme linked) form of an antibody. There are different forms of ELISA, which are well known to those skilled in the art. The standard techniques known in the art for ELISA are described in “Methods in Immunodiagnosis”, 2nd Edition, Rose and Bigazzi, eds. John Wiley & Sons, 1980; Campbell et al., “Methods and Immunology”, W. A. Benjamin, Inc., 1964; and Oellerich, M., J. Clin. Chem. Clin. Biochem., 22:895-904 (1984).

For example, in preferred embodiments, the ELISA is an “indirect ELISA.” Indirect ELISAs can include one or more of the following steps. A SARS-CoV-2 protein(s), or fragment(s) or fusion(s) thereof (i.e., the antigen) is added, linked, or otherwise bound to a surface, e.g., each well (usually 96-well plates) of a microtiter plate. In some embodiments, the antigen is added in buffered solution and given time to adhere, e.g., to the plastic through charge interactions or another means.

A solution of nonreacting protein (i.e., blocking agent), such as bovine serum albumin or casein, can be added to each well in order to cover any surface which remains uncoated by the antigen.

The biological sample (which may contain SARS-CoV-2 antibodies) can be added and any antibodies present allowed to specifically bind to the antigen. Unbound sample can be washed out of the well.

A secondary antibody that can detect the presence of the SARS-CoV-2 antibodies (e.g., an antibody specific for a constant region of antibodies produced by subject from which the sample was obtain (i.e., human Ig constant region) with an attached (conjugated) enzyme or other detectable label is added. If required for detection, a substrate for the detectable label (e.g., enzyme) is then added. Often, this substrate changes color upon reaction with the enzyme. In some embodiments, an additional layer is used to further amplify the signal. For example, serum antibodies bind to antigen on the plate. A secondary antibody (e.g., anti-human Ig antibody conjugated to biotin) binds to the serum antibody, and detectable label-linked tertiary ligand binding protein (e.g., avidin-HRP) binds to the secondary antibody. Examples of antibodies that bind to human antibodies that can be used as secondary antibodies include, but are not limited to, antibodies against human Ig, IgG, IgM, IgA, IgG Heavy Chain, IgG Heavy Chain Constant Region, IgG kappa, IgG lambda, IgG Light Chain, IgG1, IgG1 + IgG2 + IgG3 + IgG4, IgG2, IgG2a, IgG2c, IgG3, IgG4, IgGc, or any combination thereof.

The higher concentration of the secondary antibody (or tertiary ligand binding protein) present in the serum, the stronger the color change. Often, a spectrometer is used to give quantitative values for color strength.

The enzyme acts as an amplifier; even if only few enzyme-linked antibodies remain bound, the enzyme molecules will produce many signal molecules. Within common-sense limitations, the enzyme can go on producing color indefinitely, but the more antibody is bound, the faster the color will develop.

In some embodiments, the assay is in a dip stick format. See, e.g., Wu, et al., Clinical and Diagnostic Laboratory Immunology 4(4): 452-7 (1997).

The dipstick can contain SARS-CoV-2 antigen. In some embodiments, antigen is serially diluted (e.g., into an array of dots). The dipstick is contacted with biological sample to allow antibodies present in the sample to bind to the antigen, and can be processed similar to an ELISA. In the dipstick format, the assay can be processed using cuvettes. For example, to detect immunoglobulin G (IgG) the dipstick can be processed through four reaction cuvettes containing biological sample, optionally enhancer, enzyme-conjugated secondary (e.g., anti-human IgG and IgM antibody, etc.), and optionally substrate. To detect IgM, the sample can be passed through a protein G device to remove IgG. The dipstick can then be processed as before for IgG detection.

ELISA may be run in a qualitative or quantitative format. Qualitative results provide a simple positive or negative result (yes or no) for a sample. The cutoff between positive and negative is determined by the analyst and may be statistical. Two or three times the standard deviation (error inherent in a test) is often used to distinguish positive from negative samples. In quantitative ELISA, the optical density (OD) of the sample is compared to a standard curve, which is typically a serial dilution of a known-concentration solution of the target molecule. For example, if a test sample returns an OD of 1.0, the point on the standard curve that gave OD = 1.0 must be of the same analyte concentration as the sample. Other techniques may be used to detect the antibodies, according to a practitioner’s preference, and based upon the present disclosure. One such technique is Western blotting (Towbin et al., Proc. Nat. Acad. Sci. 76:4350 (1979)), wherein a suitably treated sample is run on an SDS-PAGE gel before being transferred to a solid support, such as a nitrocellulose filter. Detectably labeled antibodies that specifically bind to SARS-CoV-2 antibodies can then be used to assess antibody levels, where the intensity of the signal from the detectable label corresponds to the amount of peptide present. Levels can be quantitated, for example by densitometry.

In some embodiments, the assays incorporate one or more methods or reagents that increase the sensitivity or basal detection level of the assays described herein. For example, MESO-SCALE DISCOVERY® (MSD®) technology can be employed to increase sensitivity of an assay. MSD technology includes a combination of electrochemiluminescence detection and patterned arrays. MSD® microplates have electrodes made of carbon integrated into the bottom of the plate. Biological reagents can be attached to the carbon by passive adsorption and retain a high level of biological activity. MSD® assays use electrochemi-luminescent labels for detection. The labels are non-radioactive, stable, a feature a different coupling chemistries. The electrochemiluminescent labels emit light when electrochemically stimulated, which is detected by electrodes in the bottom of the microplates. Only labels near the electrode are excited and detected, so the assay can be performed without washing steps. Additional coreactants are present in the buffers used for the assay. These coreactants are also stimulated when in proximity to the electrodes in the microplate and enhance the electrochemiluminescence signals.

### IV. Detection and Diagnosis

The current or present exposure or infection with SARS-CoV-2 can be detected and/or diagnosed and/or treated using the disclosed compositions and methods. Typically the presence and/or elevated amount of SARS-CoV-2 antibodies in the individual’s biological sample compared to a control is indicative of current or present exposure or infection with SARS-CoV-2 as discussed herein (e.g., above). For example, the method for assisting in the detection or diagnosis of current or present exposure or infection with SARS-CoV-2 in a subject can include determining the presence or level of antibodies against one or more SARS-CoV-2 proteins, fragments, or fusion proteins in a biological sample from the subject, wherein the presence of, or an elevated level of, the antibodies in the biological sample relative to the level of antibodies in a control is indicative of current or present exposure or infection with SARS-CoV-2.

In some embodiments, the method is combined with a method of detecting SARS-CoV-2. If virus is detected, the subject may have a current exposure or infection. If virus is not detected, the subject may not have a current exposure or infection, and the presence of antibodies may be due to a previous exposure or infection.

Any of the methods can be combined with a method of treatment. In preferred embodiments, the method of treatment includes administering the subject an effective amount of an anti-viral therapy, analgesic therapy, fever reducers, cough suppressants, and/or respiratory assistance (e.g., ventilator treatment).

### V. Kits

Assays and kits that include reagents for the detection and qualitative or quantitative measurement of SARS-CoV-2 antibodies in a subject’s biological sample are also provided. For example, if detection of the SARS-CoV-2 antibodies is by means of ELISA, components of the assay or kit can include SARS-CoV-2 protein(s), or fragment(s) or fusion(s) thereof, optional immobilized on a surface such as beads or assay dish or wells thereof, and optionally a detection means such as an antibody that can bind to the SARS-CoV-2 antibody (e.g., an antibody specific for a constant region of antibodies produced by subject from which the sample was obtain (i.e., human IgG constant region) optionally, with an attached (conjugated) enzyme or other detectable label e.g., fluorescent dye or radioactive label.

### EXAMPLES

### Example 1

### Methods

### Patients and Samples Collection

The present study enrolled a total of 26 patients with RT-PCR confirmed COVID-19 infection (Table 1).

TABLE 1

List of ORFs produced as Renilla-Antigen fusion protein

| | | |
|---|---|---|
| Antigen | Full name | Patient response |
| N | Nucleocapsid | Yes |
| M | Membrane | Yes |
| E | Envelope | No |
| S | Spike | Yes |
| S1 | Spike S1 subunit | Yes |
| S2 | Spike S2 subunit | No |
| S2′ | Spike S2 subunit | Yes |
| NSP1 (in ORF1a/b) | - | Yes |
| ORF3a | - | Yes |
| ORF3b | - | Yes |
| ORF6 | - | No |
| ORF7a | - | Yes |
| ORF8 | - | Yes |
| ORF7b | - | Yes |
| ORF10 | - | No |

Fifteen patients were enrolled in Hong Kong (China, SAR) and all of them provided informed consent. The study was approved by the institutional review board of the Hong Kong West Cluster of the Hospital Authority of Hong Kong (approval number: UW20-169). Plasma samples were collected from heparinized blood, and heat-inactivated at 56° C., 30 mins. Gender- and age-matched plasma samples from healthy subjects collected before the COVID-19 pandemic were used as negative controls.

### SARS-CoV-2 Gene Cloning

Based on previous studies describing the structure of the SARS-CoV-2 genome (1, 8), an extensive panel of 12 proteins (S, E, M, N, NSP1, ORF3a, 3b, 6, 7a, 7b, 8, 10) was chosen for antibody testing by LIPS.

The relevant nucleic acids are represented by SEQ ID Nos: 2-16.

Orf3a ATGGATTTGTTTATGAGAATCTTCACAATTGGAACTGTAACTTTGAAGCAA GGTGAAATCAAGGATGCTACTCCTTCAGATTTTGTTCGCGCTACTGCAACG ATACCGATACAAGCCTCACTCCCTTTCGGATGGCTTATTGTTGGCGTTGCA CTTCTTGCTGTTTTTCAGAGCGCTTCCAAAATCATAACCCTCAAAAAGAGA TGGCAACTAGCACTCTCCAAGGGTGTTCACTTTGTTTGCAACTTGCTGTTG TTGTTTGTAACAGTTTACTCACACCTTTTGCTCGTTGCTGCTGGCCTTGAA GCCCCTTTTCTCTATCTTTATGCTTTAGTCTACTTCTTGCAGAGTATAAAC TTTGTAAGAATAATAATGAGGCTTTGGCTTTGCTGGAAATGCCGTTCCAAA AACCCATTACTTTATGATGCCAACTATTTTCTTTGCTGGCATACTAATTGT TACGACTATTGTATACCTTACAATAGTGTAACTTCTTCAATTGTCATTACT TCAGGTGATGGCACAACAAGTCCTATTTCTGAACATGACTACCAGATTGGT GGTTATACTGAAAAATGGGAATCTGGAGTAAAAGACTGTGTTGTATTACAC AGTTACTTCACTTCAGACTATTACCAGCTGTACTCAACTCAATTGAGTACA GACACTGGTGTTGAACATGTTACCTTCTTCATCTACAATAAAATTGTTGAT GAGCCTGAAGAACATGTCCAAATTCACACAATCGACGGTTCATCCGGAGTT GTTAATCCAGTAATGGAACCAATTTATGATGAACCGACGACGACTACTAGC GTGCCTTTGTAA (SEQ ID NO:2);

Orf3b ATGGCTTATTGTTGGCGTTGCACTTCTTGCTGTTTTTCAGAGCGC TTCCAAAATCATAACCCTCAAAAAGAGATGGCAACTAGCACTCTCCAAGG GTGTTCACTTTGTTTGCAACTTGCTGTTGTTGTTTGTAACAGTTTACTCA CACCTTTTGCTCGTTGCTGCTGGCCT (SEQ ID NO:3);

E ATGTACTCATTCGTTTCGGAAGAGACAGGTACGTTAATAGTTAATAGC GTACTTCTTTTTCTTGCTTTCGTGGTATTCTTGCTAGTTACACTAGCCA TCCTTACTGCGCTTCGATTGTGTGCGTACTGCTGCAATATTGTTAACGT GAGTCTTGTAAAACCTTCTTTTTACGTTTACTCTCGTGTTAAAAATCTG AATTCTTCTAGAGTTCCTGATCTTCTGGTCTAA (SEQ ID NO:4);

M ATGGCAGATTCCAACGGTACTATTACCGTTGAAGAGCTTAAAAAGCTCCT TGAACAATGGAACCTAGTAATAGGTTTCCTATTCCTTACATGGATTTGTC TTCTACAATTTGCCTATGCCAACAGGAATAGGTTTTTGTATATAATTAAG TTAATTTTCCTCTGGCTGTTATGGCCAGTAACTTTAGCTTGTTTTGTGCT TGCTGCTGTTTACAGAATAAATTGGATCACCGGTGGAATTGCTATCGCAA TGGCTTGTCTTGTAGGCTTGATGTGGCTCAGCTACTTCATTGCTTCTTTC AGACTGTTTGCGCGTACGCGTTCCATGTGGTCATTCAATCCAGAAACTAA CATTCTTCTCAACGTGCCACTCCATGGCACTATTCTGACCAGACCGCTTC TAGAAAGTGAACTCGTAATCGGAGCTGTGATCCTTCGTGGACATCTTCGT ATTGCTGGACACCATCTAGGACGCTGTGACATCAAGGACCTGCCTAAAGA AATCACTGTTGCTACATCACGAACGCTTTCTTATTACAAATTGGGAGCTT CGCAGCGTGTAGCAGGTGACTCAGGTTTTGCTGCATACAGTCGCTACAGG ATTGGCAACTATAAATTAAACACAGACCATTCCAGTAGCAGTGACAATAT TGCTTTGCTTGTACAGTAA (SEQ ID NO:5);

Orf8 ATGAAATTTCTTGTTTTCTTAGGAATCATCACAACTGTAGCTGCATTTCA CCAAGAATGTAGTTTACAGTCATGTACTCAACATCAACCATATGTAGTTG ATGACCCGTGTCCTATTCACTTCTATTCTAAATGGTATATTAGAGTAGGA GCTAGAAAATCAGCACCTTTAATTGAATTGTGCGTGGATGAGGCTGGTTC TAAATCACCCATTCAGTACATCGATATCGGTAATTATACAGTTTCCTGTT TACCTTTTACAATTAATTGCCAGGAACCTAAATTGGGTAGTCTTGTAGTG CGTTGTTCGTTCTATGAAGACTTTTTAGAGTATCATGACGTTCGTGTTGT TTTAGATTTCATCTAA (SEQ ID NO:6);

N ATGTCTGATAATGGACCCCAAAATCAGCGAAATGCACCCCGCATTACGTT TGGTGGACCCTCAGATTCAACTGGCAGTAACCAGAATGGAGAACGCAGTG GGGCGCGATCAAAACAACGTCGGCCCCAAGGTTTACCCAATAATACTGCG TCTTGGTTCACCGCTCTCACTCAACATGGCAAGGAAGACCTTAAATTCCC TCGAGGACAAGGCGTTCCAATTAACACCAATAGCAGTCCAGATGACCAAA TTGGCTACTACCGAAGAGCTACCAGACGAATTCGTGGTGGTGACGGTAAA ATGAAAGATCTCAGTCCAAGATGGTATTTCTACTACCTAGGAACTGGGCC AGAAGCTGGACTTCCCTATGGTGCTAACAAAGACGGCATCATATGGGTTG CAACTGAGGGAGCCTTGAATACACCAAAAGATCACATTGGCACCCGCAAT CCTGCTAACAATGCTGCAATCGTGCTACAACTTCCTCAAGGAACAACATT GCCAAAAGGCTTCTACGCAGAAGGGAGCAGAGGCGGCAGTCAAGCCTCTT CTCGTTCCTCATCACGTAGTCGCAACAGTTCAAGAAATTCAACTCCAGGC AGCAGTAGGGGAACTTCTCCTGCTAGAATGGCTGGCAATGGCGGTGATGC TGCTCTTGCTTTGCTGCTGCTTGACAGATTGAACCAGCTTGAGAGCAAAA TGTCTGGTAAAGGCCAACAACAACAAGGCCAAACTGTCACTAAGAAATCT GCTGCTGAGGCTTCTAAGAAGCCTCGGCAAAAACGTACTGCCACTAAAGC ATACAATGTAACACAAGCTTTCGGCAGACGTGGTCCAGAACAAACCCAAG GAAATTTTGGGGACCAGGAACTAATCAGACAAGGAACTGATTACAAACAT TGGCCGCAAATTGCACAATTTGCCCCCAGCGCTTCAGCGTTCTTCGGAAT GTCGCGCATTGGCATGGAAGTCACACCTTCGGGAACGTGGTTGACCTACA CAGGTGCCATCAAATTGGATGACAAAGATCCAAATTTCAAAGATCAAGTC ATTTTGCTGAATAAGCATATTGACGCATACAAAACATTCCCACCAACAGA GCCTAAAAAGGACAAAAAGAAGAAGGCTGATGAAACTCAAGCCTTACCGC AGAGACAGAAGAAACAGCAAACTGTGACTCTTCTTCCTGCTGCAGATTTG GATGATTTCTCCAAACAATTGCAACAATCCATGAGCAGTGCTGACTCAAC TCAGGCCTAA (SEQ ID NO:7);

SARS-CoV-2-2019 Spike (full length) ATGTTTGTTTTTCTTGTTTTATTGCCACTAGTCTCTAGTCAGTGTGTTAA TCTTACAACCAGAACTCAATTACCCCCTGCATACACTAATTCTTTCACAC GTGGTGTTTATTACCCTGACAAAGTTTTCAGATCCTCAGTTTTACATTCA ACTCAGGACTTGTTCTTACCTTTCTTTTCCAATGTTACTTGGTTCCATGC TATACATGTCTCTGGGACCAATGGTACTAAGAGGTTTGATAACCCTGTCC TACCATTTAATGATGGTGTTTATTTTGCTTCCACTGAGAAGTCTAACATA ATAAGAGGCTGGATTTTTGGTACTACTTTAGATTCGAAGACCCAGTCCCT ACTTATTGTTAATAACGCTACTAATGTTGTTATTAAAGTCTGTGAATTTC AATTTTGTAATGATCCATTTTTGGGTGTTTATTACCACAAAAACAACAAA AGTTGGATGGAAAGTGAGTTCAGAGTTTATTCTAGTGCGAATAATTGCAC TTTTGAATATGTCTCTCAGCCTTTTCTTATGGACCTTGAAGGAAAACAGG GTAATTTCAAAAATCTTAGGGAATTTGTGTTTAAGAATATTGATGGTTAT TTTAAAATATATTCTAAGCACACGCCTATTAATTTAGTGCGTGATCTCCC TCAGGGTTTTTCGGCTTTAGAACCATTGGTAGATTTGCCAATAGGTATTA ACATCACTAGGTTTCAAACTTTACTTGCTTTACATAGAAGTTATTTGACT CCTGGTGATTCTTCTTCAGGTTGGACAGCTGGTGCTGCAGCTTATTATGT GGGTTATCTTCAACCTAGGACTTTTCTATTAAAATATAATGAAAATGGAA CCATTACAGATGCTGTAGACTGTGCACTTGACCCTCTCTCAGAAACAAAG TGTACGTTGAAATCCTTCACTGTAGAAAAAGGAATCTATCAAACTTCTAA CTTTAGAGTCCAACCAACAGAATCTATTGTTAGATTTCCTAATATTACAA ACTTGTGCCCTTTTGGTGAAGTTTTTAACGCCACCAGATTTGCATCTGTT TATGCTTGGAACAGGAAGAGAATCAGCAACTGTGTTGCTGATTATTCTGT CCTATATAATTCCGCATCATTTTCCACTTTTAAGTGTTATGGAGTGTCTC CTACTAAATTAAATGATCTCTGCTTTACTAATGTCTATGCAGATTCATTT GTAATTAGAGGTGATGAAGTCAGACAAATCGCTCCAGGGCAAACTGGAAA GATTGCTGATTATAATTATAAATTACCAGATGATTTTACAGGCTGCGTTA TAGCTTGGAATTCTAACAATCTTGATTCTAAGGTTGGTGGTAATTATAAT TACCTGTATAGATTGTTTAGGAAGTCTAATCTCAAACCTTTTGAGAGAGA TATTTCAACTGAAATCTATCAGGCCGGTAGCACACCTTGTAATGGTGTTG AAGGTTTTAATTGTTACTTTCCTTTACAATCATATGGTTTCCAACCCACT AATGGTGTTGGTTACCAACCATACAGAGTAGTAGTACTTTCTTTTGAACT TCTACATGCACCAGCAACTGTTTGTGGACCTAAAAAGTCTACTAATTTGG TTAAAAACAAATGTGTCAATTTCAACTTCAATGGTTTAACAGGCACAGGT GTTCTTACTGAGTCTAACAAAAAGTTTCTGCCTTTCCAACAATTTGGCAG AGACATTGCTGACACTACTGATGCTGTCCGTGATCCACAGACACTTGAGA TTCTTGACATTACACCATGTTCTTTTGGTGGTGTCAGTGTTATAACACCA GGAACAAATACTTCTAACCAGGTTGCTGTTCTTTATCAGGATGTTAACTG CACAGAAGTCCCTGTTGCTATTCATGCAGATCAACTTACTCCTACTTGGC GTGTTTATTCTACAGGTTCTAATGTTTTTCAAACACGTGCAGGCTGTTTA ATAGGGGCTGAACATGTCAACAACTCATATGAGTGTGACATACCCATTGG TGCAGGTATATGCGCTAGTTATCAGACTCAGACTAATTCTCCTCGGCGGG CACGTAGTGTAGCTAGTCAATCCATCATTGCCTACACTATGTCACTTGGT GCAGAAAATTCAGTTGCTTACTCTAATAACTCTATTGCCATACCCACAAA TTTTACTATTAGTGTTACCACAGAAATTCTACCAGTGTCTATGACCAAGA CATCAGTAGATTGTACAATGTACATTTGTGGTGATTCAACTGAATGCAGC AATCTTTTGTTGCAATATGGCAGTTTTTGTACACAATTAAACCGTGCTTT AACTGGAATAGCTGTTGAACAAGACAAAAACACCCAAGAAGTTTTTGCAC AAGTCAAACAAATTTACAAAACACCACCAATTAAAGATTTTGGTGGTTTT AATTTTTCACAAATATTACCAGATCCATCAAAACCAAGCAAGAGGTCATT TATTGAAGATCTACTTTTCAACAAAGTGACACTTGCAGATGCTGGCTTCA TCAAACAATATGGTGATTGCCTTGGTGATATTGCTGCTAGAGACCTCATT TGTGCACAAAAGTTTAACGGCCTTACTGTTTTGCCACCTTTGCTCACAGA TGAAATGATTGCTCAATACACTTCTGCACTGTTAGCGGGTACAATCACTT CTGGTTGGACCTTTGGTGCAGGTGCTGCATTACAAATACCATTTGCTATG CAAATGGCTTATAGGTTTAATGGTATTGGAGTTACACAGAATGTTCTCTA TGAGAACCAAAAATTGATTGCCAACCAATTTAATAGTGCTATTGGCAAAA TTCAAGACTCACTTTCTTCCACAGCAAGTGCACTTGGAAAACTTCAAGAT GTGGTCAACCAAAATGCACAAGCTTTAAACACGCTTGTTAAACAACTTAG CTCCAATTTTGGTGCAATTTCAAGTGTTTTAAATGATATCCTTTCACGTC TTGACAAAGTTGAGGCTGAAGTGCAAATTGATAGGTTGATCACAGGCAGA CTTCAAAGTTTGCAGACATATGTGACTCAACAATTAATTAGAGCTGCAGA AATCAGAGCTTCTGCTAATCTTGCTGCTACTAAAATGTCAGAGTGTGTAC TTGGACAATCAAAAAGAGTTGATTTTTGTGGAAAGGGCTATCATCTTATG TCCTTCCCTCAGTCAGCACCTCATGGTGTAGTCTTCTTGCATGTGACTTA TGTCCCTGCACAAGAAAAGAACTTCACAACTGCTCCTGCCATTTGTCATG ATGGAAAAGCACACTTTCCTCGTGAAGGTGTCTTTGTTTCAAATGGCACA CACTGGTTTGTAACACAAAGGAATTTTTATGAACCACAAATCATTACTAC AGACAACACATTTGTGTCTGGTAACTGTGATGTTGTAATAGGAATTGTCA ACAACACAGTTTATGATCCTTTGCAACCTGAATTAGACTCATTCAAGGAG GAGTTAGATAAATATTTTAAGAATCATACATCACCAGATGTTGATTTAGG TGACATCTCTGGCATTAATGCTTCAGTTGTAAACATTCAAAAAGAAATTG ACCGCCTCAATGAGGTTGCCAAGAATTTAAATGAATCTCTCATCGATCTC CAAGAACTTGGAAAGTATGAGCAGTATATAAAATGGCCATGGTACATTTG GCTAGGTTTTATAGCTGGCTTGATTGCCATAGTAATGGTGACAATTATGC TTTGCTGTATGACCAGTTGCTGTAGTTGTCTCAAGGGCTGTTGTTCTTGT GGATCCTGCTGCAAATTTGATGAAGACGACTCTGAGCCAGTGCTCAAAGG AGTCAAATTACATTACACATAA (SEQ ID NO:8).

S1 (8) ATGTGTGTTAATCTTACAACCAGAACTCAATTACCCCCTGCATACACTAA TTCTTTCACACGTGGTGTTTATTACCCTGACAAAGTTTTCAGATCCTCAG TTTTACATTCAACTCAGGACTTGTTCTTACCTTTCTTTTCCAATGTTACT TGGTTCCATGCTATACATGTCTCTGGGACCAATGGTACTAAGAGGTTTGA TAACCCTGTCCTACCATTTAATGATGGTGTTTATTTTGCTTCCACTGAGA AGTCTAACATAATAAGAGGCTGGATTTTTGGTACTACTTTAGATTCGAAG ACCCAGTCCCTACTTATTGTTAATAACGCTACTAATGTTGTTATTAAAGT CTGTGAATTTCAATTTTGTAATGATCCATTTTTGGGTGTTTATTACCACA AAAACAACAAAAGTTGGATGGAAAGTGAGTTCAGAGTTTATTCTAGTGCG AATAATTGCACTTTTGAATATGTCTCTCAGCCTTTTCTTATGGACCTTGA AGGAAAACAGGGTAATTTCAAAAATCTTAGGGAATTTGTGTTTAAGAATA TTGATGGTTATTTTAAAATATATTCTAAGCACACGCCTATTAATTTAGTG CGTGATCTCCCTCAGGGTTTTTCGGCTTTAGAACCATTGGTAGATTTGCC AATAGGTATTAACATCACTAGGTTTCAAACTTTACTTGCTTTACATAGAA GTTATTTGACTCCTGGTGATTCTTCTTCAGGTTGGACAGCTGGTGCTGCA GCTTATTATGTGGGTTATCTTCAACCTAGGACTTTTCTATTAAAATATAA TGAAAATGGAACCATTACAGATGCTGTAGACTGTGCACTTGACCCTCTCT CAGAAACAAAGTGTACGTTGAAATCCTTCACTGTAGAAAAAGGAATCTAT CAAACTTCTAACTTTAGAGTCCAACCAACAGAATCTATTGTTAGATTTCC TAATATTACAAACTTGTGCCCTTTTGGTGAAGTTTTTAACGCCACCAGAT TTGCATCTGTTTATGCTTGGAACAGGAAGAGAATCAGCAACTGTGTTGCT GATTATTCTGTCCTATATAATTCCGCATCATTTTCCACTTTTAAGTGTTA TGGAGTGTCTCCTACTAAATTAAATGATCTCTGCTTTACTAATGTCTATG CAGATTCATTTGTAATTAGAGGTGATGAAGTCAGACAAATCGCTCCAGGG CAAACTGGAAAGATTGCTGATTATAATTATAAATTACCAGATGATTTTAC AGGCTGCGTTATAGCTTGGAATTCTAACAATCTTGATTCTAAGGTTGGTG GTAATTATAATTACCTGTATAGATTGTTTAGGAAGTCTAATCTCAAACCT TTTGAGAGAGATATTTCAACTGAAATCTATCAGGCCGGTAGCACACCTTG TAATGGTGTTGAAGGTTTTAATTGTTACTTTCCTTTACAATCATATGGTT TCCAACCCACTAATGGTGTTGGTTACCAACCATACAGAGTAGTAGTACTT TCTTTTGAACTTCTACATGCACCAGCAACTGTTTGTGGACCTAAAAAGTC TACTAATTTGGTTAAAAACAAATGTGTCAATTTCAACTTCAATGGTTTAA CAGGCACAGGTGTTCTTACTGAGTCTAACAAAAAGTTTCTGCCTTTCCAA CAATTTGGCAGAGACATTGCTGACACTACTGATGCTGTCCGTGATCCACA GACACTTGAGATTCTTGACATTACACCATGTTCTTTTGGTGGTGTCAGTG TTATAACACCAGGAACAAATACTTCTAACCAGGTTGCTGTTCTTTATCAG GATGTTAACTGCACAGAAGTCCCTGTTGCTATTCATGCAGATCAACTTAC TCCTACTTGGCGTGTTTATTCTACAGGTTCTAATGTTTTTCAAACACGTG CAGGCTGTTTAATAGGGGCTGAACATGTCAACAACTCATATGAGTGTGAC ATACCCATTGGTGCAGGTATATGCGCTAGTTATCAGACTCAGACTAATTC TCCTCGGCGGGCA (SEQ ID NO:9);

S2 (8) ATGCGTAGTGTAGCTAGTCAATCCATCATTGCCTACACTATGTCACTTGG TGCAGAAAATTCAGTTGCTTACTCTAATAACTCTATTGCCATACCCACAA ATTTTACTATTAGTGTTACCACAGAAATTCTACCAGTGTCTATGACCAAG ACATCAGTAGATTGTACAATGTACATTTGTGGTGATTCAACTGAATGCAG CAATCTTTTGTTGCAATATGGCAGTTTTTGTACACAATTAAACCGTGCTT TAACTGGAATAGCTGTTGAACAAGACAAAAACACCCAAGAAGTTTTTGCA CAAGTCAAACAAATTTACAAAACACCACCAATTAAAGATTTTGGTGGTTT TAATTTTTCACAAATATTACCAGATCCATCAAAACCAAGCAAGAGGTCAT TTATTGAAGATCTACTTTTCAACAAAGTGACACTTGCAGATGCTGGCTTC ATCAAACAATATGGTGATTGCCTTGGTGATATTGCTGCTAGAGACCTCAT TTGTGCACAAAAGTTTAACGGCCTTACTGTTTTGCCACCTTTGCTCACAG ATGAAATGATTGCTCAATACACTTCTGCACTGTTAGCGGGTACAATCACT TCTGGTTGGACCTTTGGTGCAGGTGCTGCATTACAAATACCATTTGCTAT GCAAATGGCTTATAGGTTTAATGGTATTGGAGTTACACAGAATGTTCTCT ATGAGAACCAAAAATTGATTGCCAACCAATTTAATAGTGCTATTGGCAAA ATTCAAGACTCACTTTCTTCCACAGCAAGTGCACTTGGAAAACTTCAAGA TGTGGTCAACCAAAATGCACAAGCTTTAAACACGCTTGTTAAACAACTTA GCTCCAATTTTGGTGCAATTTCAAGTGTTTTAAATGATATCCTTTCACGT CTTGACAAAGTTGAGGCTGAAGTGCAAATTGATAGGTTGATCACAGGCAG ACTTCAAAGTTTGCAGACATATGTGACTCAACAATTAATTAGAGCTGCAG AAATCAGAGCTTCTGCTAATCTTGCTGCTACTAAAATGTCAGAGTGTGTA CTTGGACAATCAAAAAGAGTTGATTTTTGTGGAAAGGGCTATCATCTTAT GTCCTTCCCTCAGTCAGCACCTCATGGTGTAGTCTTCTTGCATGTGACTT ATGTCCCTGCACAAGAAAAGAACTTCACAACTGCTCCTGCCATTTGTCAT GATGGAAAAGCACACTTTCCTCGTGAAGGTGTCTTTGTTTCAAATGGCAC ACACTGGTTTGTAACACAAAGGAATTTTTATGAACCACAAATCATTACTA CAGACAACACATTTGTGTCTGGTAACTGTGATGTTGTAATAGGAATTGTC AACAACACAGTTTATGATCCTTTGCAACCTGAATTAGACTCATTCAAGGA GGAGTTAGATAAATATTTTAAGAATCATACATCACCAGATGTTGATTTAG GTGACATCTCTGGCATTAATGCTTCAGTTGTAAACATTCAAAAAGAAATT GACCGCCTCAATGAGGTTGCCAAGAATTTAAATGAATCTCTCATCGATCT CCAAGAACTTGGAAAGTATGAGCAGTATATAAAATGGCCATGGTACATTT GGCTAGGTTTTATAGCTGGCTTGATTGCCATAGTAATGGTGACAATTATG CTTTGCTGTATGACCAGTTGCTGTAGTTGTCTCAAGGGCTGTTGTTCTTG TGGATCCTGCTGCAAATTTGATGAAGACGACTCTGAGCCAGTGCTCAAAG GAGTCAAATTACATTACACA(SEQ ID NO:10);

S2’ (8) ATGAGGTCATTTATTGAAGATCTACTTTTCAACAAAGTGACACTTGCAGA TGCTGGCTTCATCAAACAATATGGTGATTGCCTTGGTGATATTGCTGCTA GAGACCTCATTTGTGCACAAAAGTTTAACGGCCTTACTGTTTTGCCACCT TTGCTCACAGATGAAATGATTGCTCAATACACTTCTGCACTGTTAGCGGG TACAATCACTTCTGGTTGGACCTTTGGTGCAGGTGCTGCATTACAAATAC CATTTGCTATGCAAATGGCTTATAGGTTTAATGGTATTGGAGTTACACAG AATGTTCTCTATGAGAACCAAAAATTGATTGCCAACCAATTTAATAGTGC TATTGGCAAAATTCAAGACTCACTTTCTTCCACAGCAAGTGCACTTGGAA AACTTCAAGATGTGGTCAACCAAAATGCACAAGCTTTAAACACGCTTGTT AAACAACTTAGCTCCAATTTTGGTGCAATTTCAAGTGTTTTAAATGATAT CCTTTCACGTCTTGACAAAGTTGAGGCTGAAGTGCAAATTGATAGGTTGA TCACAGGCAGACTTCAAAGTTTGCAGACATATGTGACTCAACAATTAATT AGAGCTGCAGAAATCAGAGCTTCTGCTAATCTTGCTGCTACTAAAATGTC AGAGTGTGTACTTGGACAATCAAAAAGAGTTGATTTTTGTGGAAAGGGCT ATCATCTTATGTCCTTCCCTCAGTCAGCACCTCATGGTGTAGTCTTCTTG CATGTGACTTATGTCCCTGCACAAGAAAAGAACTTCACAACTGCTCCTGC CATTTGTCATGATGGAAAAGCACACTTTCCTCGTGAAGGTGTCTTTGTTT CAAATGGCACACACTGGTTTGTAACACAAAGGAATTTTTATGAACCACAA ATCATTACTACAGACAACACATTTGTGTCTGGTAACTGTGATGTTGTAAT AGGAATTGTCAACAACACAGTTTATGATCCTTTGCAACCTGAATTAGACT CATTCAAGGAGGAGTTAGATAAATATTTTAAGAATCATACATCACCAGAT GTTGATTTAGGTGACATCTCTGGCATTAATGCTTCAGTTGTAAACATTCA AAAAGAAATTGACCGCCTCAATGAGGTTGCCAAGAATTTAAATGAATCTC TCATCGATCTCCAAGAACTTGGAAAGTATGAGCAGTATATAAAATGGCCA TGGTACATTTGGCTAGGTTTTATAGCTGGCTTGATTGCCATAGTAATGGT GACAATTATGCTTTGCTGTATGACCAGTTGCTGTAGTTGTCTCAAGGGCT GTTGTTCTTGTGGATCCTGCTGCAAATTTGATGAAGACGACTCTGAGCCA GTGCTCAAAGGAGTCAAATTACATTACACA (SEQ ID NO:11);

Nsp1 ATGGAGAGCCTTGTCCCTGGTTTCAACGAGAAAACACACGTCCAACTCAG TTTGCCTGTTTTACAGGTTCGCGACGTGCTCGTACGTGGCTTTGGAGACT CCGTGGAGGAGGTCTTATCAGAGGCACGTCAACATCTTAAAGATGGCACT TGTGGCTTAGTAGAAGTTGAAAAAGGCGTTTTGCCTCAACTTGAACAGCC CTATGTGTTCATCAAACGTTCGGATGCTCGAACTGCACCTCATGGTCATG TTATGGTTGAGCTGGTAGCAGAACTCGAAGGCATTCAGTACGGTCGTAGT GGTGAGACACTTGGTGTCCTTGTCCCTCATGTGGGCGAAATACCAGTGGC TTACCGCAAGGTTCTTCTTCGTAAGAACGGTAATAAAGGAGCTGGTGGCC ATAGTTACGGCGCCGATCTAAAGTCATTTGACTTAGGCGACGAGCTTGGC ACTGATCCTTATGAAGATTTTCAAGAAAACTGGAACACTAAACATAGCAG TGGTGTTACCCGTGAACTCATGCGTGAGCTTAACGGAGGG (SEQ ID NO:12);

Orf6 ATGTTTCATCTCGTTGACTTTCAGGTTACTATAGCAGAGATATTACTAAT TATTATGAGGACTTTTAAAGTTTCCATTTGGAATCTTGATTACATCATAA ACCTCATAATTAAAAATTTATCTAAGTCACTAACTGAGAATAAATATTCT CAATTAGATGAAGAGCAACCAATGGAGATTGATTAA (SEQ ID NO:13);

Orf7 aATGAAAATTATTCTTTTCTTGGCACTGATAACACTCGCTACTTGTGAGC TTTATCACTACCAAGAGTGTGTTAGAGGTACAACAGTACTTTTAAAAGAA CCTTGCTCTTCTGGAACATACGAGGGCAATTCACCATTTCATCCTCTAGC TGATAACAAATTTGCACTGACTTGCTTTAGCACTCAATTTGCTTTTGCTT GTCCTGACGGCGTAAAACACGTCTATCAGTTACGTGCCAGATCAGTTTCA CCTAAACTGTTCATCAGACAAGAGGAAGTTCAAGAACTTTACTCTCCAAT TTTTCTTATTGTTGCGGCAATAGTGTTTATAACACTTTGCTTCACACTCA AAAGAAAGACAGAATGA (SEQ ID NO:14);

Orf7b ATGATTGAACTTTCATTAATTGACTTCTATTTGTGCTTTTTAGCCTTTCTG CTATTCCTTGTTTTAATTATGCTTATTATCTTTTGGTTCTCACTTGAACTG CAAGATCATAATGAAACTTGTCACGCCTAA (SEQ ID NO:15);

Orf10 ATGGGCTATATAAACGTTTTCGCTTTTCCGTTTACGATATATAGTCTACTC TTGTGCAGAATGAATTCTCGTAACTACATAGCACAAGTAGATGTAGTTAAC TTTAATCTCACATAG (SEQ ID NO: 16)

Primers for the amplification of SARS-CoV-2 proteins were designed (See protein Sequence ID and primers sequences in table 2).

TABLE 2

SARS-CoV-2 proteins and primers for LIPS

| | | | |
|---|---|---|---|
| Gene | Sequence ID | Forward primer | Reverse primer |
| NSP1 (ORF1a /b) | QIA20043.1 | CTGAGCGGATCCATGGAGAGCCTTGTCCCTG (SEQ ID NO:17) | CTGAGCGGCCGCCCCTCCGTTAAGCTCACG (SEQ ID NO:18) |
| S | QHD43416.1 | CTGACTCGAGATGTTTGTTTTTCTTGTTTTATTGC (SEQ ID NO:19) | CTGAGGTACCTTATGTGTAATGTAATTTGACTCC (SEQ ID NO:20) |
| S1 | QHD43416.1 | CTGACTCGAGATGTGTGTTAATCTT (SEQ ID NO:21) | CTGAGGTACCTGCCCGCCGAGGAGAATTA (SEQ ID NO:22) |
| S2 | QHD43416.1 | CTGACTCGAGATGCGTAGTGTAGCTAGTCAAT (SEQ ID NO:23) | CTGAGGTACCTGTGTAATGTAATTTGACTC (SEQ ID NO:24) |
| S2′ | QHD43416.1 | CTGACTCGAGATGAGGTCATTTATTGAAGA (SEQ ID NO:25) | CTGAGGTACCTGTGTAATGTAATTTGACTC (SEQ ID NO:26) |
| NP | QHD43423.2 | CTGAGCGGATCCATGTCTGATAATGGACC (SEQ ID NO:27) | CTGAGCGGCCGCTTAGGCCTGAGTTGAGTCAG (SEQ ID NO:28) |
| E | QHD43418.1 | CTGAGCGGATCCTGTACTCATTCGTTTCGGAAGA (SEQ ID NO:29) | CTGAGCGGCCGCGACCAGAAGATCAGGAACTC (SEQ ID NO:30) |
| M | QHD43419.1 | CTGAGCGGATCCATGGCAGATTCCAACGGTACT (SEQ ID NO:31) | CTGAGCGGCCGCGCAAAGCAATATTGTCACTGCTAC (SEQ ID NO:32) |
| ORF3a | QHD43417.1 | CTGAGCGGATCCATGGATTTGTTTATGAG (SEQ ID NO:33) | CTGAGCGGCCGCTTACAAAGGCACGCTAGTAGTC (SEQ ID NO:34) |
| ORF3b | PMID319870 01 | CTGAGCGGATCCATGGCTTATTGTTGGCG (SEQ ID NO:35) | CTGAGCGGCCGCAGGCCAGCAGCAACGAG (SEQ ID NO:36) |
| ORF6 | QHD43420.1 | CTGAGCGGATCCATGTTTCATCTCGTTG (SEQ ID NO:37) | CTGAGCGGCCGCTTAATCAATCTCCATTG (SEQ ID NO:38) |
| ORF7a | QHD43421.1 | CTGAGCGGATCCATGAAAATTATTCTT (SEQ ID NO:39) | CTGAGCGGCCGCTCATTCTGTCTTTCTTT (SEQ ID NO:40) |
| ORF7b | QIA20050.1 | CTGAGCGGATCCATGATTGAACTTTCATT (SEQ ID NO:41) | CTGAGCGGCCGCTTAGGCGTGACAAGTTTC (SEQ ID NO:42) |
| ORF8 | QHD43422.1 | CTGAGCGGATCCATGAAATTTCTTGTTTTC (SEQ ID NO:43) | CTGAGCGGCCGCTTAGATGAAATCTAAAAC (SEQ ID NO:44) |
| ORF10 | QHI42199.1 | CTGAGCGGATCCATGGGCTATATAAACGT ((SEQ ID NO:45) | CTGAGCGGCCGCCTATGTGAGATTAAAGT ((SEQ ID NO:1) |

Information on the COVID 19 patients is provided in Table 3.

TABLE 3

Hong Kong COVID-19 infected patient information

| Patient number | gender | age | sample day | SARS-CoV-2 MN titers |
|---|---|---|---|---|
| 1 2 3 4 5 6 7 8 9 10 11 12 13 14 15 | M F M F M M F F F M F M M F M | 56 62 47 72 56 72 37 73 55 64 63 60 37 62 63 | 6 4 8 10 13 18 15 18 11 19 19 22 14 22 22 | <1:10 <1:10 1/80 1/40 1/320 1/80 1/80 1/80 <1:10 1/320 1/640 1/160 1/320 1/160 1/80 |

A RT-PCR was performed using extracted SARS-CoV-2 vRNA to amplify target genes corresponding to Structural and Non-structural proteins of the virus (Table 1, (6)) using Platinum SuperScriptIII One Step RT-PCR system. The bands were then extracted using Qiagen gel extraction kit (Qiagen, Germany) and digested with BamHI and NotI or KpnI-HF and XhoI (New England Biolabs, USA). Extracted products were ligated using T4 DNA ligase (New England Biolabs) into the pREN2 plasmid (from Peter Burbelo NDICR, NIH). Plasmids were transformed using DH10B competent cells and purified using PureYield Plasmid mid-prep system (Promega). All constructs were confirmed by Sanger Sequencing (3730x1 DNA Analyzer Applied Biosystems).

### SARS-CoV-2 (Ruc)-Antigen Expression

Constructs with pREN2-Renilla luciferase plasmid containing the SARS-CoV-2 antigen of interest were transfected into Cos1 cells using Fugene 6 (Promega) as per manufacturer’s instructions. Cells were harvested 48 hours later, lysed and sonicated, and (Ruc)-antigen yields were measured using a Luminometer plate reader (PerkinElmer) according to the protocol of Burbelo et al. (20).

### Measurement of Antibody Responses Using Luciferase Immunoprecipitation System (LIPS)

The LIPS assays were performed following the protocol of Burbelo et al., with the following modifications (20). Briefly, (Ruc)-antigen (1e7 per well) and plasma (heat inactivated and diluted 1:100) were incubated for 2 hours with shaking at 800 rpm. Ultralink protein A/G beads were added to the (Ruc)-antigen and serum mixture in a 96-deep-well polypropylene microtiter plate and incubated for 2 hours with shaking at 800 rpm. The entire volume was then transferred into HTS plates and washed as previously described. The plate was read using QUANTI-Luc™ Gold substrate (Invivogen, rep-qlcg5) as per manufacturer’s instructions and a MicroBeta JET luminometer (PerkinElmer). UANTI-Luc™ Gold is an optimized kit for the detection of Lucia luciferase and other coelenterazine-utilizing luciferases (e.g. Gaussia and Renilla luciferases). The light signal produced by the luciferase reaction with the coelenterazine substrate is quantified using a luminometer and expressed as relative light units (RLU). Experimental controls include blank wells with antigens and negative control serum from age matched non-infected patient plasma collected prior to the COVID-19 pandemic.

### Enzyme-Linked Immunosorbent Assay

ELISA assays were performed with the available SARS-CoV-2 proteins Spike (S1+S2) and Nucleoprotein (N) proteins. Briefly, recombinant S and N proteins (Sinobiological) were coated on 96-well flat-bottom immunosorbent plates (Nunc Immuno MaxiSorp, Roskilde, Denmark) at a concentration of 80 ng/ml, in 100/µl coating buffer (PBS with 53% Na₂CO₃ and 42% NaHCO₃, pH 9.6) at 4° C. overnight. An additional plate coated with a non-specific protein (blocking buffer, PBS with 5% FBS) was used to measure the background binding of each sample. Following FBS blocking and thorough washing, diluted plasma samples (1:100) were bound for 2 hours, further washed and then detected by an anti-human Ig secondary antibody labelled with HRP specific for IgG (Invitrogen, Carlsbad, CA, USA).

### Microneutralization Assay

Plasma samples were diluted in serial two-fold dilutions commencing with a dilution of 1:10, and mixed with equal volumes of SARS-CoV-2 at a dose of 200 tissue culture infective doses 50% (TCID₅₀) determined by Vero E6 cells respectively. After 1 hour of incubation at 37° C., 35 µl of the virus-serum mixture was added in quadruplicate to Vero or Vero E6 cell monolayers in 96-well microtiter plates. After 1 hour of adsorption, the virus-serum mixture was removed and replaced with 150 µl of virus growth medium in each well. The plates were incubated for 3 days at 37° C. in 5% CO₂ in a humidified incubator. Cytopathic effect was observed at day 3 post-inoculation. The highest plasma dilution protecting 50% of the replicate wells was denoted as the neutralizing antibody titer. A virus back-titration of the input virus was included in each batch of tests.

### Multiple Alignments of Coronaviruses

The multiple amino acid alignments of HKU1 (AY597011.2), HCoV-229E (AF304460.1), HCoV-OC43 (AY391777.1) and HCoV-NL63 (AY567487.2) versus SARS-CoV-2 (MN908947) were performed using CLUSTAL 2.1.

### Statistics

GraphPad Prism 6 software (San Diego, CA) was used for statistical analysis. Antibody levels are presented as the geometric mean +/- standard deviation (stdev). For the calculation of sensitivity and specificity, cut-off limits for each antigen were derived from the mean value plus three standard deviations of the controls. Non-parametric Mann-Whitney U tests were used to compare the antibody levels between COVID-19 and negative groups, using the GraphPad 8 Prism software.

### Results

A panel of fifteen SARS-CoV-2 ORFs were made as Renilla luciferase-antigen fusion proteins to assess the humoral immune responses in 15 COVID-19 infected patients from Hong Kong compared with a panel of healthy negative controls using LIPS. The four SARS-CoV-2 structural proteins show high amino-acid sequence homology with SARS-CoV but not with other human coronaviruses responsible for common colds (Table 4).

TABLE 4

% amino acid sequence homology of SARS-CoV-2 structural proteins versus other human coronaviruses

| SARS-CoV-2 vs Gene | SARS-CoV* | HKU1 | HCoV-E229 | HCoV-NL63 | HCoV-OC43 |
|---|---|---|---|---|---|
| N M E S ORF1a/b | 94 91 95 76 90 | 34 37 30 33 33 | 29 32 28 31 40 | 31 31 18 29 42 | 39 40 27 34 45 |

* NB: From (8) Chan JF, Kok KH, Zhu Z, Chu H, To KK, Yuan S, et al. Emerg Microbes Infect. 2020;9(1):221-3.

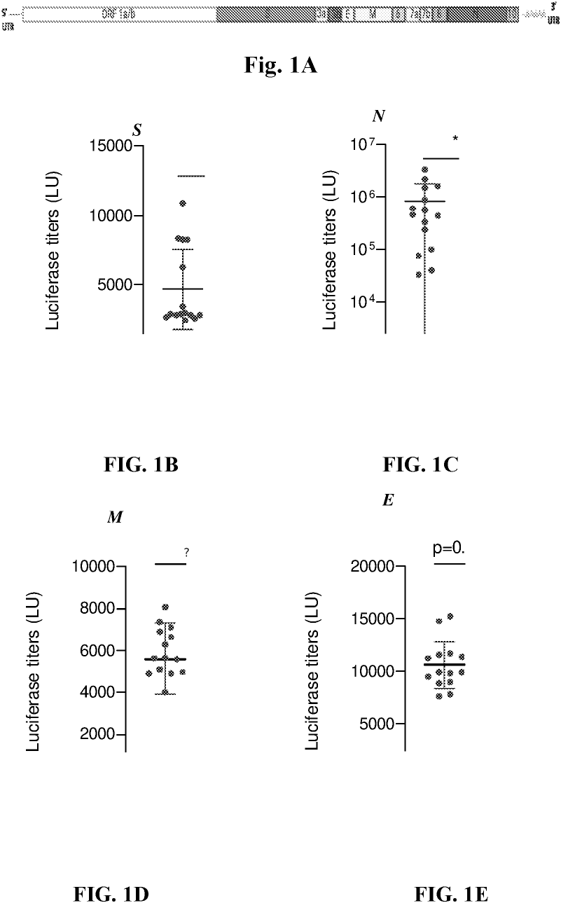

Significantly higher antibody responses to the full Spike protein (S), the Nucleoprotein (N) and the Membrane protein (M) were detected in COVID-19 patients compared to healthy controls (p <0.0001, p=0.023, and p=0.0116 respectively, FIGS. 1B-D). COVID-19 patients did not show any increased production of Envelope antibodies (FIG. 1E) compared to healthy controls (p=0.0591, FIG. 1D).

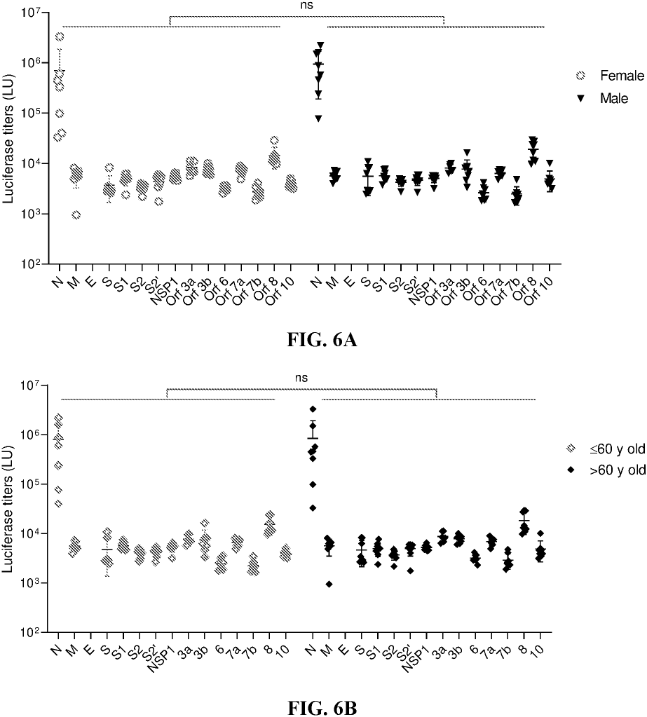

The results were confirmed by traditional IgG ELISA, which correlated well with LIPS LU titers (R2=0.5289) for full-S (FIGS. 6A and B). N-specific IgG by ELISA is also elevated in COVID-19 patients (FIG. 6C) but does not correlate strongly with LIPS results (FIG. 6D), but a similar antigenic region is bound following confirmation by immune-absorbance to N protein (FIG. 6E).

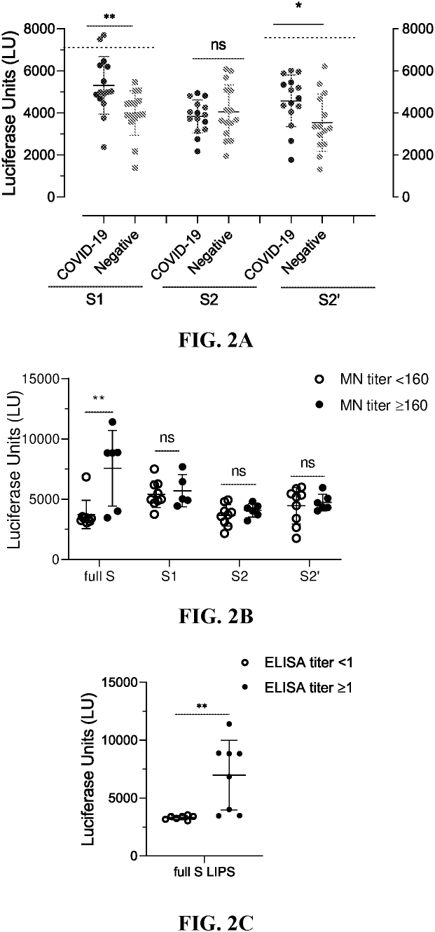

The S1 subunit, a key virus immunogen, detected significantly higher antibodies in COVID-19 patients than healthy controls (5191+/-1469 LU versus 4003+/-1062 LU, p=0.0288, FIG. 2A). Interestingly, there was no difference in the antibodies to S2 in the LIPS assay between COVID-19 patients and healthy controls (p=0.5683). Antibodies to the S2′ cleaved subunit were significantly higher in COVID-19 patients (p=0.0391 for S2′, FIGS. 2A-B) but the overall in antibody levels between the groups was considerable. Patients with higher SARS-CoV-2 micro neutralization (MN) titers (>160 reciprocal serum dilution) also had higher responses towards full-S by LIPS, whilst there was no difference observed in LIPS responses to subunits S1, S2 and S2′ in high or low MN COVID-19 patients (FIG. 2B, p=0.0049), but subjects with higher S responses by ELISA also had higher S responses by LIPS (FIG. 2C)..

The presence of antibodies specific to previously uncharacterized ORFS to the SARS-CoV-2 was investigated next. Since the full ORF1ab could not be produced due to its extended length (>21,000 bp, (1)), we a representative antigen, non-structural protein 1 (NSP1) was cloned and expressed. Bioinformatic predictions have revealed a putative role for NSP1 in suppressing the antiviral host response (8).

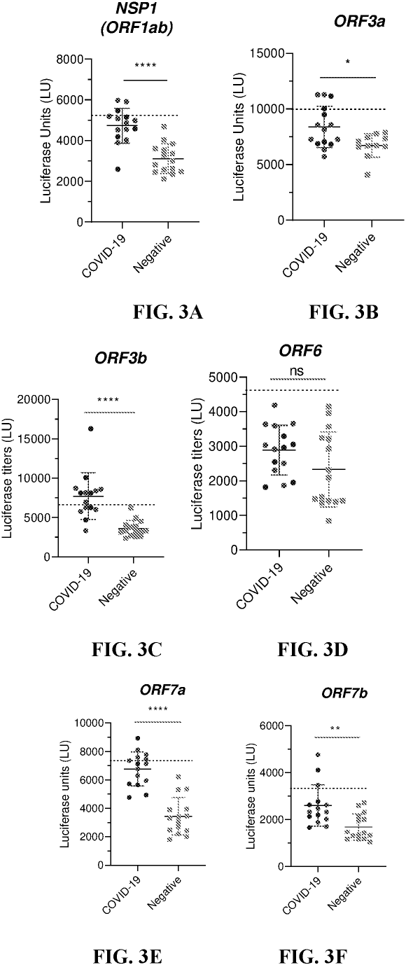

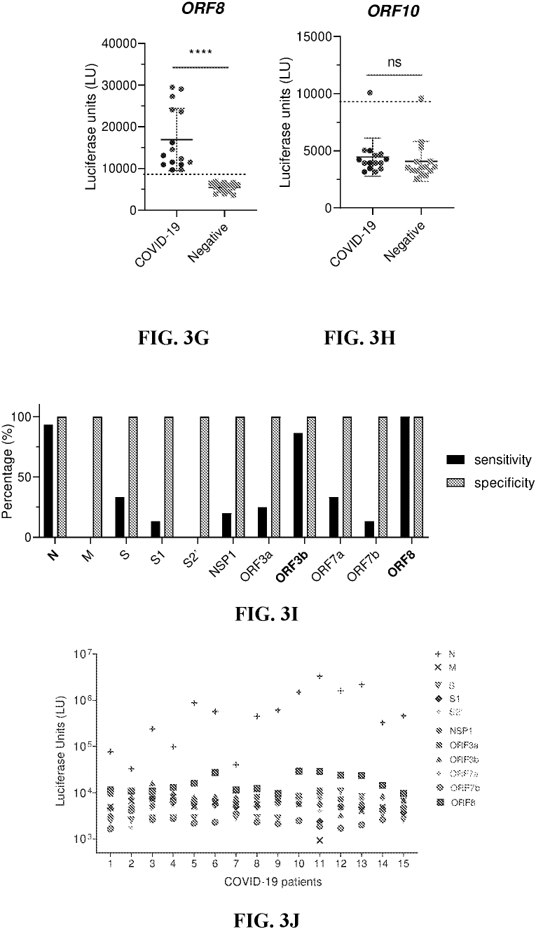

LIPS was used to detect antibodies specific to NSP1, ORF3a, ORF3b, ORF6, ORF7a, ORF7b, ORF8 and ORF10 (FIGS. 3A-I)). COVID-19 patients had significantly higher NSP1-antibody levels compared to healthy controls (mean of 5301+/-854.7 LU versus 3683+/-726.4 LU, p<0.0001, FIG. 3A). Furthermore, significantly higher antibody levels in COVID-19 patients were detected towards ORF3a, ORF3b, ORF7a, ORF7b and ORF8 (p=0.0302, p<0.0001, p<0.0001, p=0.0019, p<0.0001, FIGS. 3B, C, D, F, G). The largest difference between COVID-19 and healthy controls in mean antibody signals among the ORFs was observed for ORF3b (7712+/-2947 LU versus 3599+/-1029 LU) and ORF8 (16933+/-7489 LU versus 5440+/-1096 LU) (FIGS. 3C and G). Meanwhile there was no significant difference between COVID-19 patients and healthy controls for ORF6 and ORF10 antigens (FIG. 3D and H).

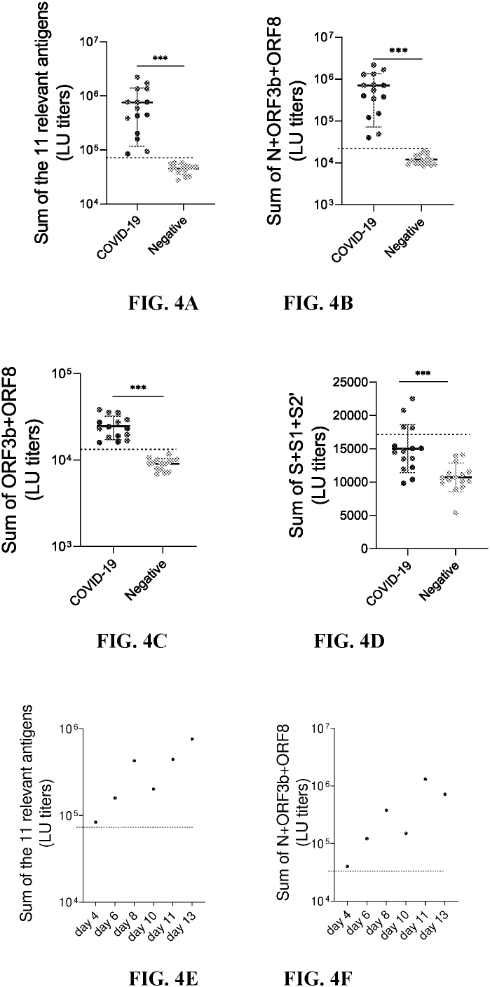

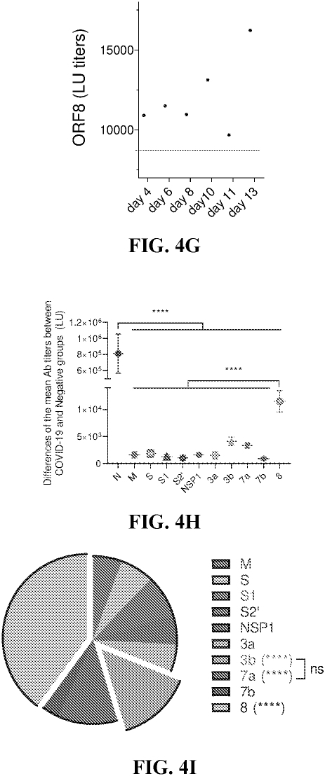

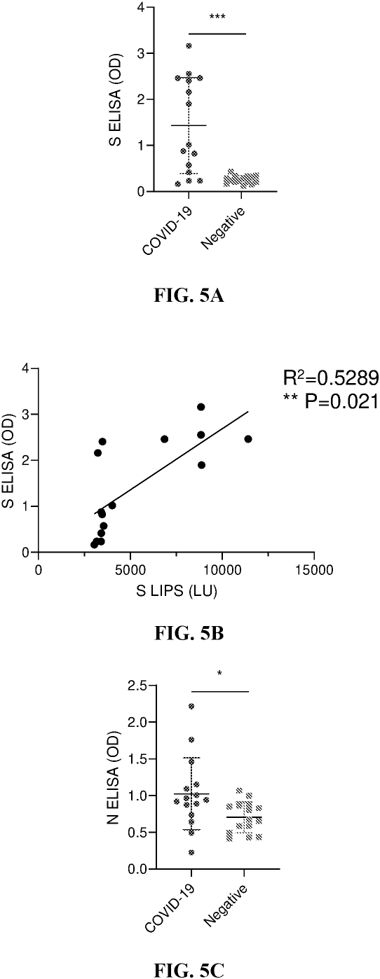

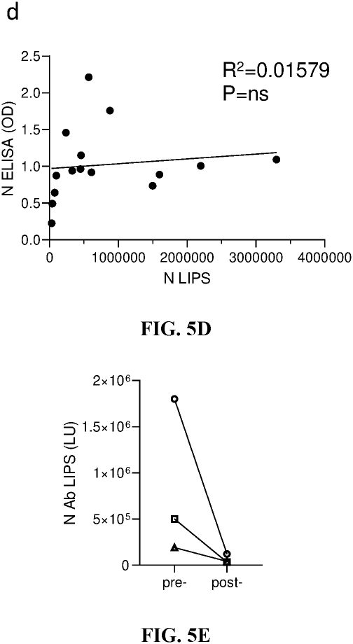

Globally, among the antibody responses tested in this LIPS assay, we detected significant levels of antibodies specific to 11 antigens in the COVID-19 population (Table 1) at all time-points of infection from day 4 to day 22 (see below in FIGS. 4A-L for early time-point detection). A significant production of antibodies to 4 of the SARS-CoV-2 antigens tested was not detected: E, S2, ORF6 and ORF10 proteins. Therefore, the sensitivity and specificity of different SARS-CoV-2 antigens (FIG. 3I), showed N, ORF3b and ORF8 outperformed other viral antigens.

Comparison of the global SARS-CoV-2 antibody responses from 15 COVID-19 patients reveals that anti-N antibodies dominate the humoral response detected by LIPS (FIG. 3J), whilst other antigens make lower and similar contributions to the magnitude anti-SARS-Cov-2 antibody responses (FIG. 4HI).

### Absence of Sex- and Age- Effect in Our Cohort

Epidemiological data from many countries suggest that men develop more severe COVID-19 related symptoms than women (21). The effect of gender in the studied cohort of COVID-19 patients was therefore evaluated for antibody responses and found no difference across all antigenic targets tested (p=0.6289, FIG. 6A). Similarly, the SARS-CoV-2 antibody response was comparable between patients aged below and above 60 years old (p=0.9363, FIG. 6B).

### Combined Antigen Test Panels as a Potential Diagnostic Tool for COVID-19

Significantly higher levels of 11 of the 15 antigens tested were found in the COVID-19 populations. Next, their ability to correctly identify COVID-19 patients was determined. Therefore, a cut-off value of LIPS antibody signals, based on the mean plus three standard deviations of the healthy negative control group (22-24), was calculated for each of these 11 antigens, along with the sensitivity and specificity of each test (Table 5, and FIGS. 3I, 4A-G).

TABLE 5

Cut-off value and sensitivity for the 11 relevant SARS-CoV-2 antigens by LIPS, and the sum of the 11 relevant tests, the sum of N+ORF3b+ORF8 tests, the sum of ORF3b+ORF8 tests, and the sum of S+S1+S2′ tests.

| Antigen | Cut-off value | sensitivi ty | specificity | Positive predictive value (PPV) | Negative predictive value (NPV) |
|---|---|---|---|---|---|
| N | 46133 | 93.3% | 100% | 100% | 93.75% |
| M | 8684 | 0% | 100% | 0% | 50% |
| S | 4333 | 33.3% | 100% | 100% | 60% |
| S1 | 7189 | 13.3% | 100% | 100% | 53.57% |
| S2′ | 7635 | 0% | 100% | 0% | 50% |
| NSP1 | 5862 | 20% | 100% | 100% | 55.56% |
| ORF3a | 9925 | 25% | 100% | 100% | 55.56% |
| ORF3b | 6686 | 86.6% | 100% | 100% | 88.24% |
| ORF7a | 7394 | 33.3% | 100% | 100% | 60% |
| ORF7b | 3355 | 13.3% | 100% | 100% | 53.57% |
| ORF8 | 8728 | 100% | 100% | 100% | 100% |
| Sum of the 11 relevant tests | 73323 | 100% | 100% | 100% | 100% |
| Sum of N+ORF3b+ORF8 | 22101 | 100% | 100% | 100% | 100% |
| Sum of ORF3b+ORF8 | 13280 | 100% | 100% | 100% | 100% |
| Sum of S+S1+S2′ | 17134 | 26.67 | 100% | 100% | 57.69% |

The cut-off value of all 11 tests showed a high specificity of the SARS-CoV-2 LIPS assays with no samples from the negative control group being above the cut-off. On the other hand, 8 antigens showed a low sensitivity (below 75% for M, S, S1, S2′, NSP1, ORF3a, ORF7a, ORF7b, FIG. 4A), hence being insufficient to correctly identify all the COVID-19 patients with high rates of false negatives (Table 5), limiting their use for LIPS diagnostics. On the other hand, the N, ORF3b and ORF8 antigens showed good sensitivity levels of 93.3%, 86.6% and 100% respectively (Table 5). Of note, ORF8 is the only antigen of the 11 tests to identify correctly all the COVID-19 patients including at day 4 after onset of illness (FIG. 3I).

As many LIPS tests showed low sensitivity, an approach based on the combination of LIPS antibody LU signals for the 11 separate SARS-CoV-2 antigen tests was then used to efficiently detect SARS-CoV-2 exposure in this population (22-24). This approach of combining the LU values from the 11 LIPS tests increased the sensitivity to 100% for the diagnosis of COVID-19 infections during acute infection (FIG. 4A, blue dots for <14 days). Further, combination of only 3 LIPS tests, those for anti-N, ORF3b and ORF8 antibodies, also has a 100% sensitivity and specificity (FIG. 4B and Table 5). As ORF3b and ORF8 show the lowest homology to previous SARS-CoV among all the viral proteins (8), the combination of responses towards ORF3b and ORF8 were also studied separately (FIG. 4C). The same trend as above was observed, with all COVID-19 patients having a combined score above the cut-off and all negative controls having a combined score below, and all early time-points being correctly detected (FIGS. 4E-G). By combining only the 3 relevant Spike protein LIPS tests (S, S1, S2′), the sensitivity of the LIPS test drastically decreased to 26.6% as only 4 of the COVID-19 patients had a total combined LU above the cut-off (FIG. 4D). Interestingly all samples from early time-points (day 4 to day 13) had LU signals under the cut-off value for S+S1+S2′ (blue dots, FIG. 4D). The magnitude of the 11 relevant antigens (FIG. 4E) and N+ORF3b+ORF8 (FIG. 4F) responses significantly increase the detection of patients with early time-points samples. Therefore, the combinational use of ORF3b and ORF8 tests alone could be sufficient to detect COVID-19 exposed subjects at any time-point of infection.

Because endemic HCoVs are ubiquitous, the negative control plasma samples are likely to have antibodies to a range of HCoVs. Sequence homology with other HCoV could result in the detection of cross-reactive antibodies by LIPS and reduce specificity of serological assays. Structural proteins of SARS-CoV-2 with other HCoV structural proteins only share 18 to 40% homology (Table 4), making potential cross-reactivity of existing antibodies unlikely and undetermined. Whilst SARS-Cov-2 and SARS-CoV, N and S share 94% and 76% conservation (Table 4), previous reports showed the lowest homology for ORF3b and ORF8 at 32% and 40% amino acid homology respectively (8), making them the most unique genes to SARS-CoV2.

To investigate specificity of the antibody responses to the panel of antigens, the mean LU levels of healthy negative controls was subtracted from the mean LU of COVID-19 infected patients and compared this difference across each antigen (FIG. 4H). This difference was found to be significantly higher for the N-specific antibody response compared to all the other relevant antibody responses (M, S, S1, S2′, NSP1, ORF3a, ORF3b, ORF7a, ORF7b, and ORF8) (p<0.0001, FIG. 4I), highlighting a possible dominance and specificity of N across the SARS-CoV-2 humoral immune responses. Besides N, the ORF8 and ORF3b also appear to be important antigenic targets (FIGS. 4H, I). Statistical analysis (ANOVA) revealed that ORF8 was significantly increased compared to all other antigens (p<0.0001 versus 10 remaining antigens: M, S, S1, S2′, NSP1, ORF3a, 3b, 7a, and 7b, FIG. 4HI). Whilst results for ORF3b were significant against the remaining 8 antigens (M, S, S1, S2′, NSP1, ORF3a, 3b, and 7b), excluding ORF7a (FIG. 4HI). Therefore, ORF3b and ORF8 are newly identified as specific and unique antigenic targets.

### Discussion

Using LIPS technology with crude lysates from transfected cells, we have screened all the structural proteins along with all the ORFs of the SARS-CoV-2 virus (NSP1 only for ORF 1ab) to identify new and unique antigenic targets of the humoral immune response of COVID-19 patients.

Among the 15 proteins tested for antibody specificity, 11 antigens showed significantly higher responses in the COVID-19 patients compared to healthy pre-pandemic controls. For the Spike subunits, only antibodies to S1 and S2′ were elevated in COVID-19 patients by the LIPS test conducted herein, and S2 responses were not significantly different from the control group. The trimer S conformation and maturation of viral particles by the cleavage of S2 during virus endocytosis to form the S2′ fusion peptide may result in a difference in their antigenicity. Patients with higher MN titer had correspondingly higher levels of full-Spike LIPS and ELISA results, establishing consistency among assays for the full-S protein. N antibody responses detected were also elevated in patients, but the correlation between LIPS and ELISA N antibody assays was lower.

Next all the available ORFs of the virus ORF1ab were cloned (as NSP1 only), ORF3a, ORF3b, ORF6, ORF7a, ORF7b, ORF8 and ORF10, to acquire more extensive information of the immunogenic targets of the virus. The studies showed that 6 of these 8 ORFs induced a humoral immune response in the patients (NSP1 (ORF1ab), ORF3a, ORF3b, ORF7a, ORF7b and ORF8). Furthermore, by calculating a cut-off value based on the mean of the negative population + 3 standard deviations, it was ascertained that only 3 out of these 11 antigens were useful in diagnostic tests with high performance: N, ORF3b, and ORF8. By using the combined results of several antibody signals and calculating a cut-off value for these responses, it was found that the sum of the 11 relevant antibody tests was highly sensitive and specific. Moreover, combining only the 3 of the most informative and sensitive antigens, N, ORF3b and ORF8, a sensitivity and specificity of 100% was achieved, correctly identifying all the COVID-19 patients versus negative controls.

The Spike protein is responsible for virus entry into host cells and is the main antigen that elicits neutralizing antibodies (10). Whilst significant differences were detected in the magnitude of responses by LIPS between patients and controls for S, S1 and S2′, these antigens did not show high sensitivity levels, especially for sera collected early post disease onset. The S protein also elicits non-neutralizing antibodies targeted to conserved epitopes (11), and among the presentably tested cohort an absence of in vitro neutralization has been observed for some patients, especially at early time-points.

The combination of multiple antigens by LIPS beyond the Spike could be the basis for supplementary serological tests useful to determine SARS-CoV-2 exposure to overcome false negative results. The present studies showed that the single test detection of N, S or ORF3b antibodies at early time-points (day 4 to day 14) results in a high proportion of false negative results, whilst the minimal combination of N+ORF3b+ORF8 LIPS tests is highly sensitive and specific. Importantly, ORF3b and ORF8 are the least identical proteins to SARS-CoV (8), and they do not exist in other strains of human coronaviruses. However very little is known about their function and expression. Previous reports found the ORF3b of SARS-CoV plays an important role in the interaction with the innate immune system through inhibition of type 1 Interferon synthesis (33). In SARS-CoV, ORF8 has been shown to accumulate in the Endoplasmic Reticulum and mediate cell death by autophagy (32). In SARS-CoV-2, the functions of these ORFs have yet to be determined. Importantly, ORF8 can only be found in human and bat SARS-like CoV (35), and the present studies observed very low background detection in negative control plasma resulting in highly specific results. Whilst ORF8-deletion SARS-CoV-2 viruses had reduced replicative fitness, this issue may undermine the utility of ORF8 alone in serological testing. Most commercially available or published serological tests use only the S antigen, with a few using both the S and N antigens (11, 36, 37). Extensive testing of the virus antigens show here that additional targets are important for early detection of antibody responses and identification of COVID-19 patients. The high orders of magnitude and range of antibody quantity measured by our SARS-CoV-2 LIPS assay is an advantage compared to ELISA Optical Density measurements. These advantages may help with the rapid screening of recovered COVID-19 patients for elevated antibodies to donate serum for the treatment of other patients by passive transfer (38).

Only IgG is bound to the protein A/G used in the LIPS assays, therefore the use of protein L that binds the light chain of all immunoglobulins can be used to detect IgM early responses. The E, S2, ORF6 and ORF10 antibodies did not show elevated levels in COVID-19 patients in our assay, consistent with the findings of Wang H. et al. by microarray (29).

In conclusion, the studies showed that the combined use of N+ORF3b+ORF8 provides a sensitive and specific method for the detection of all COVID-19 patients in our cohort even at early time-points, whilst the Spike protein does not.

### References

1. Wu F, Zhao S, Yu B, Chen YM, Wang W, Song ZG, et al. A new coronavirus associated with human respiratory disease in China. Nature. 2020;579(7798):265-9.
2. Poon LLM, Peiris M. Emergence of a novel human coronavirus threatening human health. Nat Med. 2020;26(3):317-9.
3. Amanat F, Krammer F. SARS-CoV-2 Vaccines: Status Report. Immunity. 2020.
4. Liu Y, Yan LM, Wan L, Xiang TX, Le A, Liu JM, et al. Viral dynamics in mild and severe cases of COVID-19. Lancet Infect Dis. 2020.
5. World Health Organization. Coronavirus disease (COVID-2019) situation reports 2020 [Available from: https://www.who.int/emergencies/diseases/novel-coronavirus-2019/situation-repo rts.
6. Zhang C, Zheng W, Huang X, Bell EW, Zhou X, Zhang Y. Protein Structure and Sequence Reanalysis of 2019-nCoV Genome Refutes Snakes as Its Intermediate Host and the Unique Similarity between Its Spike Protein Insertions and HIV-1. J Proteome Res. 2020; 19(4): 1351-60.
7. Thevarajan I, Nguyen THO, Koutsakos M, Druce J, Caly L, van de Sandt CE, et al. Breadth of concomitant immune responses prior to patient recovery: a case report of non-severe COVID-19. Nat Med. 2020;26(4):453-5.
8. Chan JF, Kok KH, Zhu Z, Chu H, To KK, Yuan S, et al. Emerg Microbes Infect. 2020;9(1):221-36.
9. Walls AC, Park YJ, Tortorici MA, Wall A, McGuire AT, Veesler D. Structure, Function, and Antigenicity of the SARS-CoV-2 Spike Glycoprotein. Cell. 2020.
10. Paules CI, Marston HD, Fauci AS. Coronavirus Infections-More Than Just the Common Cold. JAMA. 2020.
11. Huibin Lv NCW, Owen Tak-Yin Tsang, Meng Yuan, Ranawaka A. P. M. Perera, Wai Shing Leung, Ray T. Y. So, Jacky Man Chun Chan, Garrick K. Yip, Thomas Shiu Hong Chik, Yiquan Wang, Chris Yau Chung Choi, Yihan Lin, Wilson W. Ng, Jincun Zhao, Leo L. M. Poon, J. S. Malik Peiris, Ian A. Wilson, Chris K. P. Mok. Cross-reactive antibody response between SARS-CoV-2 and SARS-CoV infections. bioRxiv. 2020.
12. Thevarajan I, Nguyen THO, Koutsakos M, Druce J, Caly L, van de Sandt CE, et al. Breadth of concomitant immune responses prior to patient recovery: a case report of non-severe COVID-19. Nature Medicine. 2020.
13. Wu F, Wang A, Liu M, Wang Q, Chen J, Xia S, et al. Neutralizing antibody responses to SARS-CoV-2 in a COVID-19 recovered patient cohort and their implications. medRxiv. 2020:2020.03.30.20047365.
14. Choe PG, Perera R, Park WB, Song KH, Bang JH, Kim ES, et al. MERS-CoV Antibody Responses 1 Year after Symptom Onset, South Korea, 2015. Emerg Infect Dis. 2017;23(7):1079-84.
15. Hemida MG, Chu DK, Poon LL, Perera RA, Alhammadi MA, Ng HY, et al. MERS coronavirus in dromedary camel herd, Saudi Arabia. Emerg Infect Dis. 2014;20(7):1231-4.
16. Channappanavar R, Zhao J, Perlman S. T cell-mediated immune response to respiratory coronaviruses. Immunol Res. 2014;59(1-3): 118-28.
17. Burbelo PD, Goldman R, Mattson TL. A simplified immunoprecipitation method for quantitatively measuring antibody responses in clinical sera samples by using mammalian-produced Renilla luciferase-antigen fusion proteins. BMC Biotechnol. 2005;5:22.
18. Robertson AH, Mahic M, Savic M, Tunheim G, Hungnes O, Trogstad L, et al. Detection of anti-NS1 antibodies after pandemic influenza exposure: Evaluation of a serological method for distinguishing H1N1pdm09 infected from vaccinated cases. Influenza Other Respir Viruses. 2020.
19. Uehara A, Tan CW, Mani S, Chua KB, Leo YS, Anderson DE, et al. Serological evidence of human infection by bat orthoreovirus in Singapore. J Med Virol. 2019;91(4):707-10.
20. Burbelo PD, Ching KH, Klimavicz CM, Iadarola MJ. Antibody profiling by Luciferase Immunoprecipitation Systems (LIPS). J Vis Exp. 2009(32).
21. Conti P, Younes A. Coronavirus COV-19/SARS-CoV-2 affects women less than men: clinical response to viral infection. J Biol Regul Homeost Agents. 2020;34(2).
22. Burbelo PD, Keller J, Wagner J, Klimavicz JS, Bayat A, Rhodes CS, et al. Serological diagnosis of pulmonary Mycobacterium tuberculosis infection by LIPS using a multiple antigen mixture. BMC Microbiol. 2015;15:205.
23. Frey A, Di Canzio J, Zurakowski D. A statistically defined endpoint titer determination method for immunoassays. J Immunol Methods. 1998;221(1-2):35-41.
24. Burbelo PD, Chaturvedi A, Notkins AL, Gunti S. Luciferase-Based Detection of Antibodies for the Diagnosis of HPV-Associated Head and Neck Squamous Cell Carcinoma. Diagnostics (Basel). 2019;9(3).
25. Nie J, Li Q, Wu J, Zhao C, Hao H, Liu H, et al. Establishment and validation of a pseudovirus neutralization assay for SARS-CoV-2. Emerg Microbes Infect. 2020;9(1):680-6.
26. Fatima Amanat DS, Shirin Strohmeier, Thi Nguyen, Veronika Chromikova, Meagan McMahon, Kaijun Jiang, Guha Asthagiri-Arunkumar, Denise Jurczyszak, Jose Polanco, Maria Bermudez-Gonzalez, Giulio Kleiner, Teresa Aydillo, Lisa Miorin, Daniel Fierer, Luz Amarilis Lugo, Erna Milunka Kojic, Jonathan Stoever, Sean T.H. Liu, Charlotte Cunningham-Rundles, Philip L. Felgner, Daniel Caplivski, Adolfo Garcia-Sastre, Allen Cheng, Katherine Kedzierska, Olli Vapalahti, Jussi Hepojoki, Viviana Simon, Florian Krammer, Thomas Moran. A serological assay to detect SARS-CoV-2 seroconversion in humans. medRxiv. 2020.
27. Pan Y, Li X, Yang G, Fan J, Tang Y, Zhao J, et al. Serological immunochromatographic approach in diagnosis with SARS-CoV-2 infected COVID-19 patients. medRxiv. 2020.
28. Christine Dahlke JH, Robin Kobbe, Rene Santer, Till Koch, Anahita Fathi, My L. Ly, Stefan Schmiedel, Peter H. Seeberger, ID-UKE COVID-19 study group, Marylyn M. Addo, Felix F. Loeffler. Distinct early IgA profile may determine severity of COVID-19 symptoms: an immunological case series. medRxiv. 2020.
29. Hongye Wang XH, Xian Wu, Te Liang, Xiaomei Zhang, Dan Wang, Fei Teng, Jiayu Dai, Hu Duan, Shubin Guo, Yongzhe Li, Xiaobo Yu. SARS-CoV-2 proteome microarray for mapping COVID-19 antibody interactions at amino acid resolution. bioRxiv. 2020.
30. Bujang MA, Adnan TH. Requirements for Minimum Sample Size for Sensitivity and Specificity Analysis. J Clin Diagn Res. 2016;10(10):YE01-YE6.
31. Yuan M, Wu NC, Zhu X, Lee CD, So RTY, Lv H, et al. A highly conserved cryptic epitope in the receptor-binding domains of SARS-CoV-2 and SARS-CoV Science. 2020.
32. McBride R, Fielding BC. The role of severe acute respiratory syndrome (SARS)-coronavirus accessory proteins in virus pathogenesis. Viruses. 2012;4(11):2902-23.
33. Shi CS, Nabar NR, Huang NN, Kehrl JH. SARS-Coronavirus Open Reading Frame-8b triggers intracellular stress pathways and activates NLRP3 inflammasomes. Cell Death Discov. 2019;5:101.
34. Yvonne CF Su DEA, Barnaby E Young, Feng Zhu, Martin Linster, Shirin Kalimuddin, Jenny GH Low, Zhuang Yan, Jayanthi Jayakumar, Louisa Sun, Gabriel Z Yan, Ian H Mendenhall, Yee-Sin Leo, David Chien Lye, Lin-Fa Wang, Gavin JD Smith. Discovery of a 382-nt deletion during the early evolution of SARS-CoV-2. bioRxiv. 2020.
35. Cui J, Li F, Shi ZL. Origin and evolution of pathogenic coronaviruses. Nat Rev Microbiol. 2019;17(3):181-92.
36. Zhang W, Du RH, Li B, Zheng XS, Yang XL, Hu B, et al. Molecular and serological investigation of 2019-nCoV infected patients: implication of multiple shedding routes. Emerg Microbes Infect. 2020;9(1):386-9.
37. Nisreen M.A. Okba MAM, Wentao Li, Chunyan Wang, Corine H. GeurtsvanKessel, Victor M. Corman, Mart M. Lamers, Reina S. Sikkema, Erwin de Bruin, Felicity D. Chandler, Yazdan Yazdanpanah, Quentin Le Hingrat, Diane Descamps, Nadhira Houhou-Fidouh, Chantal B. E. M. Reusken, Berend-Jan Bosch, Christian Drosten, Marion P.G. Koopmans, Bart L. Haagmans. SARS-CoV-2 specific antibody responses in COVID-19 patients. bioRxiv. 2020.
38. Casadevall A, Pirofski LA. The convalescent sera option for containing COVID-19. J Clin Invest. 2020;130(4):1545-8.
39. Fu Y, Cheng Y, Wu Y. Understanding SARS-CoV-2-Mediated Inflammatory Responses: From Mechanisms to Potential Therapeutic Tools. Virol Sin. 2020.

### Example 2

In Example 1, we used LIPS to initially assess the antibody responses to a panel of 15 SARS-CoV-2 antigens representing the structural and non-structural viral proteins in 15 COVID-19 patients and 15 pre-pandemic negative controls. Antibodies to the 4 structural proteins (S, N, M and E), 3 S subunits (S1, S2, S2′), the 7 available ORFs (ORF3a, 3b, 6, 7a, 7b, 8 and 10), and 1 relevant NSP within ORF1a/b (NSP1) were tested. A cut-off calculated as mean of the results on the negative plasma samples plus 3 standard deviations allowed the selection of sensitive and specific tests. In this second study, Example 2, we then further validated our selected assays on a larger panel of sera totaling 84 COVID-19 patient plasma from early (n=51, <14 days post symptom onset) and late time-points (n=33, >14 days post symptom onset) and 176 negative controls to assess assay performances of relevant N, ORF3b and ORF8 antigenic targets.

Finally, sequential samples from patients were tested to determine the kinetics of the antibody response.

### Methods

### Patients and Samples Collection

Our study enrolled a total of 84 adult patients based on recruitment of available patients with RT-PCR confirmed COVID-19 infection in Hong Kong, and used 84 COVID-19 patient plasma samples including 14 subjects with 2 to 4 sampling time points, and 51 early time-points samples (≤ day 14). Sample day was defined as day post-symptom onset. The COVID-19 patient study was approved by the institutional review board of the Hong Kong West Cluster of the Hospital Authority of Hong Kong (approval number: UW20-169), and all of patients provided informed consent.

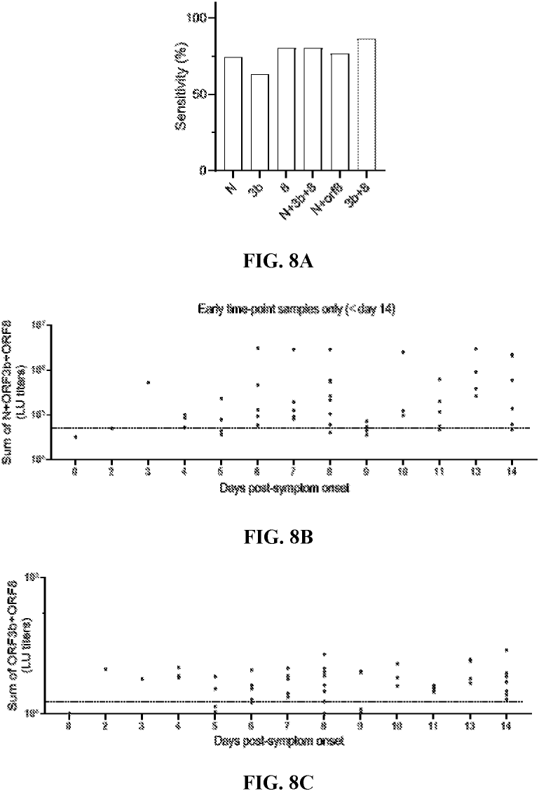

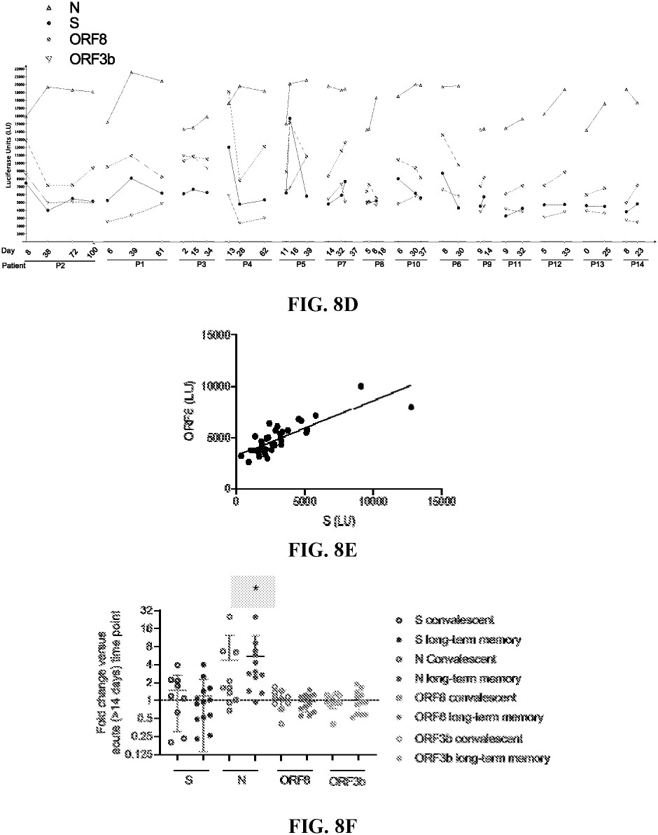

To increase sample size and validate findings on a second set of samples that were tested (FIGS. 8-10), a larger panel of 176 additional negative control plasma from healthy subjects (minimum age of 16 years-old, maximum age of 65 years-old) were collected before the COVID-19 pandemic. This negative control plasma was from Hong Kong blood donors collected from June to August 2017 (prior to the emergence of COVID-19), which was recently used as a control cohort for neutralisation assays. The collection of negative control blood donors was approved by the Institutional Review Board of The Hong Kong University and the Hong Kong Island West Cluster of Hospitals (IRB reference number UW16-254). Plasma samples were collected from heparinized blood, and heat-inactivated prior to experimental use at 56° C., 30 mins.

### SARS-CoV-2 Gene Cloning

Based on previous studies describing the structure of the SARS-CoV-2 genome, an extensive panel of 12 proteins (S, E, M, N, NSP1, ORF3a, 3b, 6, 7a, 7b, 8, 10) was chosen for antibody testing by LIPS (Table 1). Primers for the amplification of SARS-CoV-2 proteins were designed (table2). A RT-PCR was performed using RNA extracted from SARS-CoV-2 virus strain BetaCoV/Hong Kong/VM20001061/2020 grown in Vero E6 cells to amplify target genes corresponding to Structural and Non-structural proteins of the virus (Table 1) using Platinum SuperScriptIII One Step RT-PCR system. The bands were then extracted using Qiagen gel extraction kit (Qiagen, Germany) and digested with BamHI and NotI or KpnI-HF and XhoI (New England Biolabs, USA). Extracted products were ligated using T4 DNA ligase (New England Biolabs) into the pREN2 plasmid (from Peter Burbelo NDICR, NIH). Plasmids were transformed using DH10B competent cells and purified using Pure Yield Plasmid mid-prep system (Promega). Constructs were confirmed by Sanger Sequencing (3730x1 DNA Analyzer Applied Biosystems).

### SARS-CoV-2 (Ruc)-Antigen Expression

Constructs with pREN2-Renilla luciferase plasmid containing the SARS-CoV-2 antigen of interest were transfected into Cos1 cells using Fugene 6 (Promega) as per manufacturer’s instructions. Cells were harvested 48 hours later, lysed and sonicated, and (Ruc)-antigen yields were measured using a Luminometer plate reader (PerkinElmer) according to the protocol of Burbelo et al. Each Ruc-fusion antigen is tested for its LU yield after production in Cos1 cells. The LU was then standardized to 10⁷ LU per antigen in each well before each LIPS assay, therefore different yields during transfection for recovery of luciferase tagged proteins is controlled for the assay and plasma samples run against the same amount of each antigen.

### Measurement of Antibody Responses Using LIPS

The LIPS assays were performed following the protocol of Burbelo et al., with the following modifications.Briefly, (Ruc)-antigen (at an equal concentration for each antigen at 10⁷ per well) and plasma (heat inactivated and diluted 1:100) were incubated for 2 hours with shaking at 800 rpm. Ultralink protein A/G beads were added to the (Ruc)-antigen and serum mixture in a 96-deep-well polypropylene microtiter plate and incubated for 2 hours with shaking at 800 rpm. The entire volume was then transferred into HTS plates and washed as previously described. The plate was read using QUANTI-Luc Gold substrate (Invivogen) as per manufacturer’s instructions on a Wallac MicroBeta JET luminometer 1450 LSC & Luminescence counter and its software for analysis (PerkinElmer). Experimental controls include no plasma blank wells with (Ruc)-antigens and negative control serum from age matched non-infected patient plasma collected prior to the COVID-19 pandemic. The background corresponds to the LU signal from each Ruc-fusion antigen with protein A/G and substrate with no plasma.

### Enzyme-Linked Immunosorbent Assay

ELISA assays were performed with the available SARS-CoV-2 proteins Spike (S1+S2) and Nucleoprotein (N) proteins as well as HCoV OC43, NL63 and 229E Spike (SinoBiological). Briefly, recombinant S and N proteins were coated on 96-well flat-bottom immunosorbent plates (Nunc Immuno MaxiSorp, Roskilde, Denmark) at a concentration of 500 ng/ml, in 100/µl coating buffer (PBS with 53% Na₂CO₃ and 42% NaHCO₃, pH 9.6) at 4° C. overnight. An additional plate coated with a non-specific protein (blocking buffer, PBS with 5% FBS) was used to measure the background binding of each plasma sample. Following FBS blocking and thorough washing, diluted plasma samples (1:100) were bound for 2 hours, further washed and then detected by an anti-human IgG secondary antibody labelled with HRP (Invitrogen, Carlsbad, CA, USA) at 1:5,000 dilution.

### N Adsorption

Plasma (n=3) were incubated on 5 µg/ml N protein coated 96 round-well plate with shaking at 800 rpm at 37° C. for 2 hours twice. N LIPS was then performed as described above.

### Microneutralization Assay

See Example 1

### Multiple Alignments of Coronaviruses

See Example 1

### ORF3b-ORF8 Cluster of Points

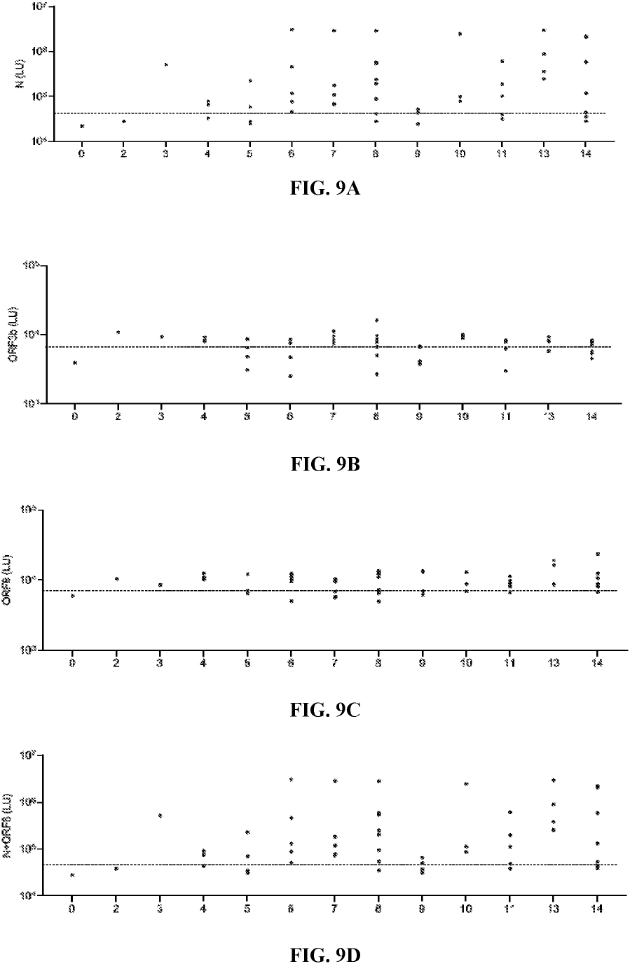

The ORF3b and ORF8 full dataset has been treated through the free software ConTeXt, with LuaMetaTeXengine (version 2020.05.18) developed by Hans Hagen (http://www.pragma-ade.nl), which uses TeX, Metapost and Lua to obtain the 2D cloud shown in FIG. 9G. The red line (equation: 0.2185ORF3b + 0.5927 ORF8= 4643.1972) allows the most accurate discrimination between negative controls (grey dots) and positive patients (red dots) populations.

### Statistics and Reproducibility

GraphPad Prism version 8 software (San Diego, CA) was used for statistical analysis. Antibody levels are presented as the geometric mean +/- standard deviation (stdev). For the calculation of sensitivity (% of true positives, absence of true negatives in the positive population) and specificity (% of true negatives, absence of false positives in the negative population), cut-off limits for each antigen were derived from the mean value plus three standard deviations of the controls. The positive predictive value (PPV) (n of true positives/ (true + false positives)), and negative predicted value (n of true negatives/(true + false negatives)) were also determined (Table 5). Non-parametric two-sided Mann-Whitney U tests were used to compare the antibody levels between COVID-19 and negative groups. In FIG. 4H an ordinary one-way ANOVA with Tukey’s multiple comparison test was performed. Due to the differences in scale between N and all other 10 antigens, a second ordinary one-way ANOVA with Tukey’s multiple comparison test was performed excluding N to determine the dominance amongst the remaining 10 antigens. All experiments were repeated twice independently. Further information on research design is available in the Nature Research Reporting Summary linked to this article.

### Results

### Validation of LIPS in a Second Larger Cohort and Two-Dimensional Plane For Diagnosis by ORF3b and ORF8

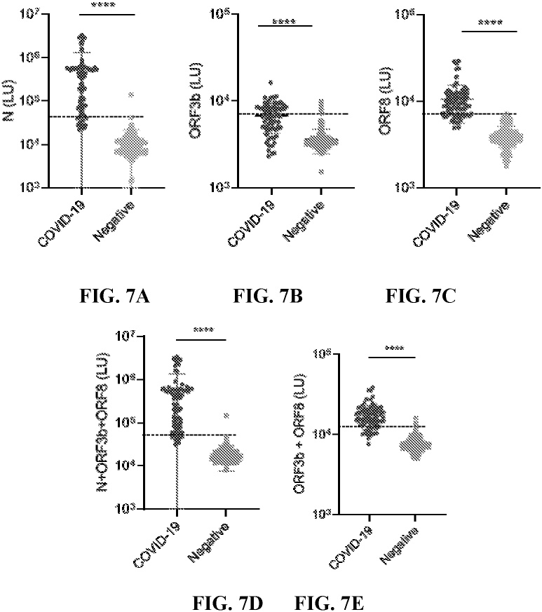

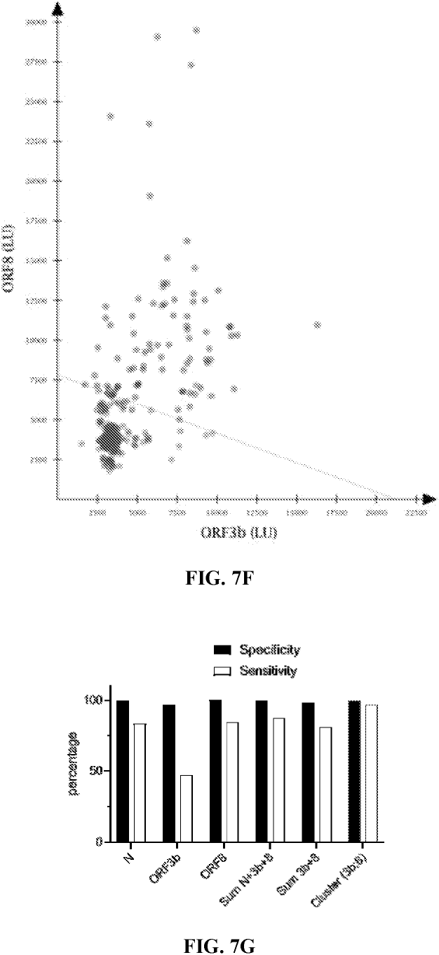

To validate these findings, we further increased our sample size to 84 COVID-19 plasmas and 176 negatives for N (FIG. 7A), ORF3b (FIG. 7B) and ORF8 (FIG. 7C) antibody responses. We observed high specificity (>95%) for all three tests as above (FIG. 7D), and N (83.3% sensitivity) and ORF8 (84.5% sensitivity) showed the highest sensitivity (FIG. 7C). Whilst the sum of N+ORF3b+8 increased the sensitivity to 87.5% (FIG. 7D). Representation of the ORF3b and ORF8 antibody values in a two-dimensional plane (x, y) allowed a clearer visualization and definition of the COVID-19 (red) and negative cohorts (grey) (FIG. 7F). Indeed, by introducing an optimal discriminant line (the line parallel to the vector (0.2185; 0.5927) in the plane), we could decipher clearly positive and negative populations. Only one patient out of 176 negative control group was an outlier, and all other negative patients are clearly identified as negative with a 99.5% specificity. Meanwhile, the sensitivity was also increased to 96.5% for the cluster analysis versus 81% for the sum of ORF3b+ORF8 (FIG. 7G). This geometric interpretation allows a highly accurate diagnosis of COVID-19 and emphasizes the diagnostic relevance of these two antibody responses, ORF3b and ORF8 to be considered together.

### Early Diagnostic Use of ORF3b+ORF8 Specific Antibodies by LIPS

One of the main challenges of the serologic diagnosis of SARS-CoV-2 is identification of infection at early time-points. The assessment of the N, ORF3b and ORF8 tests from day 0 to day 14 samples revealed that 25.5%, 37.3% and 19.6% of the samples were mis-identified (below the cut-off, FIGS. 8A-C, 9). However, using the ORF3b+ORF8 sum improved the diagnosis of these early time-point samples with a sensitivity of 86.4%, allowing the correct detection of 44 out of 51 samples within 14 days of symptom onset (FIG. 8C).

### Characterisation of Longitudinal Antibody Responses of S, N, ORF3b and ORF8

Longitudinal studies enable the assessment of kinetics of antibody responses, especially antibody waning. Longitudinal and paired samples from two to four time-points were collected from 14 patients for longitudinal measurement of S, N, ORF3b and ORF8 antibody responses. The antibody responses to N, S, ORF3b and ORF8 are well maintained over time (even up to day 100) (FIG. 8D). Of note, amongst the 4 antibodies tested, ORF8 and S antibodies followed a very similar trend in all patients (FIG. 8D, blue (S) and green lines (ORF8)). Indeed, the kinetics of the ORF8 and S specific antibody responses by LIPS significantly correlated (R²=0.66902, p<0.0001) (FIG. 8E), whilst the other antibody responses did not. Previously, ORF8 in SARS-CoV infected cells was reported to have a strong association with the Spike protein whilst also inhibiting expression of Envelope protein, which may account for the serological trend observed here in SARS-CoV-2 infection.

The fold changes (FIG. 8F) of antibody levels from acute (<14 days post symptom onset) to convalescent (day 14-30) and long-term memory (day >31) responses were determined from longitudinal samples. The N response is significantly increased at long-term memory (p=0.0359), whilst the S, ORF3b and ORF8 all show a similar trend of maintained responses with a fold change close to 1 (FIG. 8F). Of note, ORF8 and ORF3b responses are the most stable across patients (with the narrowest standard deviation) over time, making them ideal marker of acute and past infection.

### Lack of Cross Reactivity of Existing HCoV Responses for N, ORF3b, and ORF8 by LIPS

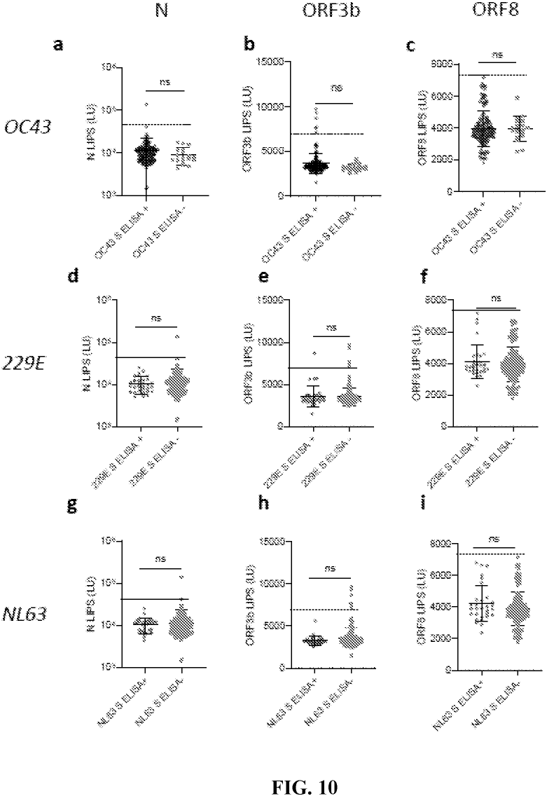

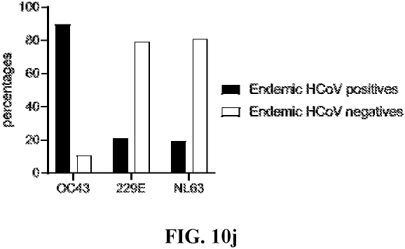

Due to the high prevalence of HCoV, we assessed the positivity for beta-HCoV (OC43) and alpha-HCoV (229E and NL63) Spike antibodies in our negative cohort. We found that 89.6% of our negative cohort is positive for OC43 Spike in ELISA (OD>1.00, FIG. 10J) whilst these samples are true negatives in our SARS-CoV2 LIPS assays with comparable responses to OC43 responders and non-responders (FIGS. 10A-J). The same trend was observed for alpha-HCoV 229E and NL63 with a lower prevalence (21% and 19% Spike IgG positive respectively, FIG. 10J). Similarly, the common beta HCoV HKU-1 was found highly prevalent in the general adult population (98% positive for HKU-1 Spike-Antibodies) in previous studies⁷. When we separate our negative cohort (n=176) into donors with either high or low Spike OC43, 229E or NL63 IgG responses, we find no differences between these groups for N, ORF3b and ORF8 responses by LIPS (FIG. 10). Thus, these results clearly demonstrate the high specificity of our N, ORF3b and ORF8 LIPS tests despite the high prevalence of endemic HCoVs in most samples.

### Discussion

SARS-CoV-2 antibody testing is a major component for diagnosis of recent and past COVID-19 infection. Antibody tests are important for determining infection attack rates in the population, population immunity and informs vaccine development. We report, for the first time, the detection of antibody responses directed against an extensive spectrum of 15 SARS-CoV-2 antigens, to identify new and unique antigenic targets of the humoral immune response of COVID-19 patients using LIPS technology. In our panel, 11 antigens showed elevated antibody responses in COVID-19 patients.

Amongst them, Spike structural protein which is responsible for viral entry and is widely used as a marker of infection. In the cloned Spike subunits S1, S2, S2′, only antibodies to S1 and S2′ were elevated in COVID-19 patients by our LIPS test.

The trimer S conformation and maturation of viral particles by the cleavage of S2 during virus endocytosis to form the S2′ fusion peptide may explain the difference in antigenicity between S2 and S2′.

Furthermore, the assessment of the antibody responses to unique ORFs of SARS-CoV-2 reveal that the combinational use of ORF3b, ORF8 and N is an accurate marker of infection at all time-points. ORF3b and ORF8 are the least identical proteins to SARS-CoV, and homologous proteins do not exist in other strains of HCoV other than sarbecoviruses. However very little is known about their function and expression in SARS-CoV-2. Previous reports found the ORF3b of SARS-CoV plays an important role in the interaction with the innate immune system through inhibition of type 1 interferon synthesis. In SARS-CoV, ORF8 has been shown to accumulate in the endoplasmic reticulum and mediate cell death by autophagy, and associate with the S protein. More importantly, recent findings report that SARS-CoV-2 utilizes ORF8 to alter the expression of MHC-I to evade immune surveillance. A deletion of ORF8 has been reported in a few Singaporean COVID-19 patients, however this lineage has not continued in Singapore and has not been maintained in other countries.

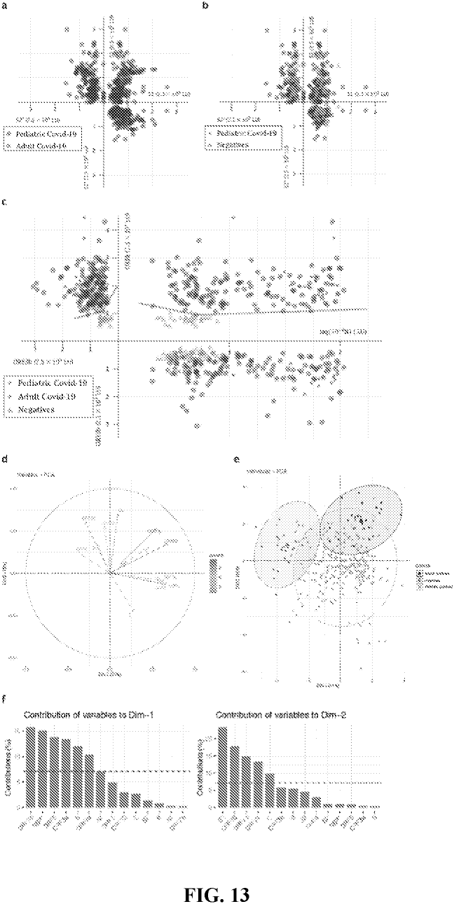

Endemic human coronaviruses are ubiquitous, and sequence homology with other human coronaviruses (HCoV), such as alpha-HCoV strains: 229E and NL63, and beta-HCoV: HKU1 and OC43, could result in the detection of pre-existing cross-reactive antibodies and reduce the specificity of serological assays. However, the structural proteins of SARS-CoV-2, and other common HCoV only share 18 to 40% amino acid homology (table 4). We showed herein that despite the high sero-positivity rate for OC43, 229E and NL63 in our negative cohort, there is an absence of cross-reactivity with our N, ORF8 and ORF3b antibody LIPS tests which is crucial for their wide-use for diagnosis (FIG. 10G and FIG. 13).

Our results need to be confirmed in cohorts from all continents to account for antibody responses across different ethnicities and virus strains. The LIPS platform allowed a broad antibody screening to many antigens, but a translation of the assay into a simpler set-up (e.g. ELISAs) is required for the use in large-scale diagnostics, particularly in resource-poor settings.

In conclusion, our results provide insights into the overall spectrum of antibody responses associated with COVID-19, as patients produce antibodies to structural and non-structural proteins beyond the Spike. There is a need for improvement of current sero-diagnostic tests for detecting infection early after onset of symptoms and for confirmatory assays when existing Spike and N protein Ig ELISAs are found to be positive. We have identified ORF8 antibodies as a major marker of acute, convalescent and long-term antibody response to SARS-CoV-2 infection because of its immunodominance and specificity, in addition to N protein. Moreover, the combined use of ORF3b and ORF8 provides a highly sensitive and specific method for the detection of COVID-19 infections, both early and later in infection. Despite the high prevalence of common cold HCoVs, we have demonstrated the specificity of our assay to SARS-CoV-2. Further investigation on the protective potential of antibodies to these non-structural targets are needed. Such information will help prioritize antigen targets for vaccine development, monoclonal antibody reagents and most importantly detection of early responses to infection by standardized immuno-assays.

### Example 3

### Methods

### Patients and Samples Collection

Our study enrolled a total of 122 children patients and 36 adult patients based on recruitment of available patients with RT-PCR confirmed COVID-19 infection in Hong Kong. We used a total of 254 COVID-19 children plasma samples including 146 longitudinal samples from 58 subjects with 2 to 4 sampling time points, and 119 early time-points samples (< day 14). Samples were used from children (mean±stdev: 39±47 days, range: 0-206 days) and adults (mean±stdev: 54±20 days, range: 24-123 days), with the sample day was defined as day post-symptom onset or RT-PCR confirmation for asymptomatic cases through contact tracing or quarantine. For measurement of IFNα, an extra set of 18 COVID-19 adult patients sampled prior to day 7 was used in comparison to 48 samples that were collected prior to day 7 in the COVID-19 pediatric cohort. The COVID-19 patient study was approved by the institutional review board of the respective hospitals, viz. Kowloon West Cluster (KW/EX-20-039 (144-27)), Kowloon Central / Kowloon East cluster (KC/KE-20-0154/ER2) and HKU/HA Hong Kong West Cluster (UW 20-273, UW20-169), Joint Chinese University of Hong Kong-New Territories East Cluster Clinical Research Ethics Committee (CREC 2020.229). All of patients provided informed consent.

The negative control plasma samples used in this study were from Hong Kong blood donors collected from June to August 2017 (prior to the emergence of COVID-19), used a total of 33 plasma samples including negative pediatric samples (n=20) and negative adult samples (n=13). The collection of negative control blood donors was approved by the Institutional Review Board of The Hong Kong University and the Hong Kong Island West Cluster of Hospitals (approval number: UW16-254). Plasma samples were collected from heparinized blood. All samples from COVID-19 patients or negative controls were heat-inactivated prior to experimental use at 56° C. for 30 minutes. Details on the sample cohort are presented in Table 6.

TABLE 6

Subject cohort Details

| | | | Pediatric COVID-19 | | Adults COVID-19 | | Negative | |
|---|---|---|---|---|---|---|---|---|
| | | | N (%) | | N (%) | | N (%) | |
| Patients | Samples | | 254 | | 36 | | 33 | |
| | Individuals | | 122 | | 36 | | 33 | |
| Symptoms | Asymptomatic | | 44 (36%) | | 9 (25%) | | - | |
| | Mild/Severe | | 78/0 | | 25/1 | | - | |
| | | | Mean±stdev | 10.8±4.9 years | Mean±stdev | 34±17.1 years | Mean±stdev | 29.3±16.3 years |
| Age | | | 0-8 years | 45 (37%) | Median | 31 | Adults (N=13) | 30 - 65 |
| | | | 8-12 years | 33 (27%) | Min | 18 | Pediatrics (N=20) | 11-18 |
| | | | 12-18 years | 44 (36%) | Max | 71 | | |
| Gender | | Female | 49 (40%) | | 10 (28%) | | 17 (51%) | |
| | | Male | 73 (60%) | | 26 (72%) | | 16 (49%) | |

### SARS-CoV-2 Cloning and (Ruc)-Antigen Expression

Based on previous studies describing the structure of the SARS-CoV-2 genome^{25,36}, an extensive panel of 14 proteins (S1, S2, S2′, E, M, N, NSP1, ORF3a, 3b, 6, 7a, 7b, 8, 10) was chosen for antibody testing by LIPS. Primers and cloning for the amplification of SARS-CoV-2 proteins were as previously described Constructs with pREN2-Renilla luciferase plasmid containing the SARS-CoV-2 antigen of interest were transfected into Cos1 cells and prepared as previously described¹⁴.

### Measurement of Antibody Responses Using LIPS

The LIPS assays were performed following the protocol of Burbelo et al., with the following modifications³⁶, as previously described¹⁴. Briefly, (Ruc)-antigen (at an equal concentration for each antigen at 10^7 per well) and plasma (heat inactivated and diluted 1:100) were incubated for 2 hours with shaking at 800 rpm. Ultralink protein A/G beads (Thermo-Fisher) were added to the (Ruc)-antigen and serum mixture in a 96-deep-well polypropylene microtiter plate and incubated for 2 hours with shaking at 800 rpm. The entire volume was then transferred into HTS plates (Millipore) and washed as previously described. The plate was read using QUANTI-Luc Gold substrate (Invivogen) as per manufacturer’s instructions on a Wallac MicroBeta JET luminometer 1450 LSC & Luminescence counter and its software for analysis (PerkinElmer). Experimental controls include no plasma blank wells with (Ruc)-antigens and negative control serum from healthy donors plasma collected prior to the COVID-19 pandemic. The background corresponds to the LU signal from each Ruc-fusion antigen with protein A/G and substrate with no plasma.

### Enzyme-Linked Immunosorbent Assay

Total IgG were measured in plasma samples using the Total human IgG ELISA kit (Thermo-Fisher) at a final dilution of 1:500,000 according to manufacturer’s instructions. IFN-α was measured in plasma samples using the Human IFN-α Platinum ELISA kit (Invitrogen) at a dilution of 1:5 according to manufacturer’s instructions.

### Clusters of Points

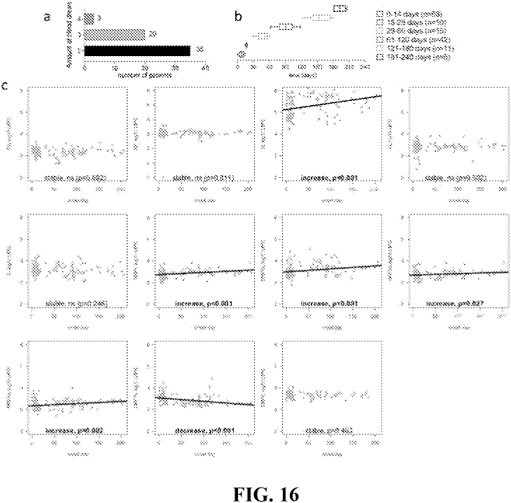

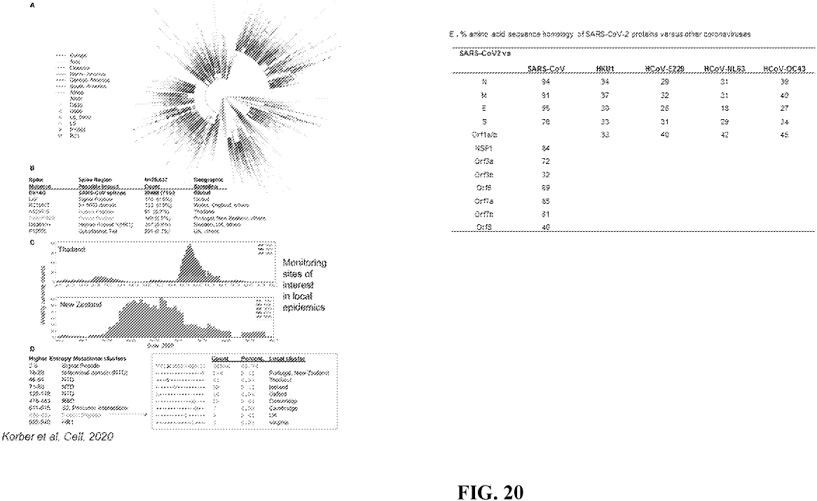

The SARS-CoV-2 antibodies dataset has been treated through the free software ConTeXt, with LuaMetaTeXengine (version 2020.05.18) developed by Hans Hagen (http://www.pragma-ade.nl) which uses TeX, Metapost and Lua to obtain the 3D clusters of points shown in FIG. 16abc, FIG. 20c. For clarity, only the first 144 COVID-19 pediatric samples of the dataset are represented in the clusters of points, along with n=36 COVID-19 adult samples and n=28 negatives. In the cluster (N, ORF3b, ORF8), the equations of the red lines are: (1) in the plane (N, ORF8) :830*log (N) +0.3843*ORF8=4801 and -350*log (N) +1.036*ORF8=790, and (2) in the plane (ORF3b, ORF8): 0.035*ORF3b+0.1334*ORF8=409.284 and 0.074*ORF3b+0.0437*ORF8=221.812. These straight lines allow the most accurate discrimination between negative controls and positive adult populations.

### Principal Component Analysis

The LU for 14 antigens were log-scale transformed (the negative and zero values in the data set were replaced by 1) prior to PCA analysis. The missing values in the dataset were estimated by a probabilistic model ³⁷. The probabilistic model is tolerant to amounts of missing values between 10% to 15% which is fit for our data. The missing data was estimated using pcaMethods (version 1.80.0)³⁸. The completed data were standardized (scaled) before input in standard PCA (using FactoMineR (version 2.4)³⁹.. The PCA results were extracted and visualized using factoextra (version 1.0.7)⁴⁰.

### Statistics and Reproducibility

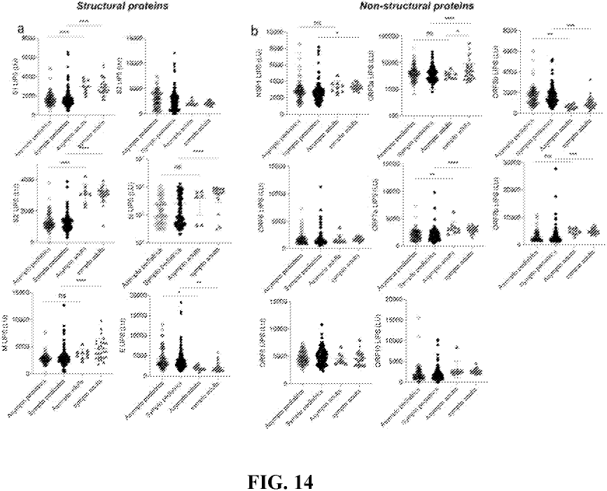

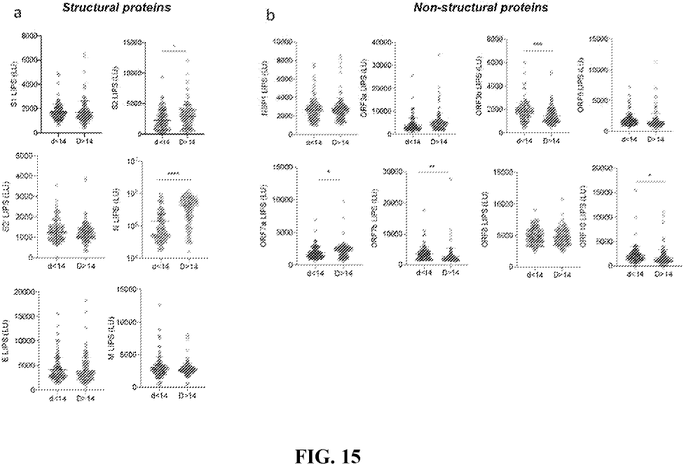

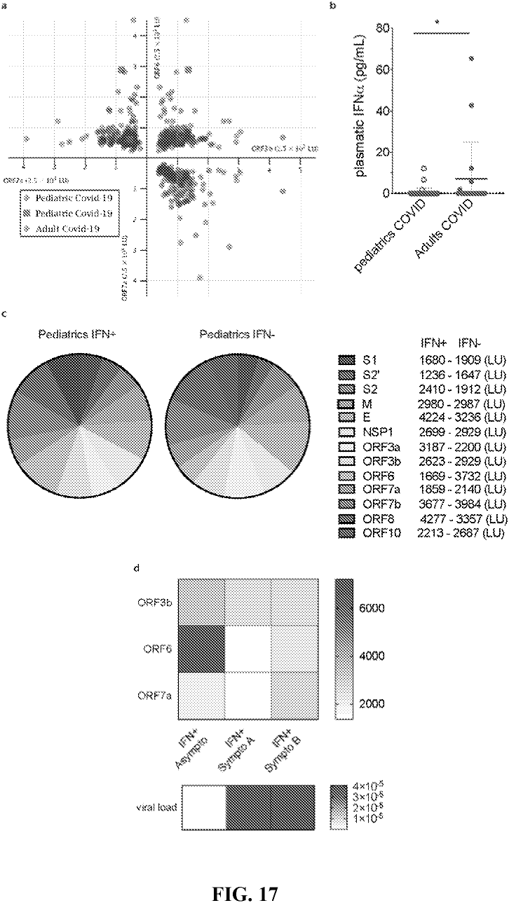

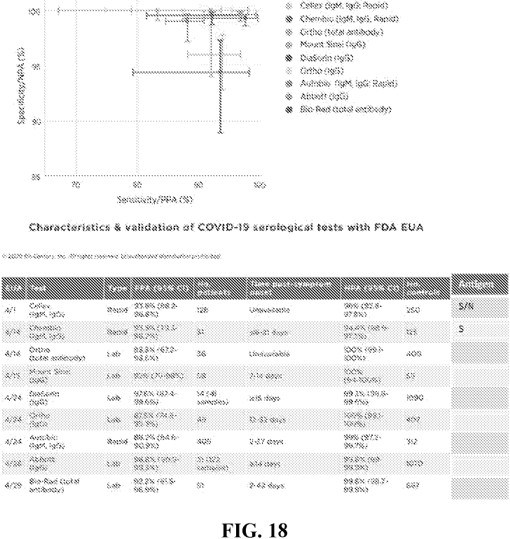

GraphPad Prism version 8 software (San Diego, CA) was used for statistical analysis. All experiments were repeated twice independently. Antibody levels are presented as the individual responses and geometric mean +/- standard deviation (stdev). Ordinary one-way ANOVA with Tukey’s multiple comparison test were performed to compare the pediatric, adult and negative populations in FIGS. 14 and 15, and the early and late samples in FIG. 18. For FIG. 17j, percentages were calculated by dividing each mean antibody value by the sum of the total antibody responses, and compared using a Chi-square test between the “observed” (pediatric) versus “expected” (adult) distributions.

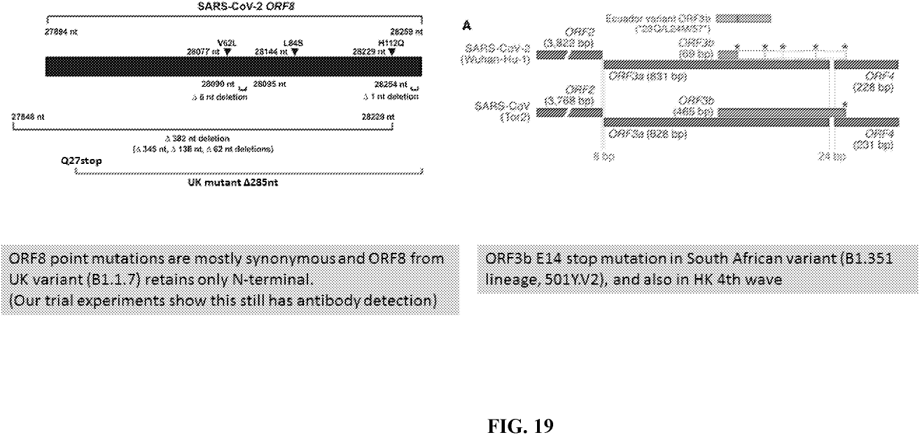

For FIG. 19c and Supplementary FIG. 16, a linear mixed effects model was fitted to account for correlated responses for the longitudinal samples dataset. Log₁₀ LIPS was used for the analysis (as dependent variable) to reduce the impact of extreme values/non-normality.

### Ethics Declaration

The COVID-19 patient study was approved by the institutional review board of the respective hospitals, viz. Kowloon West Cluster (KW/EX-20-039 (144-27)), Kowloon Central / Kowloon East cluster (KC/KE-20-0154/ER2) and HKU/HA Hong Kong West Cluster (UW 20-273, UW20-169), Joint Chinese University of Hong Kong-New Territories East Cluster Clinical Research Ethics Committee (CREC 2020.229). All of patients provided informed consent. The collection of plasma from blood donors serving as controls was approved by Institutional Review Board of The Hong Kong University and the Hong Kong Island West Cluster (UW16-254).

### Results

The SARS-CoV-2 virus emerged in December 2019 and given the lack of pre-existing immunity has caused a pandemic. The spectrum of COVID-19 disease ranges from asymptomatic to lethal infection. It is now evident that the immune response plays a major role in the pathogenicity and outcome COVID-19¹. Children are minimally affected clinically by SARS-CoV-2 and the morbidity and mortality observed in adults increases progressively with age, although the viral loads in the respiratory tract are reportedly comparable between children of all ages and adults². The multisystem inflammatory syndrome (MIS-C) that appears in children after infection with SARS-CoV-2 is a rare exception (0.002% of cases) to the generally milder clinical disease observed³. Symptoms such as fever, cough, pneumonia and elevated C-reactive protein which are associated with disease severity, are less common in children⁴. The majority of children are asymptomatic and only a minority develop mild symptoms (most commonly fever, cough, pharyngitis, gastrointestinal symptoms and anosmia)⁴, creating difficulties in identifying pediatric cases and in contact tracing. These observations are in contrast with other respiratory virus infections (respiratory syncytial virus (RSV), influenza virus) where children are affected more commonly and more severely compared to adults⁵. Recently, a small family case study by Tosif et al., indicated that children can mount an immune response with detectable antibodies to SARS-CoV-2 without preceding detectable viral load, therefore avoiding the development of a symptomatic SARS-CoV-2 infection⁶. The clinical differences observed in children and adults upon SARS-CoV-2 infection may be explained by several immune factors (amongst other clinical or physiological factors), such as pre-existing and cross-reactive immunity to common cold coronaviruses (CCC)⁷ with more recent exposure likely in childdren, immuno-senescence and inflammatory state⁸, innate immune responses, presence of auto-antibodies⁹, and “trained immunity” as a result of off-target effects of live attenuated vaccines for other infections^{5,10}.

Although a growing number of SARS-CoV-2 serology tests are currently in use worldwide and are the basis of the SARS-CoV-2 infection rate data, there is an absence of information on serological responses in children with RT-PCR confirmed SARS-CoV-2 infection. Large epidemiological studies report that children only represent 1-2% of all SARS-CoV-2 cases^{11,12} but this may be and underestimate because of differences in the development of the antibody responses to SARS-CoV-2 in children and pandemic response measures such as school closures. Serology is crucial for determining infection attack rates in the population and for assessing the response to a future vaccine to curb the global pandemic. Most serological tests available rely either on neutralizing antibodies or on the detection of antibodies targeting the Spike (S) or the Nucleocapsid (N) proteins of the virus¹³. We have previously demonstrated that antibodies that are directed against non-structural proteins of the virus, namely ORF3b and ORF8, can be used for accurate diagnosis of SARS-CoV-2 infection¹⁴. Whilst the cellular immune profile of children appears comparable to adults in a small case study⁶, there are no reports on the humoral antibody landscape and kinetics in pediatric cases. There is a lack of information on the SARS-CoV-2 antibody responses in adults or children to the virus accessory proteins, for instance ORF3b, ORF6 and ORF7a, which have been reported to be potent interferon antagonists that may play a role in immune evasion¹⁵⁻¹⁷. A finely tuned and balanced antibody response may impact COVID-19 outcomes, and the breadth and magnitude of the landscapes of antibody responses to non-structural proteins may indicate the extent of virus replication and thus immune control.

In the present study, children and adults with SARS-CoV-2 RT-PCR confirmed infection were used to study the antibody landscape to a comprehensive panel of 14 different structural and accessory proteins by LIPS. We tested a population of 122 infected children and 36 infected adults in Hong Kong, including 58 patients with longitudinal samples (2 to 4 time points, with a range 0 to 206 days post infection) to determine the longevity of the antibody responses. Furthermore, due to intensive contact tracing and case-finding measures in Hong Kong, asymptomatic cases with RT-PCR confirmed infections have been identified and their antibody responses are also profiled. These samples were collected between April to November 2020 and include predominantly the third wave of infection cases in Hong Kong corresponding to the period of July to September 2020. Our data reports to date, the most extensive data on the landscape and kinetics of antibody responses in COVID-19 children.

### SARS-CoV-2 Infected Children Have Lower Levels of Antibodies Than Adults to All Structural Proteins, Except E

We used the unbiased and quantitative LIPS platform to determine the antibody titres to an extensive panel of 14 antigens from structural and non-structural SARS-CoV-2 proteins in plasma samples from a cohort of infected children, in comparison to adults and controls in Hong Kong (Table 6).

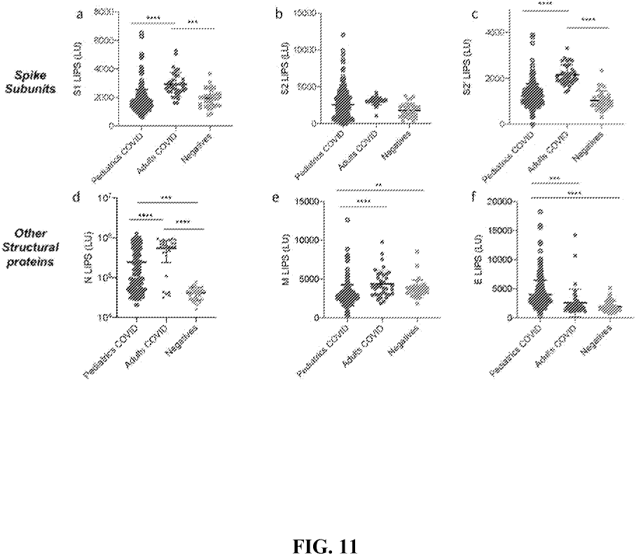

Our first data set represents COVID-19 cases of mixed timepoints and symptoms to determine the overall antibody landscapes in children (mean+/-stdev: 39±47 days, range: 0-206 days), adults (mean±stdev: 54±20 days, range: 24-123 days) and negative controls. S and N antibodies are the most widely used antibodies in COVID-19 serology testing worldwide. We therefore first determined the levels of antibodies to different S sub-units by using 3 different S constructs in the LIPS assay: S1 which contains the RBD domain, S2 and the S2′ cleaved subunit (FIG. 11). The levels of the two Spike antibodies, S1 and S2′ were markedly lower in children compared to the adult cohort (p<0.0001, p=0.0015 and p<0.0001 respectively, FIG. 11AC), whereas no difference was observed for S2 antibodies (FIG. 11B). Moreover, N antibodies were significantly elevated in the pediatric COVID-19 cohort relative to negative controls (2.45e5±2.8e5 LU versus 4.15e4±1.5e4 LU (p=0.0045), but did not reach the levels observed in the COVID-19 adult cohort that were almost half a log above (5.45e5±3.0e5 LU adult COVID-19 cohorts, p<0.0001, FIG. 11D).

We also assessed by LIPS antibodies to other structural proteins Matrix (M) and Envelope (E), which are not widely measured in serology. As for S1, S2′, and N, we found that M antibody levels were lower in the COVID-19 children compared to the adult COVID cohort (p<0.0001, FIG. 11E) but were significantly higher that seen in controls. E antibodies followed an inversed trend as they were significantly elevated in the pediatric COVID-19 cohort (FIG. 11F) compared to both adult COVID-19 (p=0.0006) and negative controls (p<0.0001).

### Increased Breadth of Accessory Antigen Targets in the Pediatric COVID-19 Population

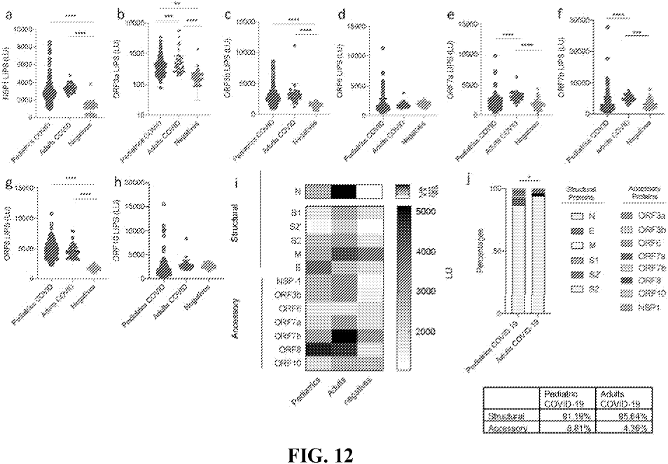

We next investigated the levels of antibodies directed against the non-structural protein 1 (NSP1) and all the ORFs proteins of the virus. In line with our previous study¹⁴, adults with COVID-19 displayed elevated levels of NSP1, ORF3a, ORF3b, ORF7a, ORF7b, and ORF8 antibodies compared to negative controls (p<0.0001, p<0.0001, p<0.0001, p=0.05, p=0.0009, p<0.0001, FIGS. 12a-c and e-g). Again, no detectable levels of ORF6 and ORF 10 antibodies were detected in the adult COVID-19 population (p=0.8691 and p=0.999 respectively, FIGS. 12d and 12h). We observed that the COVID-19 children cohort displayed significantly lower levels of ORF3a, ORF7a, ORF7b antibodies than the adult COVID-19 cohort (p=0.0001, p<0.0001 and p<0.0001 respectively, FIG. 12b, e-f). The magnitude of antibody responses to NSP1, ORF3b and ORF8 were comparable in the pediatric COVID-19 and adult COVID-19 populations, and significantly elevated compared to negative controls (FIG. 12c and g). Cumulative SARS-CoV-2 antibody responses from COVID-19 children and adults populations were then compared as percentages of the total SARS-CoV-2 structural and accessory antibody response. The anti-N antibodies substantially dominate the SARS-CoV-2 humoral response detected by LIPS in both populations (FIGS. 12I-J), which is consistent with our previous findings in the adult population¹⁴. Due to the immunodominant effect of anti-N antibodies, we also performed analysis with or without N, both of which were highly significant (p<0.0001 12J). Furthermore, representation of cumulative percentages of single specific antibodies to the global SARS-CoV-2 antibody response shows that the amount of the response towards the accessory proteins (NSP1 and ORFs) is increased over the response towards the structural ones in the pediatric COVID-19 population versus the adult COVID-19 population (8.81% versus 4.36% for the response to accessory proteins, p=0.019, FIG. 12J) despite no significant differences in total IgG levels in both populations.

### Deciphering the SARS-CoV-2 Antibody Landscape Differences in Children and Adults Using Clusters of Points and Principal Component Analysis

A cluster of points depicts each individual sample in a more complete way than a single statistical comparison, as it considers a combination of three (or more) different parameters taken together and the relevant relations of these parameters. To decipher the SARS-CoV-2 antibody landscape in children, we used relevant antibody combinations to represent the COVID-19 pediatric samples in clusters of points along with the negative and COVID-19 adult populations (FIGS. 13a-c).

First, the cluster representing the three antibodies to the S subunit antigens S1, S2′, S2 confirmed that the pediatric population has a S antibody profile that is more closely comparable to negative controls (FIG. 13A) than an adult COVID-19 response by LIPS (FIG. 13B). Further cluster analysis of antibodies to S1, S2′ with N, or other structural proteins N, M, and E reveals that the COVID-19 children population appears to be quite heterogeneous (Data not shown). Despite having a different profile than both the adult COVID-19 and the negative populations, the pediatric population cannot be clearly discriminated. We then selected accessory protein antibodies as combinations to investigate the relevance of under-utilized markers. ORF3b and ORF8 antibodies were selected, along with N antibodies, as both were previously shown to discriminate accurately COVID-19 adults from negative controls¹⁴. The (N, ORF3b, ORF8) cluster of points can accurately allow the positive discrimination of the pediatric COVID-19 cases from the negatives (FIG. 13C). Indeed, in the (N, ORF8; x, y) plane, the negative population is separated from the adult and pediatric positive ones by two-segments of straight lines (equations of 830*log (N) 0.3843*ORF8=4801 and -350*log (N) +1.036*ORF8=790, with all positive samples represented above or on these lines, and only one negative sample being above these lines). Then, using the (ORF3b, ORF8; y, z) plane, again, two-segments of straight lines (equations of 0.035*ORF3n+0.1334*ORF8=409.284 and 0.074*ORF3b+0.0437*ORF8=221.812) separate the negative samples from the adult and pediatric positive ones. Therefore, the (N, ORF3b, ORF8) cluster reveals that the pediatric COVID population resembles a COVID-19 adult population and can be discriminated from negative pre-pandemic controls. Importantly, this is the only combination that allowed us this discrimination, as other parameter combinations (e.g. (N, S1, S2′), (N, E, M) data not shown) and combinations of antibodies to accessory proteins) were also tested and represented as clusters of points but did not discriminate pediatric samples. These data cluster analysis show that the antibody landscape of the COVID-19 children population is distinct from the adult one.

To test the hypothesis that the antibody landscape to structural and accessory viral proteins drives the distinct profile of the pediatric population, we undertook a principal-component analysis (PCA) of the 14 SARS-CoV-2 antibodies for the full data set (from FIGS. 11 and 13). Dimension (principal component) 1 and 2 explained respectively 21% and 15% of the total variances from all the 14 antibody types (FIGS. 13d-e). Accessory proteins ORF3b, NSP1, ORF8, ORF3a, ORF7a, ORF6 and ORF 10 had high correlation values (Data not shown), reflecting that antibodies to structural proteins do not solely drive the principal component 1. Particularly, contributions of ORF3b, NSP1, ORF8, ORF3a were the highest in Dimension 1 (Dim 1, FIG. 13DE). Moreover, PCA showed that ORF3b and ORF7a antibodies highly contributed to the differences seen in both dimensions (FIG. 13D) highlighting their importance in the serological response.

Strikingly, the PCA revealed that pediatric COVID-19 antibody response was also intermediate between COVID-19 adults and negatives (FIG. 13f). Indeed, the normal-probability representation of the 3 populations showed that only 18.9% of the pediatric patients overlapped with the ellipse of the COVID-19 adults and only 3.54% overlapped with the ellipse of the negatives (FIG. 13f). Further analysis on gender, time-point, symptoms and neutralization data (PRNT90) values reported that these were not significant factors in discriminating the data (Data not shown). Therefore, the differences in the observed SARS-CoV-2 antibody responses is primarily explained by the age and type of patients: pediatric COVID-19, adult COVID-19 or pre-pandemic negative controls.

### No Difference in Antibody Responses Between Symptomatic and Asymptomatic COVID-19

To assess the potential effect of antibodies to structural and non-structural proteins of SARS-CoV-2, we further stratified data (from FIGS. 11 and 12) into symptomatic (mild/severe) and asymptomatic for both the adult and pediatric cohorts.

We found no differences in antibody responses between asymptomatic and mild COVID-19 children for all 14 antigens. In adults, we observed the same trend excluding ORF3a antibody levels which are higher for symptomatic patients (p=0.0403, FIG. 14B). More importantly N, M, ORF3a and ORF7b antibody levels in asymptomatic children versus asymptomatic adults were not significantly different (p=0.5673, p=0.2669, p=0.9185 and p=0.0859 respectively, FIG. 14), whilst symptomatic adults had an upregulated antibody response to these antigens compared to symptomatic children (p<0.0001 for all 4 antigens, FIG. 14).

### Antibody Landscapes at Early Infection and Long-Term Stability

We previously observed that the SARS-CoV-2 antibody responses can vary in magnitude and specificity in adults between acute and convalescent to memory time-points¹⁴. To study the effect of time in the pediatric COVID-19 population, we stratified pediatric responses of all 254 samples (FIGS. 11 and 12) by early (<d14) versus later (≥d14) time-points (FIG. 15). S2, N and ORF7a specific antibodies were significantly increased after day 14 post symptom onset. In contrast, ORF3b and ORF7b antibodies elicited a higher antibody response prior to day 14 (FIG. 15B). Finally, responses to structural proteins S1, S2, M and E and accessory proteins NSP1, ORF3a, ORF6 and ORF8 were comparable before and after day 14 (FIG. 15AB).

To further confirm the stability of SARS-CoV-2 specific antibodies we used 146 longitudinal paired samples of 58 pediatric patients that had either 2, 3 or 4 blood draws (FIG. 16a). The time-frame of sampling ranged from 0 to 206 days post-symptom onset, with the majority of samples from <14 days (n=63), or long term memory samples after day 60 (n=58) (FIG. 16b). Using a linear mixed effects model, we determined that antibody responses to structural proteins S1, S2, S2′, M and E were stable over time, whereas N was significantly increased (p<0.001) (FIG. 16c). Furthermore, antibodies towards non-structural proteins NSP1, ORF3a, ORF3b and ORF7a also significantly increased over time (p<0.001, p=0.001, p=0.027 and p=0.002 respectively), whilst ORF6, ORF8 and ORF 10 were stable (FIG. 16c). Only ORF7b antibody response significantly decayed longitudinally at a slow rate (p<0.001, FIG. 16c). In order to determine whether the slope of each serological marker could inform the disease outcome we compared asymptomatic and symptomatic patients but no significant differences were found (p>0.05 for all) (Data not shown).

### A Distinct Antibody Landscape May Impact IFNα Levels

Severe COVID-19 disease is associated with low IFNα responses in adults which has been linked to the type-I IFN down-regulation roles of ORF3b, ORF7a and ORF6^{15,17}. Antibodies to these 3 proteins in a cluster of points (ORF3b, ORF7a, ORF6 as x,y,z), show that children have a heterogenous humoral profile towards these 3 type-I IFN down-regulators (FIG. 17a). To assess the IFNα response a quantitative ELISA was conducted on plasmas collected before day 7 in children (n=48) and adults (n=18) (FIG. 17b). We observed a significant decrease of acute IFNα levels in children compared to the adult samples (p=0.0165, FIG. 17b), with only 3 pediatric samples showing a detectable IFNα level. To further investigate the antibody profiles with IFNα responses, the total antibody response of these 3 IFNα producing children compared to the non-responder children (n=45), we plotted the average antibody responses to all 14 antigens for each group (Pediatric IFNα⁻ versus Pediatric IFNa⁺, average: FIG. 17c,and individuals: Data not shown). We found a significant difference in the antibody distribution in the 3 IFNα producing children versus the IFNα non-producing children (IFNa⁺ versus IFNa⁻, p<0.0001), with IFNa⁺ children having increased ORF6, ORF7a and ORF3b antibodies, and lower ORF8 and E antibody levels. Notably ORF6 antibody levels were particularly higher in one IFNa⁺ asymptomatic child with low viral loads (FIG. 17d).

### Discussion

Young children account for only a small percentage of reported and medically attended COVID-19 infections⁵, which is unlikely to be completely explained by reduced exposures and school closures. This difference is likely contributed to by differences in host responses between children and adults. We present herein the most comprehensive study to date of the magnitude, specificity and duration of SARS-CoV-2 specific antibodies in children.

While the understanding of immunity to COVID-19 is growing at a fast pace, information on the pediatric population remains limited largely due to their asymptomatic and mild illness compared to the adult population. Furthering our understanding of the immune mechanisms that lead to this mild clinical presentation could represent new alternative therapeutics, prevention methods or improved diagnostics. Our work enables unique serological insights on the long-term response and asymptomatic infections as Hong Kong’s pandemic control strategy has intensive testing, contact tracing and isolation of all COVID-19 cases including asymptomatic and mild cases with longitudinal follow up. This has provided us the opportunity to investigate a cohort of children with both symptomatic and asymptomatic children. We describe the antibody diversity between day 0 and day 206 post-symptom onset, and reveal major differences in the antigens targeted by the humoral immune response of COVID-19 children compared to adults which may indicate differences in viral protein propagation kinetics, pathogenesis and IFN immune evasion. Importantly, we report that the proportion of the antibody response targeting the accessory proteins is significantly increased in children versus adults. Antibodies to the N structural protein largely dominate the humoral immune response both in children and adults, though the total magnitude of the N-specific antibodies in children is substantially lower than adults. Visualization of N antibody levels in our cluster representations clearly show that only half of the pediatric cases have comparable levels of these antibodies to adults. Others have also reported a reduced magnitude of N antibodies in children and they suggested that this observation could be related to a lower release of N proteins related to lower replication in children¹⁸. On the contrary, our data show that children produce antibodies to some accessory proteins (namely NSP1, ORF3b and ORF8) at similar levels to adults, and to structural protein E in higher proportions than adults, the latter notorious for high turnover due to its pivotal role in viral propagation (reviewed in¹⁹). Therefore, these accessory proteins are not being released to a lesser extent in children but may reflect different virus pathogenesis in children compared to adults. Viral loads have been shown to be comparable in children and adults, which may reflect similar levels of viral replication².

For the Spike subunits antibodies, the (S1, S2′, S2) cluster reveals that the children population resembles a negative pre-pandemic population and not a COVID-19 adult one. A recent study describes a lower anti-S IgG, IgM, IgA in the pediatric population which correlates with our findings ²⁰. One explanation on the clinical difference between children and adults raise that the pre-existing immunity against seasonal human coronaviruses (HCoVs) that cross-reacts with SARS-CoV-2 is higher in children, as they have a higher infection rate of seasonal HCoVs than adults². Individuals exposed and unexposed to SARS-CoV-2 have cross-reactive antibodies against the proteins of SARS-CoV-2 and seasonal HCoVs^{22,23}. Moreover because circulating HCoVs have a higher homology to SARS-CoV-2 structural proteins than non-structural proteins (if they exist)^{24,25}, we would expect a higher cross-reactivity for structural proteins based on pre-existing immunity. SARS-CoV-2 infection back-boosts antibodies against conserved epitopes, including the relatively conserved fusion peptide of the Spike S2 subunit^{22,23}. In our hands, COVID-19 children and adults had comparable levels of S2 antibodies, contrary to S1 and S2′, which shows a possible effect of pre-existing HCoVs immunity for more conserved domains of S such as S2.

Our observations of lower Nucleocapsid and Spike antibodies in COVID-19 children may indicate that there may be lower sensitivity of serological detection for SARS-CoV-2 when using assays based on S and/or N alone, leading to an underestimation of SARS-CoV-2 exposed children. S antibodies have been reported in lower magnitude in the majority of mild adult infections, with higher levels being produced in severe cases²⁶, which is consistent with our data on low S antibody levels in children which were also asymptomatic or mild clinical scores. Low antibody levels and low affinity have been associated with Antibody Dependent Enhancement by facilitation of viral uptake by host cells²⁷, however yet no definitive evidence of ADE for SARS-CoV-2 neither in adults nor in children has been brought forward²⁸, but warrants further investigation.

The plane (ORF3b/ORF8) in the cluster of points (N, ORF3b, ORF8) reveals that children samples have specific combinatory values of these two antibodies that is consistent with adult populations, and that makes them distinguishable from the negatives. Similar to NSP1, we report the proportion of all non-structural antibodies makes a greater contribution of the SARS-CoV-2 response in children than in adults. The Principal Component Analysis of our dataset confirmed further the importance of antibodies to accessory proteins in characterising the pediatric samples. Whether these antibodies to accessory proteins play a role in the virus infectivity or in the pathogenesis of the disease and in the milder outcome of SARS-CoV-2 infection in children presents further questions for investigations.

ORF3b, ORF7a and ORF6 proteins have been previously reported to play a role in cellular type-1 IFN down-regulation¹⁵⁻¹⁷. The cluster representation of ORF3b, ORF7a and ORF6 antibodies shows a different pattern between adult and children population. Furthermore, the PCA revealed that ORF7a and ORF3b contributed highly to component 1 and 2 (Dim1 and Dim2) which accounted for 21% and 15% of the variances observed respectively, pointing to a potential pivotal role of these antigens. In all COVID-19 infected children or adults tested at early timepoints of infection (< day 7), the majority did not elicit a detectable IFNα response, in line with previous findings³⁰, but overall children IFNa responses were significantly lower than the adult ones. Amongst the IFNα responders and non-responders in children a different antibody landscape suggests possible functions of these markers in counteracting viral IFN down-regulation, with ORF6 antibody responses doubled in IFNa⁺ children. Bastard et al., reported anti-type I interferon auto-antibodies in a subset of severe COVID-19 patients⁹. Moreover, a recent study has linked high levels of auto-immunity with COVID-19 severe cases in adults². As children are less predisposed to auto-immunity than adults³², it is possible this contributes to their milder clinical presentation along with the diversified antibody landscape observed in our study.

We report in children diverse antibody profiles in early versus late samples and the maintenance or increase of all antibodies to structural and accessory proteins, except ORF7b antibodies, for at least 6 months post-infection. Many factors play a role in antibody long-term persistence, such as antigen release, antigen presentation, induction of a germinal centre reaction and a memory B cell pool³³. Additional studies on viral proteins release, their roles and their specific B cells are needed to fully understand the antibody landscape in children.

Our cohort did not include any case of Multi-inflammatory System in Children (MIS-C). Although one study reported that no distinct antibody response was observed between MIS-C and mild or asymptomatic children¹⁸, it only measured S and N antibodies. Therefore it would be of interest to study the whole spectrum of antibodies in this population and particularly those targeting accessory proteins. In our hands, symptomatic children have significant differences in antibody levels versus symptomatic adults only for selected antibodies (N, M, ORF3a and ORF7b) which suggests that these markers could play a role in infection control or infectivity.

It is possible that the interest for antibodies to SARS-CoV-2 internal proteins will grow with the rollout of sub-unit Spike only vaccines, in order to allow the distinction between SARS-CoV-2 past exposure and vaccination in specific populations and to create an estimated date of exposure given the unique rate of waning of different specificities. The emergence of certain viral mutants such as the ORF8 truncations³⁴ or recent ORF3b deletions³⁵ could modify the contributions of certain ORFs, their antibodies responses could also be used for epidemiology studies on the insurgence of different strains of the virus.

In conclusion, we report the description of a more diversified antibody landscape in the COVID-19 children population compared to adults, with an increased and sustained humoral response to all accessory proteins of the virus. This study of antibody spectrum provides insights into the importance of the breadth of responses and how it differs between children and adult that have diverse outcomes of infection, and could inform improved SARS-CoV-2 diagnostics for the pediatric population.

### References

1. Vabret, N., et al. Immunology of COVID-19: Current State of the Science. Immunity 52, 910-941 (2020).
2. Jones T.C. , M.B., Veith T. , Guido Biele, Marta Zuchowski, Jörg Hoffmann, Angela Stein, Anke Edelmann, Victor Max Corman, Christian Drosten. An analysis of SARS-CoV-2 viral load by patient age. medRxiv (2020).
3. Valverde, I., et al. Acute Cardiovascular Manifestations in 286 Children with Multisystem Inflammatory Syndrome Associated with COVID-19 Infection in Europe. Circulation (2020).
4. Qiu, H., et al. Clinical and epidemiological features of 36 children with coronavirus disease 2019 (COVID-19) in Zhejiang, China: an observational cohort study. Lancet Infect Dis 20, 689-696 (2020).
5. Zimmermann, P. & Curtis, N. Why is COVID-19 less severe in children? A review of the proposed mechanisms underlying the age-related difference in severity of SARS-CoV-2 infections. Arch Dis Child (2020).
6. Tosif, S., et al. Immune responses to SARS-CoV-2 in three children of parents with symptomatic COVID-19. Nat Commun 11, 5703 (2020).
7. Gorse, G.J., Donovan, M.M. & Patel, G.B. Antibodies to coronaviruses are higher in older compared with younger adults and binding antibodies are more sensitive than neutralizing antibodies in identifying coronavirus-associated illnesses. J Med Virol 92, 512-517 (2020).
8. Freeman, T.L. & Swartz, T.H. Targeting the NLRP3 Inflammasome in Severe COVID-19. Front Immunol 11, 1518 (2020).
9. Bastard, P., et al. Autoantibodies against type I IFNs in patients with life-threatening COVID-19. Science 370(2020).
10. Netea, M.G., et al. Trained Immunity: a Tool for Reducing Susceptibility to and the Severity of SARS-CoV-2 Infection. Cell 181, 969-977 (2020).
11. Parri, N., Lenge, M., Buonsenso, D. & Coronavirus Infection in Pediatric Emergency Departments Research, G. Children with Covid-19 in Pediatric Emergency Departments in Italy. N Engl J Med 383, 187-190 (2020).
12. Wu, Z. & McGoogan, J.M. Characteristics of and Important Lessons From the Coronavirus Disease 2019 (COVID-19) Outbreak in China: Summary of a Report of 72314 Cases From the Chinese Center for Disease Control and Prevention. JAMA 323, 1239-1242 (2020).
13. Rikhtegaran Tehrani, Z., et al. Performance of nucleocapsid and spike-based SARS-CoV-2 serologic assays. PLoS One 15, e0237828 (2020).
14. Hachim, A., et al. ORF8 and ORF3b antibodies are accurate serological markers of early and late SARS-CoV-2 infection. Nat Immunol 21, 1293-1301 (2020).
15. Martin-Sancho, L., et al. Functional Landscape of SARS-CoV-2 Cellular Restriction. bioRxiv (2020).
16. Konno, Y, et al. SARS-CoV-2 ORF3b Is a Potent Interferon Antagonist Whose Activity Is Increased by a Naturally Occurring Elongation Variant. Cell Rep 32, 108185 (2020).
17. Yuen, C.K., et al. SARS-CoV-2 nsp13, nsp14, nsp15 and orf6 function as potent interferon antagonists. Emerg Microbes Infect 9, 1418-1428 (2020).
18. Weisberg, S.P., et al. Distinct antibody responses to SARS-CoV-2 in children and adults across the COVID-19 clinical spectrum. Nat Immunol (2020).
19. Schoeman, D. & Fielding, B.C. Is There a Link Between the Pathogenic Human Coronavirus Envelope Protein and Immunopathology? A Review of the Literature. Front Microbiol 11, 2086 (2020).
20. Weisberg, S.P., et al. Distinct antibody responses to SARS-CoV-2 in children and adults across the COVID-19 clinical spectrum. Nat Immunol 22, 25-31 (2021).
21. Monto, A.S., et al. Coronavirus Occurrence and Transmission Over 8 Years in the HIVE Cohort of Households in Michigan. J Infect Dis 222, 9-16 (2020).
22. Shrock, E., et al. Viral epitope profiling of COVID-19 patients reveals cross-reactivity and correlates of severity. Science 370(2020).
23. Ng, K.W., et al. Preexisting and de novo humoral immunity to SARS-CoV-2 in humans. Science (2020).
24. Edridge, A.W.D., et al. Seasonal coronavirus protective immunity is short-lasting. Nat Med 26, 1691-1693 (2020).
25. Chan, J.F., et al. Emerg Microbes Infect 9, 221-236 (2020).
26. Haddad Natalie S , N.D.C., Kuruvilla Merin E, Morrison-Porter Andrea, Fabliha Anam, Kevin S Cashman, Richard P Ramonell, Shuya Kyu, Ankur Singh Saini, Monica Cabrera-Mora, Andrew Derrico, David Alter, John D Roback, Michael Horwath, James B O’Keefe, Henry M Wu, An-Kwok Ian Wong, Alexandra W Dretler, Ria Gripaldo, Andrea N Lane, Hao Wu, Saeyun Lee, Mindy Hernandez, Vanessa Engineer, John Varghese, Sang Le, Iñaki Sanz, John L Daiss, F Eun-Hyung Lee. Elevated SARS-CoV-2 Antibodies Distinguish Severe Disease in Early COVID-19 Infection. bioRxiv (2020).
27. Katzelnick, L.C., et al. Antibody-dependent enhancement of severe dengue disease in humans. Science 358, 929-932 (2017).
28. Guthmiller, J.J., Wilson P. C. Remembering seasonal coronaviruses. Science 370, 1272-1273 (2020).
29. Le Bert, N., et al. SARS-CoV-2-specific T cell immunity in cases of COVID-19 and SARS, and uninfected controls. Nature 584, 457-462 (2020).
30. Galani, I.E., et al. Untuned antiviral immunity in COVID-19 revealed by temporal type I/III interferon patterns and flu comparison. Nat Immunol (2020).
31. Wang, E.Y., Mao, T., Klein, J. , Dai, Y. , Huck, J. D., Liu, F., Zheng, N. S., Ting Zhou, Benjamin Israelow, Patrick Wong, Carolina Lucas, Julio Silva,, et al. Diverse Functional Autoantibodies in Patients with COVID-19. MedRXiv (2020).
32. Glowinska-Olszewska, B., et al. Increasing Co-occurrence of Additional Autoimmune Disorders at Diabetes Type 1 Onset Among Children and Adolescents Diagnosed in Years 2010-2018-Single-Center Study. Front Endocrinol (Lausanne) 11, 476 (2020).
33. Rodda, L.B., et al. Functional SARS-CoV-2-Specific Immune Memory Persists after Mild COVID-19. Cell (2020).
34. Young, B.E., et al. Effects of a major deletion in the SARS-CoV-2 genome on the severity of infection and the inflammatory response: an observational cohort study. Lancet 396, 603-611 (2020).
35. Lam, J.Y., et al. Loss of orf3b in the circulating SARS-CoV-2 strains. Emerg Microbes Infect, 1-678 (2020).
36. Wu, F., et al. A new coronavirus associated with human respiratory disease in China. Nature 579, 265-269 (2020).
37. Burbelo, P.D., Ching, K.H., Klimavicz, C.M. & Iadarola, M.J. Antibody profiling by Luciferase Immunoprecipitation Systems (LIPS). J Vis Exp (JoVE) 32(2009).
38. Roweis, S. EM Algorithms for PCA and Sensible PCA. (1997).
39. Stacklies W., Redestig H., Scholz M., Walther D. & J., S. Methods - a Bioconductor package providing PCA methods for incomplete data. (Bioinformatics, 2007). https://bioconductor.org/packages/release/bioc/html/pcaMethods.html.
40. Lê, S., Josse, J. & Husson, F. FactoMineR: An R Package for Multivariate Analysis. (2008). http://www.jstatsoft.org/v25/i01/
41. R project. https://cran.rproject.org/web/packages/factoextra/index.html

### Example 4

The ORF8 antigen was further tested in a case of SARS-CoV-2 reinfection, as a recombinant protein in ELISA, and for its presence in infected cells.

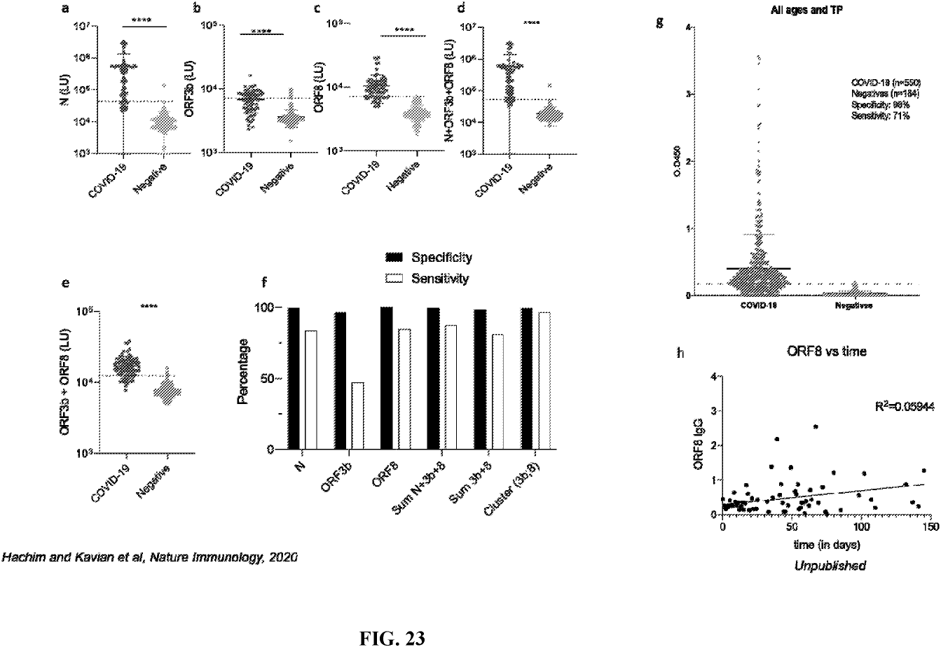

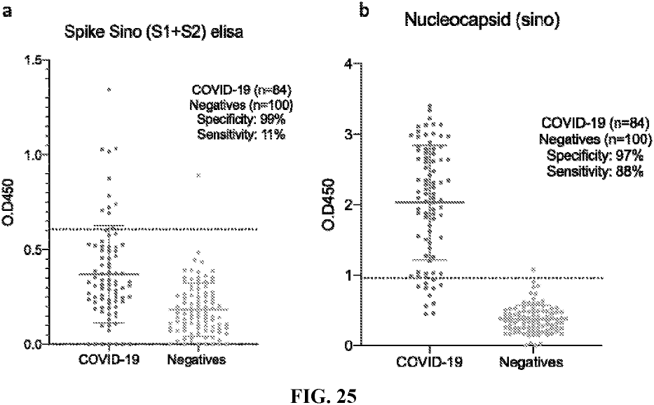

Plasma from a re-infected patient from primary infection (SARS-CoV-2 strain) and secondary infection (ORF8 64E stop) were tested in LIPS for Spike, Nucleocapsid, ORF8 and ORF3b antibodies. The patient initially had a suboptimal antibody response; and antibodies to Spike and Nucleocapsid would not allow accurate diagnosis, whilst ORF8 and ORF8+ORF3b LIPS at d43 were accurate markers of SARS-CoV-2 infection. Moreover, our in-house ORF8 ELISA consolidates this finding (FIG. 23G) with a specificity of 98% and sensitivity of 71% compared to 99% specificity and 11% sensitivity and 97% specificity and 88% sensitivity for Spike (S) and Nucleocapsid (N) in-house ELISA respectively (FIG. 25).

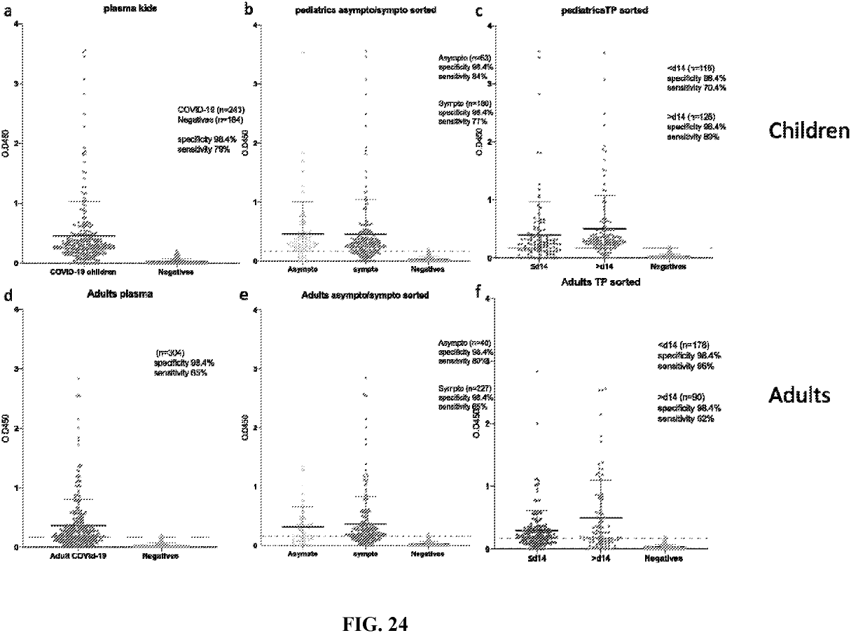

In children, S and N antibody production is suboptimal and can lead to misdiagnosis and an incorrect evaluation of the SARS-CoV-2 infection rates in this population. Our ORF8 ELISA on children plasma reveal a specificity of 98.4% and a sensitivity of 79% highlighting its accurate diagnosis of patients of all ages and at all timepoints (FIGS. 24 A-F).

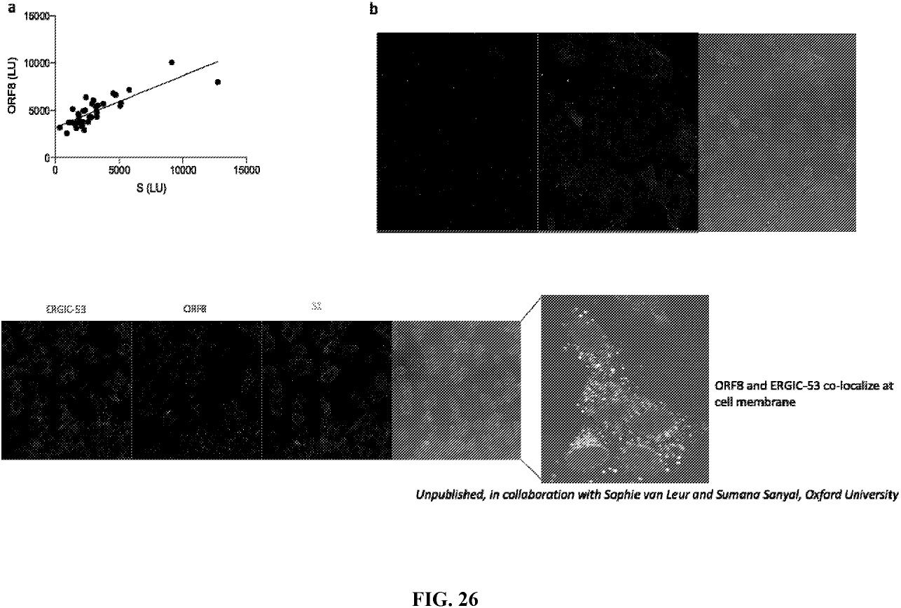

Due to the abundance of ORF8 antibodies and the correlation of Spike and ORF8 antibody titers, we investigated the expression of ORF8 in VeroE6 infected cells. Immunofluorescence staining (IF) shows that ORF8 is abundantly detected in infected cells and co-localizes with Membrane protein and ERGIC-53 (FIGS. 26B and C ). Therefore, our data supports the potential of ORF3b and ORF8 as antigenic test in saliva and/or dried blood for rapid testing.

### Methods

### Patient Samples

Serum from a SARS-CoV-2 reinfection case (Chan EID ref), at 3 timepoints was assessed in LIPS assays for S, N, ORF3b and ORF8 antibody responses.

Plasma samples from Example 2 and 3 were assessed in ELISA assays: 304 Adults COVID-19 plasma and 243 children COVID-19 plasma were assessed against pre-pandemic healthy controls (n=184).

### Enzyme-Linked Immunosorbent Assay

See example 1, with the modification of using tobacco BY-2 cells produced recombinant ORF8 protein (https://doi.org/10.1007/s00299-020-02654-5) from Masashi Mori, used at a coating concentration of 300 ng/mL in coating buffer.

### Measurement of Antibody Responses Using LIPS

See example 1.

### ORF8 Detection in SARS-CoV-2 Infected Cells

Briefly, infected Vero E6 cells cells at MOI 2 24 p.i were stained with enriched ORF8 antibodies for patient plasma, SARS-CoV-2 Spike rabbit monoclonal antibody (Sinobiological), SARS-CoV-2 Membrane mouse monoclonal Antibody (Thermofisher), ERGIC-53 mouse monoclonal antibody (Santa Cruz Biotechnology) as per manufacturer instructions with the following modification: 10% NGS used in all buffers and 3 washes of 20 mn at each step. The slides were then visualised using Carl Zeiss LSM 980.

### Results

Of over 5,000 sequences available on GISAID only limited mutations were found in ORF8 and ORF3b (FIG. 19). It is important to note that the ORF8 deletion mutant that arose in Singapore only infected approximately 10 people before disappearing- showing the importance of ORF8 in virion formation. The ORF8 deletion has not been a stable lineage (FIG. 19)

Common Cold Coronaviruses share high homology with S and N (which can lead to cross-reactivity), whilst ORF8 and ORF3b are specific to SARS-CoV-2 (FIG. 20)

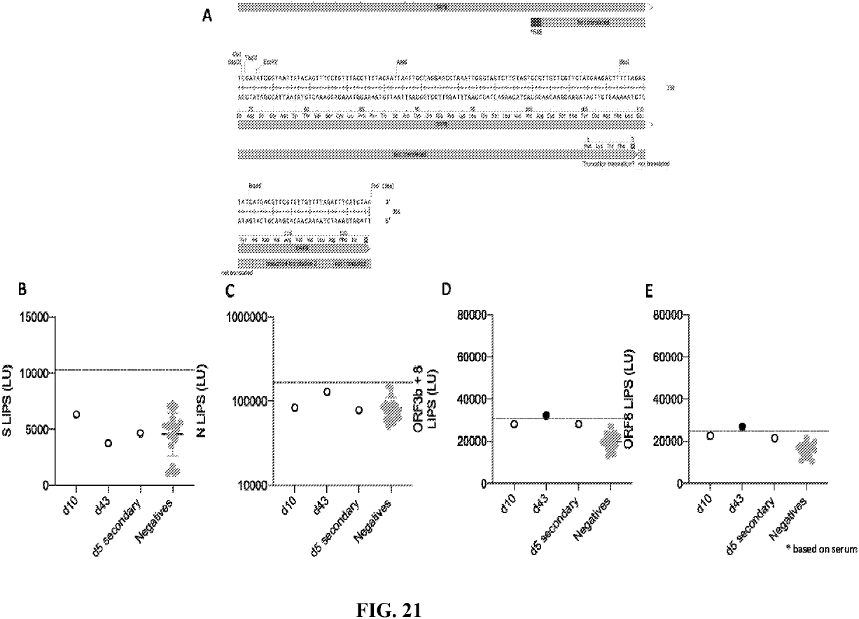

FIG. 21 the study is on a COVID-19 re-infection case in which the 2nd infection has a 64E stop in ORF8 and 1st infection, showing that ORF3b+ORF8 or ORF8 could identify primary infection when S and N antibody tests fail to do so.

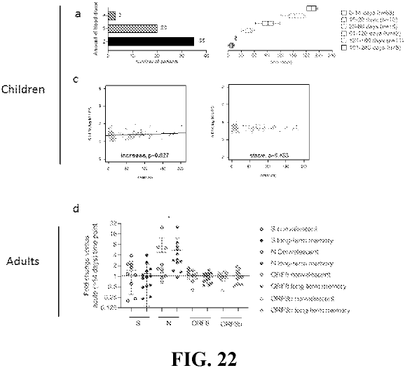

FIG. 22 ORF3b and ORF8 antibody responses are stable overtime in adults (day 0- day 100) and children (day 0- day 204) making them good serological markers at all time-points of infection.

FIG. 23 Both LIPS and ELISA formats confirm that ORF3b+ORF8 testing by LIPS works better than S and N for diagnosis of SARS-CoV-2 infection in children. ORF8 ELISA translation confirms our initial LIPS findings. ORF3b protein in combination ELISA testing is to follow once protein is available.

FIG. 24 Further stratification of data from ORF8 ELISA by age and timepoint of infection: works well in children and > day 14.

FIG. 25 In house ELISA of Spike and Nucleocapsid using SinoBiological proteins for comparison.

FIG. 26 The ORF8 IgG responses are stable with time, by ELISA.

FIG. 27 ORF8 has the potential as an antigen test based on virus localization of infected cells, using enriched patient antibodies for direct detection of ORF8. We also saw that antibody titers for ORF8 and Spike correlated suggesting surface expression of ORF8 of infected cells (Hachim and Kavian et al, Nature Immunology, 2020)

### Conclusion of Examples 1-4

This serology test is based on a groundbreaking discovery of the unique antigens ORF8 and ORF3b in COVID-19 patients and allows to generate diagnostic data distinguishing natural infection from vaccination, which current serology tests based on Ab responses to Spike and Nucleocapsid are not able to work.

The antibody tests described herewith allows:

Detection of infection at both early and late timepoints. ORF8 Abs are detected for a longer period of time than S-antibodies.

These antigens can be used to distinguish infection from vaccination - ORF8 is a non-structural protein and is only present during infection, and ORF3b is unique to SARS-CoV-2 with little homology to other viral ORF3s.

Detection of infection in children with suboptimal antibody responses to S and , therefore ORF3b and ORF8 can be used as further confirmatory testing for borderline cases.

Potential for development as an antigen test to detect infection.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. An immunogenic composition comprising one or more SARS-CoV-2 antigens selected from the group consisting of SARS-CoV-2 proteins or active fragments thereof, which are optionally fused to a light emitting protein.

2. The composition of claim 1, wherein the SARS-CoV-2 antigens are fused to a light emitting protein, optionally, wherein the light-emitting protein is a fluorescent protein or a luciferase.

3. (canceled)

4. The composition of claim 2, wherein the light-emitting protein is a luciferase, wherein the luciferase is selected from the group consisting of Renilla luciferase, a Gaussia luciferase, a modified (optimized) Oplophorus gracilirostris luciferase, a firefly luciferase and a bacterial luciferase.

5. The composition of claim 4, wherein the luciferase is Renilla luciferase.

6. The composition of claim 1, wherein the SARS-CoV-2 proteins are selected from the group consisting of the structural proteins N, M, S, S1, S2′, and NSP1; and the non-structural proteins ORF3a, ORF3b, ORF7a, ORF7b and ORF8.

7. The composition of claim 1, wherein the SARS-CoV-2 proteins are the non-structural proteins ORF8, ORF3b and/or the structural protein N.

8. The composition of claim 1, wherein the SARS-CoV-2 proteins are the non-structural proteins ORF8 and/or ORF3b.

9. (canceled)

10. The composition of claim 1, wherein the SARS-CoV-2 proteins are the non-structural proteins ORF8, ORF3b, NSP1 and ORF7a.

11. An expression vector expressing the SARS-CoV-2 antigens recited in claim 1.

12. The vector of claim 11, wherein the vector comprises at least one nucleic acid sequence of SEQ ID Nos: 2, 3, 5-9, 11-12, 14 and 15.

13. The vector of claim 11, wherein the vector comprises a nucleic acid sequence of SEQ ID No: 3 and/or SEQ ID NO: 6, optionally, wherein the vector comprises: (a) a nucleic acid sequence of SEQ ID No: 3, 6 and/or 7, or (b) SEQ ID No: 3, 6, 9 and/or 14.

14. (canceled)

15. (canceled)

16. A method for detecting an SARS-CoV-2 antigen-specific antibody in a biological fluid sample, comprising:
(i) providing a fusion protein comprising an SARS-CoV-2 antigen fused to a light-emitting protein; wherein the SARS-CoV-2 antigen is selected from the group consisting of SARS-CoV-2 proteins or active fragments thereof;
(ii) contacting the biological fluid sample with the fusion protein, thereby forming an immune complex if the antigen-specific antibody is present in the biological fluid sample;
(iii) contacting the immune complex with beads coated with an immunoglobulin-binding protein to form bead-bound immune complexes; and
(iv) detecting emission of light from the isolated bead-bound immune complexes, thereby detecting the presence of antigen-specific antibody in the biological fluid sample.

17. The method of claim 16, wherein the biological fluid sample is selected from the group consisting of serum, plasma, blood, urine, saliva, and bronchoalveolar lavage fluid.

18. The method of claim 16, wherein the light-emitting protein is a luciferase or a fluorescent protein, optionally, wherein the luciferase is selected from the group consisting of Renilla luciferase, Gaussia luciferase, a modified Oplophorus gracilirostris luciferase, a firefly luciferase, and a bacterial luciferase.

19. (canceled)

20. The method of claim 18, wherein the light-emitting protein is a luciferase, wherein the luciferase is Renilla luciferase.

21. The method of claim 16, wherein the immunoglobulin-binding protein is selected from the group consisting of Protein A, Protein G, Protein A/G, Protein L, and a secondary immunoglobulin molecule, optionally, wherein the immunoglobulin-binding protein comprises a secondary antibody.

22. (canceled)

23. The method of claim 16, wherein the emission of light is detected using a luminometer.

24. The method of claim 16, wherein: (a) the detection of antibody to the SARS-CoV-2 antigens of ORF8 and ORF3b in the biological sample is indicative of a COVID 19 infection and generates diagnostic data distinguishing natural infection from vaccination; (b) detecting SARS-CoV-2 antigen-specific antibody is conducted at an early timepoint prior to day 7 to day 14 since exposure/infection, and/or (c) the biological fluid sample is from a child.

25. (canceled)

26. (canceled)

27. A method for detecting and/or diagnosing and/or treating a subject who has a current exposure to or infection with SARS-CoV-2, comprising the step of contacting a biological fluid sample of the subject with the composition of claim 1, in an assay selected from the group consisting of luciferase Immunoprecipitation system assay, radioimmunoassay, ELISA, immunoprecipitation assay, Western blot, and fluorescent immunoassay.

28. (canceled)

29. A kit for the detection and/or qualitative or quantitative measurement of SARS-CoV-2 antibody in a subject, comprising the composition of claim 1.

30. (canceled)


SEQUENCE LISTING (45 sequences)

SEQ ID NO: 1 - DNA, 29 bp, Artificial Sequence
  Synthetic Construct - Reverse Primer for ORF10
ctgagcggccgcctatgtgagattaaagt

SEQ ID NO: 2 - DNA, 876 bp, SARS-CoV-2
atggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagaaccaatttatgatgaaccgacgacgactactagcgtgcctttgtaa

SEQ ID NO: 3 - DNA, 171 bp, SARS-CoV-2
atggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggcct

SEQ ID NO: 4 - DNA, 228 bp, SARS-CoV-2
atgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaa

SEQ ID NO: 5 - DNA, 668 bp, SARS-CoV-2
atggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagta

SEQ ID NO: 6 - DNA, 366 bp, SARS-CoV-2
atgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgtttaccttttacaattaattgccaggaacctaaattgggtagtcttgtagtgcgttgttcgttctatgaagactttttagagtatcatgacgttcgtgttgttttagatttcatctaa

SEQ ID NO: 7 - DNA, 1260 bp, SARS-CoV-2
atgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaa

SEQ ID NO: 8 - DNA, 3822 bp, SARS-CoV-2
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 9 - DNA, 2013 bp, SARS-CoV-2
atgtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggca

SEQ ID NO: 10 - DNA, 1769 bp, SARS-CoV-2
atgcgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacaca

SEQ ID NO: 11 - DNA, 1378 bp, SARS-CoV-2
atgaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacac

SEQ ID NO: 12 - DNA, 540 bp, SARS-CoV-2
atggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggg

SEQ ID NO: 13 - DNA, 186 bp, SARS-CoV-2
atgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaa

SEQ ID NO: 14 - DNA, 366 bp, SARS-CoV-2
atgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatga

SEQ ID NO: 15 - DNA, 132 bp, SARS-CoV-2
atgattgaactttcattaattgacttctatttgtgctttttagcctttctgctattccttgttttaattatgcttattatcttttggttctcacttgaactgcaagatcataatgaaacttgtcacgcctaa

SEQ ID NO: 16 - DNA, 117 bp, SARS-CoV-2
atgggctatataaacgttttcgcttttccgtttacgatatatagtctactcttgtgcagaatgaattctcgtaactacatagcacaagtagatgtagttaactttaatctcacatag

SEQ ID NO: 17 - DNA, 31 bp, Artificial Sequence
  Synthetic Construct - NSP1 (ORF1a/b) Forward Primer
ctgagcggatccatggagagccttgtccctg

SEQ ID NO: 18 - DNA, 30 bp, Artificial Sequence
  Synthetic Construct - NSP1 (ORF1a/b) Reverse Primer
ctgagcggccgcccctccgttaagctcacg

SEQ ID NO: 19 - DNA, 35 bp, Artificial Sequence
  Synthetic Construct - S Forward Primer
ctgactcgagatgtttgtttttcttgttttattgc

SEQ ID NO: 20 - DNA, 34 bp, Artificial Sequence
  Synthetic Construct - S Reverse Primer
ctgaggtaccttatgtgtaatgtaatttgactcc

SEQ ID NO: 21 - DNA, 25 bp, Artificial Sequence
  Synthetic Construct - S1 Forward Primer
ctgactcgagatgtgtgttaatctt

SEQ ID NO: 22 - DNA, 32 bp, Artificial Sequence
  Synthetic Construct - S1 Reverse Primer
ctgactcgagatgcgtagtgtagctagtcaat

SEQ ID NO: 23 - DNA, 32 bp, Artificial Sequence; identical to SEQ ID NO: 22
  Synthetic Construct - S2 Forward Primer

SEQ ID NO: 24 - DNA, 30 bp, Artificial Sequence
  Synthetic Construct - S2 Reverse Primer
ctgaggtacctgtgtaatgtaatttgactc

SEQ ID NO: 25 - DNA, 30 bp, Artificial Sequence
  Synthetic Construct - S2' Forward Primer
ctgactcgagatgaggtcatttattgaaga

SEQ ID NO: 26 - DNA, 30 bp, Artificial Sequence; identical to SEQ ID NO: 24
  Synthetic Construct - S2' Reverse Primer

SEQ ID NO: 27 - DNA, 29 bp, Artificial Sequence
  Synthetic Construct - NP Forward Primer
ctgagcggatccatgtctgataatggacc

SEQ ID NO: 28 - DNA, 32 bp, Artificial Sequence
  Synthetic Construct - NP Reverse Primer
ctgagcggccgcttaggcctgagttgagtcag

SEQ ID NO: 29 - DNA, 34 bp, Artificial Sequence
  Synthetic Construct - E Forward Primer
ctgagcggatcctgtactcattcgtttcggaaga

SEQ ID NO: 30 - DNA, 32 bp, Artificial Sequence
  Synthetic Construct - E Reverse Primer
ctgagcggccgcgaccagaagatcaggaactc

SEQ ID NO: 31 - DNA, 25 bp, Artificial Sequence
  Synthetic Construct - M Forward Primer
ctgagcggcagattccaacggtact

SEQ ID NO: 32 - DNA, 36 bp, Artificial Sequence
  Synthetic Construct - M Reverse Primer
ctgagcggccgcgcaaagcaatattgtcactgctac

SEQ ID NO: 33 - DNA, 29 bp, Artificial Sequence
  Synthetic Construct - ORF3a Forward Primer
ctgagcggatccatggatttgtttatgag

SEQ ID NO: 34 - DNA, 34 bp, Artificial Sequence
  Synthetic Construct - ORF3a Reverse Primer
ctgagcggccgcttacaaaggcacgctagtagtc

SEQ ID NO: 35 - DNA, 29 bp, Artificial Sequence
  Synthetic Construct - ORF3b Forward Primer
ctgagcggatccatggcttattgttggcg

SEQ ID NO: 36 - DNA, 29 bp, Artificial Sequence
  Synthetic Construct - ORF3b Reverse Primer
ctgagcggccgcaggccagcagcaacgag

SEQ ID NO: 37 - DNA, 28 bp, Artificial Sequence
  Synthetic Construct - ORF6 Forward Primer
ctgagcggatccatgtttcatctcgttg

SEQ ID NO: 38 - DNA, 29 bp, Artificial Sequence
  Synthetic Construct - ORF6 Reverse Primer
ctgagcggccgcttaatcaatctccattg

SEQ ID NO: 39 - DNA, 27 bp, Artificial Sequence
  Synthetic Construct - ORF7a Forward Primer
ctgagcggatccatgaaaattattctt

SEQ ID NO: 40 - DNA, 29 bp, Artificial Sequence
  Synthetic Construct - ORF7a Reverse Primer
ctgagcggccgctcattctgtctttcttt

SEQ ID NO: 41 - DNA, 29 bp, Artificial Sequence
  Synthetic Construct - ORF7b Forward Primer
ctgagcggatccatgattgaactttcatt

SEQ ID NO: 42 - DNA, 30 bp, Artificial Sequence
  Synthetic Construct - ORF7b Reverse Primer
ctgagcggccgcttaggcgtgacaagtttc

SEQ ID NO: 43 - DNA, 30 bp, Artificial Sequence
  Synthetic Construct - ORF8 Forward Primer
ctgagcggatccatgaaatttcttgttttc

SEQ ID NO: 44 - DNA, 30 bp, Artificial Sequence
  Synthetic Construct - ORF8 Reverse Primer
ctgagcggccgcttagatgaaatctaaaac

SEQ ID NO: 45 - DNA, 29 bp, Artificial Sequence
  Synthetic Construct - ORF10 Forward Primer
ctgagcggatccatgggctatataaacgt